(19) United States
(12) Patent Application Publication
(10) Pub. No.: US 2025/0345635 A1
(43) Pub. Date: Nov. 13, 2025

(54) ULTRASOUND ARRAYS FOR ENHANCED TISSUE THERAPY

(71) Applicant: Alpheus Medical, Inc., Chanhassen, MN (US)

(72) Inventors: Vijay Agarwal, New York, NY (US); Jeremy Ling, Mendota Heights, MN (US); Troy M. Coolidge, Victoria, MN (US); Braden J. Eliason, Minneapolis, MN (US); John R. Ballard, Waconia, MN (US); Gregg Miller, Boulder, CO (US)

(21) Appl. No.: 18/868,081

(22) PCT Filed: May 25, 2023
(86) PCT No.: PCT/US2023/023558

§ 371 Date: Nov. 21, 2024

Related U.S. Application Data
(60) Provisional application No. 63/346,192, filed on May 26, 2022.

(51) Int. Cl. A61N 7/00

(52) U.S. Cl. CPC A61N 7/00; A61N 2007/0078

(57) ABSTRACT

Ultrasound transducer arrays are provided to for example, initiate and enhance therapeutic treatments with a normalized, randomized, and/or incoherent acoustic pressure field. Optimizing pressure uniformity (peak and average pressures) throughout the incoherent pressure field, and maximizing volume of that incoherent field may be achieved through controller filtering steps to assign unique element waveform phases for each element in the array. Ultrasound may be used alone, to activate a drug, pro drug, sonosensitizer, and/or microbubble additives, and can be combined with other energy (e.g., radiation, magnetism), for treatments including cancer, neurological disease, mood condition, sleep apnea, inflammation, and/or orthopedic diseases, and opening the blood brain barrier to improve access to the drugs and additives. Ultrasound transducer systems may used with a cooling system, an alignment device a monitoring system an authorization system (e.g., identification bar code, key) and/or a treatment planning system.

### INCORPORATION BY REFERENCE TO ANY PRIORITY APPLICATIONS

This application claims priority from U.S. Provisional Patent Application 63/346,192 filed May 26, 2022 and titled Ultrasound Arrays For Enhanced Tissue Therapy, which is hereby incorporated by reference in its entirety, herein.

### BACKGROUND

### Field of the Invention

This document relates to methods and apparatuses for activating certain compounds to treat cancer and other diseases, wherein activation involves application of energy (such as ultrasound).

### Description of the Related Art

Ultrasound therapy is used to treat tissue. In some instances, ultrasound has been used to activate certain compounds to treat, for example, cancer. For example, Applicant's filings PCT International Application No. PCT/US2020/017983 entitled Non-Invasive Sonodynamic Therapy filed Feb. 12, 2020 and PCT International Application No. PCT/US2021/071101 entitled Ultrasound Arrays For Enhanced Sonodynamic Therapy For Treating Cancer filed Aug. 4, 2021 disclose the use of ultrasound.

### SUMMARY

As discussed above, Applicant's filings PCT International Application No. PCT/US2020/017983 entitled Non-Invasive Sonodynamic Therapy filed Feb. 12, 2020 and PCT International Application No. PCT/US2021/071101 entitled Ultrasound Arrays For Enhanced Sonodynamic Therapy For Treating Cancer filed Aug. 4, 2021 (herein incorporated by reference) disclose the use of ultrasound.

As disclosed in several embodiments herein, ultrasound transducer arrays are configured to generate ensonification drive patterns for initiating and enhancing therapeutic treatments with a normalized, randomized, and/or incoherent acoustic pressure field. Ultrasound can be used for treatment alone, such as for treating cancer, neurological disease, mood condition, sleep apnea, inflammation and orthopedic diseases, and for opening the blood brain barrier. In some embodiments, ultrasound is used to activate a drug, pro drug, sonosensitizer, and/or microbubble additives. Several embodiments provide a system and use thereof of an ultrasound device that delivers ultrasound in a manner that reduces damage to non-target (e.g., healthy) tissue by delivering ultrasound to the targeted tissue (such as tumor tissue) by uniformly and more consistently creating an incoherent treatment region (e.g., volume) by reducing “hot spots” (e.g., pressure spikes). In one embodiment, one or more parameters are controlled to smooth out, temper and/or otherwise reduce the spikes/variability/extremes in pressure. In other words, in some embodiments, the pressure is normalized to reduce both the peaks and valleys, which in turn, moderates temperature. These parameters include for example, modulation of phase and/or frequency to create incoherent, normalized treatment areas. Reducing hot spots in tissue can be especially important in sensitive areas, such as the brain. Defocused or unfocused ultrasound is used (instead of focused ultrasound) in many embodiments.

Optimizing pressure uniformity (peak and average pressures) throughout the incoherent pressure field, and maximizing volume of that incoherent field can be achieved through controller filtering steps to assign preferred and unique element waveform phases for each element in the array. Ultrasound may be used alone, to activate a drug, pro drug, sonosensitizer, and/or microbubble additives, and can be combined with other energy (e.g., radiation, magnetism), for treatments including cancer, neurological disease (e.g., Alzheimer's and others), mood condition, sleep apnea, inflammation, and/or orthopedic diseases, and opening the blood brain barrier to improve access to the drugs and additives. In various embodiments, ultrasound transducer systems are used with a cooling system, an alignment device (e.g., marking system, fiducial marks, magnetic tracking, automated alignment devices, imaging system, etc.) a monitoring system (e.g., temperature monitor, reflection monitor, cavitation monitor, imaging device) an authorization system (e.g., identification bar code, key) and/or a treatment planning system (e.g., imaging scan data via camera, CT, MRI, simulation software).

In various embodiments, ultrasound is used to treat tissue, including for example, tissue in the brain, lung, breast, colorectal region, prostate, bladder, ovary, testicle, pancreas, liver, stomach, intestine, colon, bone, and/or spine may be treated using for example, one or more ultrasound parameters described herein. The treatment target may be cancerous or benign. In several embodiments, the systems and methods described herein are used for both human and veterinary applications, including for example, canine, feline and equine applications.

In some embodiments, ultrasound is combined with other energy (e.g., sonic, light, ultraviolet, infrared, electric, magnetic, electromagnetic, radiofrequency, and other forms of energy). Systems can be used for treatments including cancer, neurological disease (e.g., Alzheimer's and others), mood condition, sleep apnea, inflammation, and/or orthopedic diseases, and opening the blood brain barrier to improve access to the drugs and additives. In various embodiments, ultrasound transducer systems are used with a cooling system, an alignment device (e.g., marking system, fiducial marks, magnetic tracking, automated alignment devices, imaging system, etc.) a monitoring system (e.g., temperature monitor, reflection monitor, cavitation monitor, imaging device), an authorization system (e.g., identification code, bar code, hologram for drug, key, and/or component for authorized operation of the system) and/or a treatment planning system (e.g., imaging scan data via camera, CT, MRI, simulation software).

In some embodiments, ultrasound therapy is used to activate a drug, prodrug, sonosensitizer, and/or microbubble additive that selectively accumulates in cells within the tissue for treatment. In one embodiment, a sonosensitizing agent (e.g., drug, prodrug, sonosensitizer, microbubble additive) preferentially accumulates in the cells of tumors, lesions, damaged or affected tissue. The sonosensitizing agent can increase a quantity, accumulation, or concentration of a sonosensitizer in the tissue. In one embodiment, microbubbles are administered in conjunction with ultrasound to increase cavitation activity and thereby lower the activation energy threshold for sonosensitizer activation. Commercial ultrasound agents, such as contrast agents, currently available for blood-brain-barrier (BBB) opening in humans include lipid-stabilized microbubbles. Microbubble agents can be administered intravenously, via injection, or administered orally. For example, microbubble agents can be manufactured from biocompatible materials and administered in a diluted solution with saline, and the bubbles are circulated through the vasculature to arrive a target therapy location. The microbubbles can be used to enhance ultrasound and/or as a therapeutic agent. Microbubbles can be collapsed via cavitation by exposure to ultrasound, which can be used for targeted compound delivery and enhancing therapies. In various embodiments, when ultrasound is applied to microbubble agents, cavitation activity is increased in the therapeutic field, which results in increased membrane permeability (through various membranes depending on the target region, such as the brain, CNS, and other organs and orifices), localized temperature increases, and/or broader activation of the sonosensitizer. Ultrasound is used at a frequency of 200-2000 kHz, 500-1500 kHz or 600-1200 kHz in some embodiments. Ultrasound is used, in several embodiments, to enhance delivery of agents to target regions by increasing penetration of the agent(s). Agents include one or more of sonosensitizers, microbubble agents, cavitation agents, chemotherapy agents, immunotherapy agents, antibodies, viruses (such as oncolytic viruses) drugs, etc. according to several embodiments. Transient opening and/or increased permeability of the blood brain barrier is one example. Other examples include tumor tissue at other locations (liver, pancreas, breast, GI, reproductive system, etc.). The transient opening of the BBB can occur after ultrasound exposure for 10 seconds to 10 minutes (e.g., 10, 30, 45, 60 seconds, 2, 3, 4, 5, 6, 7, 8, 9, or 10 minutes, 12-48, 18-29, 32-46 seconds, 1-7 minutes, 2-5 minutes, 3-8 minutes, and values and ranges therein) which increase permeability of the BBB for a period of 10 minutes to 48 hours, (e.g., 10, 15, 20, 25, 30, 35, 40, 45, 50, and 60 minutes, 1-5, 3-9, 12-22, 19-28, 38-54, and 42-57, 5-60, 5-120, 10-60, 10-120 minutes, 1, 2, 3, 4, 5, 6, 7, 10, 12, 15, 20, 24, 30, 36, 40, 45, 48 hours, and values and ranges therein). Penetration of the agent according to several embodiments may be increased by 25-1000%, 50-800%, 100-600%, 200-400%, 25-100%, 50-75%, 66-88%, and other values and ranges therein as compared to not using ultrasound, and in some embodiments, penetration may be increased 2-10 fold or more. In some embodiments, one or more applications (e.g., 1, 2, 4, 5, 6, 8, 10 applications) of the ultrasound to open the BBB. In various embodiments, the opening of the blood brain barrier allows access to certain agents and not others and is thus a selective opening of the blood brain barrier, wherein said selectivity is based on one or more of the following: type of agent, size of agent, molecular weight of agent, transporter associated with agent, or polarity of agent.

In some embodiments, ultrasound therapy is used in conjunction with an auxiliary or additional energy (e.g., sonic, light, ultraviolet, infrared, electric, magnetic, electromagnetic, radiofrequency, and other forms of energy).

Several embodiments described herein are used synergistically with other cancer therapies, including for example, radiation, chemotherapy, immunotherapy, and cell therapies. For example, a combination of ultrasound and a sonosensitizer as described herein can reduce or eliminate the need for one or more additional complementary treatments. For example, lower doses or fewer additional treatments of chemotherapy, radiation, cell therapy, etc. may be needed when cancerous tissue is treated by the combination of ultrasound and a sonosensitizer as described herein, thus enhancing patient care and reducing side effects. As described herein, in several embodiments, tumors are treated using sonosensitizers and ultrasound, wherein the ultrasound activates the sonosensitizer with cavitational and/or thermal energy to produce reactive oxygen species that is cytotoxic to cancer cells and interacts with other molecules to intentionally damage cancer cells via oxidation and associated thermal, chemical, and/or luminescent phenomena for enhancing a cytotoxic effect, stressing and/or inhibiting repair mechanisms of cancer cells, such as by affecting cancer cell production of Heme, removing iron ions, and/or inhibiting the action of ferrochelatase. Advantageously, in one embodiment, a sonodynamic therapy system delivers a signal that is attenuated and/or enhanced to reduce the peak amount of energy needed to destroy cancer cells, therapy limiting damage to surrounding healthy cells. In various embodiments, the sonodynamic therapy system generates electric drive signals to form modulated, incoherent acoustic wave parameters at relatively low energy intensity and frequency. In one embodiment, the ultrasound energy is not focused, thus simplifying the efficient treatment of larger areas of target tissue. In one embodiment, complementary treatment further augments the effectiveness of the sonodynamic cancer treatment. Low intensity, dispersed, non-focused sonodynamic therapy that is delivered through a comfortable, flexible patient interface that conforms to the patient's body allows for targeted treatment of undesired tissue while preserving healthy tissue.

In one embodiment, a target tissue for treatment is treated at a single site. In various embodiments, a target tissue is treated at one or more sites, such as at 1, 2, 3, 4, 5, 10, 15, 20, 25, 30, 50, 100, 200, 500, 1000 or more sites, in ranges of 1-1000, 1-500, 1-100, 1-50, 1-25, 1-10, and 1-5 sites (with any values and ranges therein). In one embodiment, sequential sonodynamic treatments affect a first portion of a target tissue, a second portions of the target tissue, and any subsequent portions of the target tissue. In one embodiment, a target tissue is partially treated or extracted, and then subsequent treatment(s) treat the remaining target tissue at one or more sites. In one embodiment, a target tissue is partially treated or extracted at a core or central portion, and then subsequent treatment(s) treat the remaining target tissue at one or more sites along the periphery of the target tissue. In one embodiment, a portion of a target tissue is treated, with the target tissue treated portion being 1%, 5%, 10%, 15%, 20%, 25%, 30%, 40%, 50%, 60%, 70%, 75%, 80%, 85%, 90%, 95%, 96%, 97%, 98%, 99%, 100%, and any values and ranges therein (e.g., 1-100%, 1-50%, 1-75%, 1-25%, 1-10%, 10-20%, 20-30%, 30-40%, 40-50%, 50-60%, 60-70%, 70-80%, 80-90%, 80-100%, 90-100%, 90-95%, 90-96%, 90-97%, 90-98%, 90-99%, 25-50%, 50-75%, 25-75%, 25-100%, 50-100%, 75-100%, etc.).

In one embodiment a targeting template is placed on the patient to facilitate alignment of the transducer to the various treatment sites. In various embodiments, the targeting template is a wearable elastic template with markers (e.g., fiducial markers, magnetic markers, etc.) to facilitate treatment, such as by demarking a grid, positions based on anatomy, or marking of the skin with indicators. In one embodiment, a surgical ruler is used with marks/markers to assist in the measuring and marking of treatment sites on the patient. In one embodiment, treatment site locations are pre-operatively planned to accomplish full therapeutic coverage of the diseased organ. In one embodiment, the targeting template is a cap. In one embodiment, the targeting template is a band configured to wrap around a head, neck, chest, torso, back, waist, leg, buttock, genital area or other body part. In one embodiment, the targeting template is drawn on the body (e.g., ink, wax, make up, pencil, charcoal, tattoo (e.g., indelible and/or permanent), sticker, tab, or other marking). In one embodiment, the targeting template includes measurement gradients that allow the user to customize treatment locations to patient specific anatomical size. In some embodiments, the targeting template remains in place during ultrasound treatment. In some embodiments, the targeting template is made to be removable prior to ultrasound treatment.

In addition to treating brain cancer, cancerous tissue in the lung, breast, colorectal region, prostate, bladder, stomach, and pancreas may be treated using several embodiments described herein using for example, one or more sonosensitizers along with the ultrasound parameters described herein. Ovarian cancer is treated in some embodiments. Tumors that are difficult to access including those surrounded by bony structures are treated in various embodiments, including but not limited to brain or spinal tumors. Treatment of undesired tissue in joints and other orthopedic applications are also provided herein. In some embodiments, sonodynamic therapy is used to improve efficiency of chemotherapeutic molecules, sonoporation, and/or gene delivery.

In several embodiments, a system for sonodynamic therapy includes at least one ultrasound transducer array housed with a patient interface to acoustically couple the transducer to a patient. A controller coupled to the transducer is configured to generate an electrical drive signal from a set of modulated acoustic wave parameters, calibrate and/or modulate the drive signal for each element in an ultrasonic array, drive the transducer at a frequency to produce a modulated acoustic wave to produce an acoustic intensity sufficient to activate a sonosensitizer in a treatment region, and/or work with a complementary therapeutic system. Several embodiments of ensonification drive patterns using incoherent acoustic fields do not require beam focusing, and thus reduce the need for accuracy and expense with small area focused ultrasound technology and/or high resolution imaging or diagnostics. Thus, in some embodiments, one or more sonosensitizers is administered to a patient without imaging the location of the sonosensitizer(s) or its products, by-products, and/or metabolites (such as for tumor location purposes). Low intensity, dispersed, non-focused sonodynamic therapy that is delivered, according to one embodiment, through a comfortable interface such as a flexible patient interface that conforms to the patient's body allows for lower dosage over more time. A patient interface may include alignment features and anatomical landmarks to simplify cancer treatment in a hospital or office setting.

In various embodiments, sonodynamic therapy with an ultrasound array delivering a temporal-average intensity output below 8, 10, 15, 20 W/cm² (e.g., 0.1-8 W/cm², 0.1-4 W/cm², 0.5-5 W/cm² etc., and values and ranges therein) to cancer tissue can be used to induce and activate sonosensitizer at relative deep depths within a patient's body with or without cavitation and/or thermal effects and/or sonoluminescence to produce reactive oxygen species, intracellular singlet oxygen, and/or free radicals in a cascade of events that activate the sonosensitizer and in turn damage the cancer cells. In various embodiments, sonodynamic therapy with an ultrasound array delivering a pulse-average intensity output below 8, 10, 15, 20 W/cm² (e.g., 0.1-8 W/cm². 0.1-4 W/cm², 0.5-5 W/cm² etc., and values and ranges therein) to cancer tissue can be used to induce and activate sonosensitizer at relative deep depths within a patient's body with or without cavitation and/or thermal effects and/or sonoluminescence to produce reactive oxygen species, intracellular singlet oxygen, and/or free radicals in a cascade of events that activate the sonosensitizer and in turn damage the cancer cells. In various embodiments, sonodynamic therapy can be used with or without other therapies, such as photodynamic therapy. In some embodiments, ultrasound is delivered at a temporal-average intensity output below 8, 10, 15, 20 W/cm² (e.g., 0.1-8 W/cm², 0.1-4 W/cm², 0.5-5 W/cm² etc. and values and ranges therein) to target tissue with chemical, thermal, cavitation and/or sonoluminescence therapy to damage the target tissue (e.g., cancer cells). In some embodiments, ultrasound is delivered at a pulse-average intensity output below 8, 10, 15, 20 W/cm² (e.g., 0.1-8 W/cm², 0.1-4 W/cm², 0.5-5 W/cm² etc. and values and ranges therein) to target tissue with chemical, thermal, cavitation and/or sonoluminescence therapy to damage the target tissue (e.g., cancer cells).

Several embodiments described herein are used synergistically with other cancer therapies, including for example, radiation, chemotherapy, and cell therapy. In one embodiment, the combination of ultrasound and a sonosensitizer as described herein reduces or eliminates the need for one or more additional complementary treatments. For example, lower doses or fewer additional treatments of chemotherapy, radiation, cell therapy, etc. may be needed when cancerous tissue is treated by the combination of ultrasound and a sonosensitizer as described herein, thus enhancing patient care, and reducing side effects.

Several embodiments described herein administer the ensonification patterns in a manner that appropriately optimizes sonosensitizer activation and establish and/or deliver array technologies that can provide the appropriate accompanying broad sonosensitizer activation into a therapy. Several embodiments treat types of cancers that are difficult to surgically remove, as well as types that suffer from high reoccurrence. For example, glioblastoma (GBM), a Grade IV (i.e., highly aggressive) diffuse astrocytic glioma, is the most frequent and lethal type of brain cancer. Despite aggressive multimodal treatment at the time of diagnosis, the median overall survival for glioblastoma is approximately 1 year, and 5-year survival rates are only 10%. The pattern of recurrence in glioblastoma highlights the limitations of current treatments in targeting and removing all cancer cells. Cancers such as glioblastoma have no clear margins and therefore surgical removal of the cancer cells is almost impossible, as finger-like tentacles undetectably extend into surrounding healthy tissue. In several embodiments, the compositions, devices, and systems described herein are used to treat glioblastoma, as well as other tumors (both brain tumors and outside the brain). In various embodiments, cancers and tumors for sonodynamic treatment including, for example, hepatic cancer cells, murine sarcoma, leukemia, myeloid leukemia, cholangiocarcinoma, melanoma, squamous cells, osteosarcoma, gliosarcoma, astrocytoma, hepatocellular carcinoma, prostate, nephroblastoma, adenocarcinoma, gynecological, and other cancers. Gliomas, glial cells and/or astrocytomas are treated (e.g., selectively or preferentially) in several embodiments.

In several embodiments, one or more of the following features are provided: ensonification patterns that optimize activation of the sonosensitizer; ensonification patterns that adequately saturate a large treatment volume to ensure extraneous cancer cells in surrounding tissue are also treated; ensonification patterns and transducer array approaches that reduce or avoid hazards of coordinating and steering coherently focused energy in a manner that requires MRI or other imaging guidance, diagnostics, and/or monitoring, as these systems are untenable for delivering office-based therapies such as sonodynamic therapy, according to one embodiment. In some embodiments, however, MRI or other imaging guidance, diagnostics, and/or monitoring are used in conjunction with the devices described herein. In several embodiments, sonodynamic therapy is performed as a non-invasive office-based treatment (e.g., oncology clinic) for cancer. In one embodiment, a sonodynamic therapy treatment plan includes multiple repeat treatments of sonodynamic therapy over a time span of weeks (very similar to chemotherapy). The sonodynamic therapy benefits over other cancer therapies would include one or more of the following: minimal to no side effects, the sonosensitizer class of drugs are affordable naturally occurring compounds, efficient outpatient treatment regimen, and complimentary to other treatment options. In one embodiment, one or more sonosensitizers (such as 5-aminolevulinic acid (5-ALA)) is administered (e.g., orally) to a patient without imaging the location of the sonosensitizer(s) or its metabolites and/or products (such as protoporphyrin IX (PpIX)) for, e.g., tumor location purposes. In one embodiment, one or more sonosensitizers (such as 5-ALA) is administered (e.g., orally) to a patient without using the sonosensitizer(s) or its metabolites and/or products (such as PpIX) for diagnostic purposes (e.g., the administration of 5-ALA is therapeutic only).

In one embodiment, the present disclosure provides an ultrasound transducer for activating a sonosensitizer in conjunction with providing sonodynamic therapy. The ultrasound transducer comprises a plurality of ultrasonic transducer elements arranged in an array configured to generate a normalized, randomized, and/or incoherent acoustic pressure field with an energy profile for activating a sonosensitizer located within tissue of a patient.

In some embodiments, ultrasound is used for treatment in conjunction with a compound (e.g., drug), such as for treating cancer, neurological disease, mood condition, sleep apnea, inflammation and orthopedic diseases, and for opening the blood brain barrier. In some embodiments, ultrasound is used to activate a drug, pro drug, sonosensitizer, and/or microbubble additives. In some embodiments, ultrasound is combined with other energy (e.g., sonic, light, ultraviolet, infrared, electric, magnetic, electromagnetic, radiofrequency, and other forms of energy). In some embodiments, systems are used for treatments including cancer, neurological disease (e.g., Alzheimer's and others), mood condition, sleep apnea, inflammation, and/or orthopedic diseases, and opening the blood brain barrier to improve access to the drugs and additives. In various embodiments, ultrasound transducer systems are used with a cooling system, an alignment device (e.g., marking system, fiducial marks, magnetic tracking, automated alignment devices, imaging system, etc.) a monitoring system (e.g., temperature monitor, reflection monitor, cavitation monitor, imaging device) and/or a treatment planning system (e.g., imaging scan data via camera, CT, MRI, simulation software).

In various embodiments, an ultrasound transducer system configured to produce a normalized acoustic pressure profile for activating a sonosensitizer in conjunction with providing sonodynamic therapy, includes: an alignment device; and at least one ultrasound array, the at least one ultrasound array comprising a plurality of piezoelectric ultrasonic transducer elements configured to generate a normalized acoustic pressure profile for activating a sonosensitizer located within a tissue of a diseased organ of a patient, wherein the normalized acoustic pressure profile comprises: a peak pressure in a volumetric field; and an average acoustic pressure across the volumetric field.

In one embodiment, the normalized acoustic pressure profile is configured to minimize (e.g., reduce) a difference between the peak pressure and the average acoustic pressure in the volumetric free field, wherein the normalized acoustic pressure profile is configured to produce a peak pressure in the volumetric free field that is in a range of 101%-400% (e.g., 101%, 110%, 125%, 140%, 150%, 160%, 175%, 190%, 200%, 250%, 300%, 400%, 101%-200%, 125%-175%, 140%-190%, 101%-175%, 101%-175%, 101%-200%, 101%-300%, 101%-350%, 101%-390%, including ranges and values therein) of the average acoustic pressure in the volumetric field. In one embodiment, the normalized acoustic pressure profile is configured to minimize or reduce a difference between the peak pressure and the average acoustic pressure in the volumetric free field, wherein the normalized acoustic pressure profile is configured to produce a peak pressure in the volumetric free field that is in a range of 50-300% (e.g., 50%-99%, 50-75%, 60%-90%, 60%-80%, 70-95%, 70-80%, 101%-200%, 101%-300%, etc.) of the average acoustic pressure in the volumetric field.

In one embodiment, the normalized acoustic pressure profile is configured to optimize a relationship between the peak pressure and the average acoustic pressure in the volumetric free field. In one embodiment, the normalized acoustic pressure profile is configured to provide for a flat profile for the average pressure in the volumetric free field, wherein the normalized acoustic pressure profile is flattened to produce an average pressure across the volumetric free field that is in a range of 10-200% (e.g., 50%-99%, 20%-75%, 50%-90%, 60%-80%, 70%-95%, 70%-80%, 50%-150%, 50%-175%, 50%-200%, 100%-200%, 101%-200%, 110%-200%, 120%-200%, 125%-175%, 150%-200%, etc.). In one embodiment, the normalized acoustic pressure profile is configured to provide for a consistent average pressure in the volumetric free field, wherein the normalized acoustic pressure profile is configured to produce an average pressure across an incoherent volumetric free field that is in a range of 10-200% (e.g., 50%-99%, 20%-75%, 50%-90%, 60%-80%, 70%-95%, 70%-80%, 50%-150%, 50%-175%, 50%-200%, 100%-200%, 101%-200%, 110%-200%, 120%-200%, 125%-175%, 150%-200%, etc.). In one embodiment, the normalized acoustic pressure profile is configured to provide for a flat profile for the peak pressure in the volumetric free field, wherein the normalized acoustic pressure profile is flattened to produce an incoherent peak pressure across the volumetric free field that is in a range of 10-250% (e.g., 50%-99%, 20%-75%, 50%-90%, 60%-80%, 70%-95%, 70%-80%, 50%-150%, 50%-175%, 50%-250%, 100%-250%, 101%-200%, 110%-200%, 120%-200%, 125%-175%, 150%-250%, etc.). In one embodiment, the normalized acoustic pressure profile is configured to provide for a consistent peak pressure in the volumetric free field, wherein the normalized acoustic pressure profile is configured to produce a peak pressure across an incoherent volumetric free field that is in a range of 10-250% (50%-99%, 20%-75%, 50%-90%, 60%-80%, 70%-95%, 70%-80%, 50%-150%, 50%-175%, 50%-250%, 100%-250%, 101%-200%, 110%-200%, 120%-200%, 125%-175%, 150%-250%, etc.).

In various embodiments, the plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave, wherein the acoustic wave is a defocused acoustic wave or an unfocused acoustic wave. In one embodiment, an unfocused acoustic wave comprises a planar acoustic wave. In various embodiments, a defocused acoustic wave, a substantially defocused acoustic wave, a planar acoustic wave, substantially planar acoustic wave, unfocused acoustic wave, substantially unfocused acoustic wave, zero vergence acoustic wave, substantially zero vergence acoustic wave may be employed. The alignment device can be configured to align the tissue of the diseased organ of the patient with the at least one ultrasound array. In one embodiment, the volumetric field is generated with a normalized acoustic pressure profile to produce a volume that is within −2 dB to −15 dB (e.g., −2, −3, −4, −5, −6, −7, −8, −9, −10, −11, −12, −14, and −15 dB) of the peak pressure. In one embodiment, the volumetric field is generated to produce a volume that is within −2 dB to −15 dB which corresponds to a pulse average of 1-20 W/cm² (e.g., 1-18, 1-10, 1-15, 2-15, 2-10, 3-15, 5-15, 10-15 W/cm², and ranges and values therein) across a large therapeutic volume.

In varies embodiments, a normalized acoustic pressure profile is configured to provide for a consistent average pressure in the volumetric free field, wherein the normalized acoustic pressure profile is configured to produce an average pressure across an incoherent volumetric free field that is in a range of 10-200%. A random phase drive pattern can be configured to be filtered through a free field measurement to identify a phase drive pattern and a frequency drive pattern that attenuates peak pressure locations and results in a substantially uniform peak and average pressures throughout an incoherent pressure volume. A random phase drive pattern can be configured to be filtered through numerical simulations to identify a phase drive pattern and a frequency drive pattern that results in a substantially uniform peak and average pressures throughout an incoherent pressure volume. A random phase drive pattern can be configured to be filtered to remove patterns that produce an unintended coherence between one or more elements, thereby attenuating peak pressure locations to result in a more uniform field that can be driven to produce a larger therapeutic volume. In various embodiments, a unique drive signal is provided to each element for a duration of a single pulse, and is then alternated to a new unique combination for a subsequent pulse. A control algorithm can be configured to produce a unique phase drive pattern for each element in the at least one ultrasound array. In various embodiments, the acoustic wave is amplitude modulated, frequency modulated, phase modulated, continuous, discontinuous, pulsed, randomized, or combinations thereof. The random phase drive pattern can be configured to create an incoherent field. A normalized acoustic pressure profile can be produced by at least one unique phase combination generated by a control algorithm to produce a uniform peak pressure and an average pressure across an incoherent ultrasound field in order to increase a treatment volume of the volumetric free field. The control algorithm can be selected from a list of pre-screened phase sets, hydrophone measurements, simulations, or an analysis of disorder. In one embodiment, a volume of the volumetric free field is maximized.

In various embodiments, a controller is set to limit application of ultrasound energy only by interleaving successive sub-aperture bursts wherein the sub-apertures selected are designed to minimize sonication through hot spots. The controller can be set to modify applied phases on un-masked elements to minimize a delivered energy through hot spots. In various embodiments, apodization is applied to shift heat generation across an entry beam diameter.

In one embodiment, the normalized acoustic pressure profile further includes: a maximum pulse average acoustic pressure across the volumetric field; and a maximum pulse average acoustic pressure for a single plane in the volumetric field. The normalized acoustic pressure profile can be configured to activate the sonosensitizer, and/or the normalized acoustic pressure profile is configured to activate a drug, and/or the normalized acoustic pressure profile is configured to activate a microbubble additive. The system can include a cooling system a coupling membrane configured to conform to an anatomical feature of the patient and remove excess heat via circulation of a cooling fluid, wherein the cooling system comprises at least one pump to circulate the cooling fluid at a cooling rate in a range of 10-50 liters per minutes (e.g., 20-40 liters per minute). In one embodiment, an alignment device comprises a laser attached to a housing of the at least one ultrasound array, wherein the laser is attached to a targeting system configured to locate and verify a position of an alignment feature of an anatomical landmark on the patient for alignment of the sonodynamic therapy with the tissue of the diseased organ of the patient. The alignment device can include a robotic arm attached to a housing of the at least one ultrasound array, wherein the robotic arm is configured to position the at least one ultrasound array with an alignment feature of an anatomical landmark on the patient for alignment of the sonodynamic therapy with the tissue of the diseased organ of the patient. The alignment device can include a camera attached to a housing of the at least one ultrasound array, wherein the camera is attached to an imaging system configured to locate and verify a position of an alignment feature of an anatomical landmark on the patient for alignment of the sonodynamic therapy with the tissue of the diseased organ of the patient. In one embodiment, an imaging system comprises a monitor for displaying an image of the tissue of the diseased organ of the patient. The imaging system can include a monitor for displaying an image of an exterior surface of a body of the patient proximate the tissue of the diseased organ of the patient. In one embodiment, the imaging system comprises a computer learning system configured to use artificial intelligence to identify an exterior surface of a body of the patient proximate the tissue of the diseased organ of the patient, wherein the exterior surface of the body is calculated for an optimal treatment site. The alignment device can include at least one fiducial mark as an alignment feature of an anatomical landmark on the patient. In one embodiment, the alignment device comprises at least one magnetic tracking device as an alignment feature of an anatomical landmark on the patient. In one embodiment, the ultrasound transducer system includes a motorized alignment system attached to the at least one ultrasound array, wherein the motorized alignment system is configured to align a position of the at least one ultrasound array in one-dimension, two-dimensions, or three-dimensions. In one embodiment, the motorized alignment system comprises a track and a gimbal for controlled mechanical alignment of the at least one ultrasound array within a housing, wherein the housing is configured for attachment to the patient. In one embodiment, the alignment device comprises a custom 3D printed interface configured to attached to the patient. In one embodiment, the alignment device comprises a custom 3D printed interface configured to attached to the patient, wherein the custom 3D printed interface comprises a plurality of modular attachments for customized placement of the plurality of piezoelectric ultrasonic transducer elements in the at least one ultrasound array. In one embodiment, the system includes a cavitation monitoring device configured to modulate the average acoustic pressure across the volumetric field upon detection of a degree of cavitation, wherein the average acoustic pressure is increased to meet a minimum cavitation threshold. In one embodiment, the system includes a cavitation monitoring device configured to modulate the peak pressure across the volumetric field upon detection of a degree of cavitation, wherein the peak pressure is decreased to stay below a maximum cavitation threshold. In one embodiment, the system includes a passive cavitation monitoring device configured to modulate the average acoustic pressure across the volumetric field upon detection of a degree of cavitation. In one embodiment, the system includes a closed loop cavitation monitoring device configured to modulate the average acoustic pressure across the volumetric field upon detection of a degree of cavitation. In one embodiment, wherein the sonodynamic therapy is configured to open a blood brain barrier, treat a cancer, a nerve, Alzheimer's, Parkinson's disease, prion disease, multiple sclerosis, atherosclerosis, or sleep apnea.

In various embodiments, an ultrasound transducer system configured to produce a normalized acoustic pressure profile for activating a sonosensitizer in conjunction with providing sonodynamic therapy includes a plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in each ultrasound array comprises a planar emitting surface configured to emit a planar acoustic wave.

In some embodiments, the housing comprises a custom 3D printed interface configured to attached to the patient, and/or the housing comprises a custom 3D printed interface configured to attached to the patient, wherein the custom 3D printed interface comprises a plurality of modular attachments for customized placement of the plurality of piezoelectric ultrasonic transducer elements in each ultrasound array.

In various embodiments, an ultrasound transducer system configured to monitor cavitation to produce a normalized acoustic pressure profile for activating a sonosensitizer in conjunction with providing sonodynamic therapy includes: a cavitation monitoring device; and at least one ultrasound array, the at least one ultrasound array comprising a plurality of piezoelectric ultrasonic transducer elements configured to generate a normalized acoustic pressure profile for activating a sonosensitizer located within a tissue of a diseased organ of a patient. In one embodiment, the plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave, wherein the cavitation monitoring device configured to modulate the average acoustic pressure across the volumetric field upon detection of a degree of cavitation.

In various embodiments, the cavitation monitoring device is configured to increase the average acoustic pressure to a minimum cavitation threshold, and/or the cavitation monitoring device is configured to decrease the average acoustic pressure below a maximum cavitation threshold. In one embodiment, the cavitation monitoring device is passive and/or the cavitation monitoring device operates within a closed loop.

In various embodiments, an ultrasound transducer system configured to monitor reflected acoustic energy to produce a normalized acoustic pressure profile for activating a sonosensitizer in conjunction with providing sonodynamic therapy includes: a reflected acoustic energy monitoring device; and at least one ultrasound array. In one embodiment, a plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave, wherein the reflected acoustic energy monitoring device configured to modulate the average acoustic pressure across the volumetric field upon detection of a degree of reflected acoustic energy.

In one embodiment, the reflected acoustic energy monitoring device is configured to measure reflected power and increase the average acoustic pressure to a minimum reflected power threshold. In one embodiment, the reflected acoustic energy monitoring device is configured to measure reflected power and decrease the average acoustic pressure below a maximum reflected power threshold. In one embodiment, the reflected acoustic energy monitoring device is configured to measure reflected frequency and increase the average acoustic pressure to a minimum reflected frequency threshold. In one embodiment, the reflected acoustic energy monitoring device is configured to measure reflected frequency and decrease the average acoustic pressure below a maximum reflected frequency threshold. In one embodiment, the reflected acoustic energy monitoring device is passive. In one embodiment, the reflected acoustic energy monitoring device operates within a closed loop.

In various embodiments, an ultrasound transducer system configured to monitor reflected acoustic energy to produce a normalized acoustic pressure profile for activating a sonosensitizer in conjunction with providing sonodynamic therapy includes: a reflected acoustic energy monitoring device; at least one ultrasonic array, the at least one ultrasonic array comprising a plurality of piezoelectric ultrasonic transducer elements configured to generate a normalized acoustic pressure profile for activating a sonosensitizer located within a tissue of a diseased organ of a patient; a patient interface to acoustically couple the at least one ultrasonic array to the patient; and a controller coupled to the at least one ultrasonic array, wherein the controller is configured to: select one of the plurality of piezoelectric ultrasonic transducer elements of the at least one ultrasonic array for a reflected acoustic energy monitoring measurement; generate an ultrasound pulse with the one of the plurality of piezoelectric ultrasonic transducer elements; detect a reflection of the ultrasound pulse on the plurality of piezoelectric ultrasonic transducer elements of the ultrasonic array; and set amplitude and frequency of the one of the plurality of piezoelectric ultrasonic transducer elements based on the reflection; wherein the normalized acoustic pressure profile comprises: a peak pressure in a volumetric field; and an average acoustic pressure across the volumetric field; wherein the normalized acoustic pressure profile is configured to minimize or reduce a difference between the peak pressure and the average acoustic pressure in the volumetric field, wherein the normalized acoustic pressure profile is configured to produce a peak pressure in the volumetric field that is in a range of a range of 101%-400% (e.g., 101%, 110%, 125%, 140%, 150%, 160%, 175%, 190%, 200%, 250%, 300%, 400%, 101%-200%, 125%-175%, 140%-190%, 101%-175%, 101%-175%, 101%-200%, 101%-300%, 101%-350%, 101%-390%, including ranges and values therein) of the average acoustic pressure in the volumetric field; wherein the plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasonic array comprises a planar emitting surface configured to emit a planar acoustic wave, wherein the reflected acoustic energy monitoring device configured to modulate the average acoustic pressure across the volumetric field upon detection of a degree of reflected acoustic energy.

In one embodiment, the controller is configured to compute a minimum distance from the one of the plurality of elements to the tissue of the patient based on the reflections of the ultrasound pulse. In one embodiment, the minimum distance is a distance from the one of the plurality of elements to the tissue, and wherein the controller is further configured to compute a tissue thickness based on the reflections of the ultrasound pulse. In one embodiment, the controller is configured to compare a tissue thickness computed by the controller to a corresponding tissue thickness ascertained from imaging data of the tissue. In one embodiment, the setting of the amplitude and frequency by the controller is based on at least one of a minimum distance and a tissue thickness. In one embodiment, the controller is configured to optimize an ultrasound transmission rate through the patient based on a predetermined threshold. In one embodiment, the controller is configured to further set the amplitude and frequency of the one of the plurality of elements based on the reflections to minimize tissue heating of the patient during the sonodynamic therapy. In one embodiment, a suitable minimum tissue heating is ascertained based on a predetermined threshold.

In various embodiments, an ultrasound transducer system configured to monitor an acoustic parameter with an imaging device to produce a normalized acoustic pressure profile for activating a sonosensitizer in conjunction with providing sonodynamic therapy includes: an imaging device configured to monitor an acoustic parameter; at least one ultrasound array, the at least one ultrasound array comprising a plurality of piezoelectric ultrasonic transducer elements configured to generate a normalized acoustic pressure profile for activating a sonosensitizer located within a tissue of a diseased organ of a patient; a patient interface to acoustically couple the at least one ultrasonic transducer array to the patient; and a controller coupled to the at least one ultrasonic transducer array, wherein the controller is configured to: analyze an image of the tissue; and set amplitude and frequency of the one of the plurality of elements based on the imaging. In one embodiment the plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave, wherein the imaging device is configured to modulate the average acoustic pressure across the volumetric field upon detection of a degree of an acoustic parameter imaged by the imaging device.

In one embodiment, the controller is configured to compute a minimum distance from the one of the plurality of elements to the tissue of the patient based on the image. In one embodiment, the minimum distance is a distance from the one of the plurality of elements to the tissue, and wherein the controller is further configured to compute a tissue thickness based on the image. In one embodiment, the controller is configured to compare the tissue thickness computed by the controller to a corresponding tissue thickness ascertained from imaging data of the tissue. In one embodiment, the setting of the amplitude and frequency by the controller is based on at least one of the minimum distance and the tissue thickness. In one embodiment, the controller is configured to optimize the ultrasound transmission rate through the patient based on a predetermined threshold. In one embodiment, the controller is configured to further set the amplitude and frequency of the one of the plurality of elements based on the image to minimize tissue heating of the patient during the sonodynamic therapy. In one embodiment, a suitable minimum tissue heating is ascertained based on a predetermined threshold.

In various embodiments, an ultrasound transducer system configured to produce a normalized acoustic pressure profile for activating a sonosensitizer in conjunction with providing sonodynamic therapy includes: at least one ultrasound array, the at least one ultrasound array comprising a plurality of piezoelectric ultrasonic transducer elements configured to generate a normalized acoustic pressure profile for activating a sonosensitizer located within a tissue of a diseased organ of a patient. In one embodiment, a plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave.

In one embodiment, an acoustical output sensor is configured to measure an acoustical output of the at least one ultrasound array. The acoustical output sensor can be configured to measure an acoustical output from at least one ultrasonic transducer element in the plurality of piezoelectric ultrasonic transducer elements. The acoustical output sensor can be configured to measure an acoustical output from each ultrasonic transducer element in the plurality of piezoelectric ultrasonic transducer elements. In one embodiment, a power output sensor is configured to measure an power output of the at least one ultrasound array. The power output sensor can be configured to measure an acoustical output from at least one ultrasonic transducer element in the plurality of piezoelectric ultrasonic transducer elements. The acoustical output sensor can be configured to measure an acoustical output from each ultrasonic transducer element in the plurality of piezoelectric ultrasonic transducer elements. In one embodiment, an acoustical output pressure from the plurality of piezoelectric ultrasonic transducer elements have a value within a range of 0.1 MPa to 10 MPa across the at least one ultrasound array. In one embodiment, an acoustical output pressure from the plurality of piezoelectric ultrasonic transducer elements have a value within a range of 1% to 200% of each other across the at least one ultrasound array. In one embodiment, an acoustical output pressure from the plurality of piezoelectric ultrasonic transducer elements have a value within a range of 0.1 MPa to 10 MPa across the at least one ultrasound array across a range of treatment frequencies, In one embodiment, an acoustical output pressure from the plurality of piezoelectric ultrasonic transducer elements have a value within a range of 1% to 200% of each other across the at least one ultrasound array across a range of treatment frequencies. In one embodiment, a ratio of transducer element voltage to an acoustical output pressure from the plurality of piezoelectric ultrasonic transducer elements have a value within a range of 10%. In one embodiment, a drive parameter of the plurality of piezoelectric ultrasonic transducer elements is determined with a CT scan data, wherein the CT scan data comprises a tissue thickness of the patient. In one embodiment, the CT scan data further includes a thickness of a skull of the patient. In one embodiment, a drive parameter of the plurality of piezoelectric ultrasonic transducer elements is determined with a MRI scan data, wherein the MRI scan data comprises a tissue thickness of the patient. In one embodiment, the MRI scan data further includes a thickness of a skull of the patient. In one embodiment, a drive parameter of the plurality of piezoelectric ultrasonic transducer elements is determined with an acoustical simulation software to evaluate therapy parameters, wherein the acoustical simulation software simulates a tissue thickness of the patient.

In various embodiments, a method of producing the normalized acoustic pressure profile for enhancing an efficacy of the therapy uses the ultrasound transducer system, wherein the therapy is configured to open a blood brain barrier, treat a cancer, a nerve, Alzheimer's, Parkinson's disease, prion disease, multiple sclerosis, atherosclerosis, or sleep apnea.

In various embodiments, a method of producing a normalized acoustic pressure profile for enhancing an efficacy of a therapy configured to treat a diseased organ within an anatomical subject includes: generating, via a ultrasonic therapy system, a plurality of acoustic waves using at least one transducer array, wherein the at least one transducer array comprises a plurality of piezoelectric ultrasonic transducer elements; wherein the at least one transducer array is configured to generate a normalized acoustic pressure profile for activating a sonosensitizer located within tissue of a patient. In one embodiment, an immunotherapeutic effect is induced within the anatomical subject. In one embodiment, the immunotherapeutic effect comprises a resistivity to a recurrence of the diseased organ within the anatomical subject.

In various embodiments, a method of producing a normalized acoustic pressure profile for enhancing an efficacy of a sonodynamic therapy configured to treat a diseased organ within an anatomical subject includes: administering a sonosensitizing agent to the diseased organ within the anatomical subject; generating, via an ultrasonic therapy system, a plurality of acoustic waves using at least one transducer array, wherein the at least one transducer array comprises a plurality of piezoelectric ultrasonic transducer elements; wherein the at least one transducer array is configured to generate a normalized acoustic pressure profile for activating a sonosensitizer located within tissue of a patient, wherein the normalized pressure profile comprises: a peak pressure in a volumetric field; and an average acoustic pressure across the volumetric field; wherein the normalized pressure profile is configured to minimize or reduce a difference between the peak pressure and the average acoustic pressure in the volumetric field; activating, via the normalized pressure profile, a sonosensitizer within the diseased organ; and destroying, via the activation of the sonosensitizer, a diseased tissue in the diseased organ within the anatomical subject.

In one embodiment, the method includes identifying the tissue by measuring and marking a treatment site location on the patient with a ruler and writing device. In one embodiment, the method includes activating a laser alignment device to align the at least one transducer array with the tissue in the patient for treatment. In one embodiment, the method includes manually maneuvering for positioning the at least one transducer array with the tissue in the patient for treatment. In one embodiment, the method includes automated maneuvering for positioning the at least one transducer array with the tissue in the patient for treatment. In one embodiment, the method includes aligning the at least one transducer array with the tissue in the patient for treatment with at least one fiducial mark. In one embodiment, the method includes aligning the at least one transducer array with the tissue in the patient for treatment with computer vision. In one embodiment, the method includes aligning the at least one transducer array with the tissue in the patient for treatment with magnetic tracking. In one embodiment, the method includes coupling the at least one transducer array with the patient with a patient interface. In one embodiment, the patient interface is one or more selected from the group consisting of: a helmet, a mask, a neck brace, an arm sleeve, a glove, a mitten, a vest, a chest band, an abdominal band, a pelvic girdle, a leg sleeve, and a sock. In one embodiment, the sonosensitizing agent comprises 5-ALA and the sonosensitizer comprises protoporphyrin IX (PpIX). In one embodiment, the method includes administering a microbubble, wherein the microbubble is configured to enhance cavitation. In one embodiment, the method includes administering an oxygenating therapy configured to provide the tissue with supplemental oxygen. In one embodiment, the supplemental oxygen is provided to the tissue via a respiratory system of a patient. In one embodiment, the supplemental oxygen is provided to the tissue intravenously into a patient's bloodstream. In one embodiment, the supplemental oxygen therapy comprises a microparticle comprising supplemental oxygen, wherein the microparticle is configured to deliver the supplemental oxygen to the tissue. In one embodiment, the microparticles are specifically configured to target a specific location of a cell within the anatomical structure subject. In one embodiment, the oxygenating therapy comprises extracorporeal membrane oxygenation. In one embodiment, the extracorporeal membrane oxygenation comprises: removing a portion of a patient's blood; oxygenating the removed portion of blood with the supplemental oxygen; and introducing the oxygenated portion of blood back into the patient. In one embodiment, the oxygenating therapy comprises injecting the supplemental oxygen directly into a targeted tissue. In one embodiment, the oxygenating therapy comprises hyperbaric oxygen therapy. In one embodiment, the hyperbaric oxygen therapy comprises delivering oxygen to a cell at pressures above atmospheric pressure. In one embodiment, the supplemental oxygenating therapy comprises delivering a drug to enhance the oxygen concentration in a cell. In one embodiment, the drug comprises an antihypoxic drug configured to increase a level of oxygen in the cell. In one embodiment, the supplemental oxygenating therapy comprises reducing a metabolism of a cell, thereby reducing the rate at which oxygen is used by the cell and increasing the oxygen level within the cell. In one embodiment, the method includes monitoring a condition within the tissue using a cerebral oximeter. In one embodiment, the method includes monitoring an acoustic radiation force with imaging via CT or MRI. In one embodiment, the method includes monitoring changes in reflected power, frequency, or radiofrequency data.

In various embodiments, an ultrasound transducer system configured to produce a normalized acoustic pressure profile for activating a sonosensitizer in conjunction with providing sonodynamic therapy includes: an authorization system; and at least one ultrasound array, the at least one ultrasound array comprising a plurality of piezoelectric ultrasonic transducer elements configured to generate a normalized acoustic pressure profile for activating a sonosensitizer located within a tissue of a diseased organ of a patient. In one embodiment, a plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave, wherein the authorization device is configured to identify an identifier code on a drug, pro drug, sonosensitizer, and/or microbubble additive that has been administered to the patient, wherein the authorization device enables operation of the plurality of piezoelectric ultrasonic transducer elements if the identifier code matches an authorization code, wherein the authorization device disables operation of the plurality of piezoelectric ultrasonic transducer elements if the identifier code does not match the authorization code. In one embodiment, the identifier code is provided on any one of the group consisting of: an RFID, a bar code, a QR codes, and a hologram.

In various embodiments, a method of using acoustic waves for non-invasive ultrasound therapy to treat brain tumor cells includes: acoustically coupling an structure to a skin surface of a patient, the structure comprising: a shell, a flexible membrane, one or more imaging ultrasound transducer elements, and one or more treatment ultrasound transducer elements, wherein the flexible membrane defines a fluid filled cavity, wherein the flexible membrane is configured for conforming to the skin surface, wherein the flexible membrane is configured to acoustically couple the one or more imaging ultrasound transducer elements to the skin surface, wherein the flexible membrane is configured to acoustically couple the one or more treatment ultrasound transducer elements to the skin surface, driving the one or more treatment ultrasound transducer elements with a signal at a frequency to produce an acoustic wave in a treatment region to treat brain tumor cells, wherein each of the one or more treatment ultrasound transducer elements is configured to produce the acoustic wave; and circulating the fluid in the structure to facilitate acoustic coupling between the one or more treatment ultrasound transducer elements, the flexible membrane, and the skin surface. In various embodiments, ultrasound is transmitted through the skin surface transdermally or transcutaneously. In one embodiment, the driving the one or more treatment ultrasound transducer elements with the signal at the frequency to produce the acoustic wave in the treatment region to treat brain tumor cells comprising activating microbubbles with sound wave pressure.

In various embodiments, the ultrasound is provided at a frequency of 200-2000 kHz, 500-1500 kHz or 600-1200 kHz.

In various embodiments, methods of treating glioblastoma or other cancer in a brain include administering a microbubble agent to a patient, applying ultrasound to the brain of the patient, wherein such application of ultrasound temporarily opens a portion of a blood brain barrier, administering a chemotherapeutic agent and/or other anti-cancer agent, wherein said agent crosses the blood brain barrier through the opening created by the ultrasound application. In one embodiment, a method of treating glioblastoma or other cancer in a brain includes creating microbubbles in a patient, applying ultrasound to the brain, either through a skin surface or from within the brain, wherein such application of ultrasound temporarily opens a portion of a blood brain barrier, administering a chemotherapeutic agent and/or other agent, wherein said agent crosses the blood brain barrier through the opening created by said ultrasound application. Cavitation of microbubbles can temporarily open the blood brain barrier. Ultrasound to the brain can be delivered through a skin surface or from within the brain.

The agent can be one or more of the following: 5-aminolevulinic acid (5-ALA), protoporphyrin IX, hematoporphyrin, Rose Bengal, curcumin, titanium nanoparticles, chlorin e6, pheobromide-a, ATX-S10 (4-formyloximethylidene-3-hydroxy-2-vinyl-deuterio-porphynyl(IX)-6,7-dia spartic acid), photofrin, photofrin II, DCPH-P-Na(I), NPe6 (mono-I-aspartyl chlorin e6), polyhydroxy fullerenes, hypocrellin-B, ZnPcS2P2, methylene blue, sinoporphyrin sodium, a vitamin, tetracycline antibiotics (such as doxycycline, minocycline), deferoxamine, calcitriol, gefitinib, metformin, imiquimod, or methotrexate. The agent can include one or more of the following: Hexaminolevulinate (HAL), carmustine, temozolomide, paclitaxel, or carboplatin. The temporary opening of the blood brain barriers, in some embodiments, is reversible and for example can last less than a day, half day or even shorter such as 1-5 minutes, 5-120 minutes (e.g., 5, 10, 15, 20, 30, 45, 50, 60, 70, 75, 80, 90, 100, 110, 120 minutes and other values and ranges therein). In various embodiments, the opening of the blood brain barrier is accomplished through increased permeability or junction opening, for example. Opening of the blood brain barrier can be configured to allow access to certain agents and not others and is thus a selective opening of the blood brain barrier, wherein said selectivity is based on one or more of the following: type of agent, size of agent, molecular weight of agent, transporter associated with agent, or polarity of agent.

In various embodiments, a non-invasive method of damaging a mitochondria with a pro drug includes: administering an endogenous pro drug to a patient with cancer cells, wherein said pro drug comprises 5-aminolevulinic acid (5-ALA), transporting said 5-ALA through a cell membrane with an overexpression of peptide transporter 2 (PEPT2) resulting in increased production of protoporphyrin IX via a heme biosynthesis pathway, wherein said protoporphyrin IX is selectively accumulated in mitochondria in said cancer cells, activating said protoporphyrin IX via ultrasound, wherein said activating said protoporphyrin IX results in said protoporphyrin IX becoming cytotoxic thereby causing apoptosis of said cancer cells; and cooling said patient by circulating a cooling fluid around said patient.

In one embodiment, protoporphyrin IX is selectively accumulated in said cancer cells via a reduced expression of ferrochelatase. In one embodiment, protoporphyrin IX is selectively accumulated in mitochondria in said cancer cells. In one embodiment, activating said protoporphyrin IX via ultrasound comprises a sonodynamic therapy. In one embodiment, activating said protoporphyrin IX via ultrasound comprises a sonomechanical mechanism. In one embodiment, protoporphyrin IX converts dissolved molecular oxygen into a reactive oxygen species, and wherein activating said protoporphyrin IX via ultrasound comprises cavitation. In one embodiment, activating said protoporphyrin IX via ultrasound comprises causing thermal damage to said cancer cells, In one embodiment, administering said pro drug to said patient comprises an oral administration. In one embodiment, ultrasound is defocused or planar. In one embodiment, administering said pro drug to said patient comprises an injection or an intravenous administration. In one embodiment, administering a drug or pro drug to a patient comprises transdermal or transcutaneous delivery, such as via a patch, oral/sinus delivery, and/or sonophoresis. In one embodiment, ultrasound is used to disrupt a cell layer to transmit a drug or pro drug to a target tissue. In one embodiment, protoporphyrin IX is selectively accumulated in a glioblastoma multiforme (GBM) in a brain. In one embodiment, protoporphyrin IX is selectively accumulated in said cancer cells in a tissue selected from the group consisting of: a brain, a lung, a breast, a liver, a pancreas, an intestine, a stomach, a rectum, a vagina, testes, a prostate and a cervix. In one embodiment, activating said protoporphyrin IX produces an ablative treatment with a temporal average intensity without increasing a temperature of a healthy tissue in a treatment region above 42° C. In one embodiment, activating said protoporphyrin IX produces an ablative treatment with a pulse average intensity without increasing a temperature of a healthy tissue in a treatment region above 42° C.

In various embodiments, a non-invasive method of treating a mitochondria in a cancer cell with a pro drug includes: administering an endogenous pro drug to a patient with cancer cells, wherein said pro drug comprises 5-aminolevulinic acid (5-ALA), wherein said 5-ALA increases a heme biosynthesis pathway resulting in increased production of protoporphyrin IX, accumulating said protoporphyrin IX in mitochondria in said cancer cells as a result of reduced expression of ferrochelatase (FECH), activating said protoporphyrin IX with a sonodynamic treatment via ultrasound, wherein said activating said protoporphyrin IX results in said protoporphyrin IX becoming cytotoxic thereby causing necrosis of said cancer cells; and cooling said patient by circulating a cooling fluid around said patient.

In one embodiment, protoporphyrin IX is accumulated in said cancer cells via a reduced expression of ferrochelatase, In one embodiment, activating said protoporphyrin IX via ultrasound comprises a sonomechanical mechanism. In one embodiment, activating said protoporphyrin IX via ultrasound produces a reactive oxygen species. In one embodiment, administering said pro drug to said patient comprises an oral administration. Ultrasound is defocused or unfocused (e.g., planar) in some embodiments, but may be focused in other embodiments. In one embodiment, administering said pro drug to said patient comprises an injection or an intravenous administration.

### BRIEF DESCRIPTION OF THE DRAWINGS

The following drawings are illustrative of particular aspects (e.g., embodiments) of the present disclosure and therefore do not limit the scope of the appended claims. The drawings are intended for use in conjunction with the explanations in the following description. The disclosed embodiments will hereinafter be described in conjunction with the appended drawings, wherein like numerals denote like elements.

FIG. 1A is a block diagram of a general non-invasive ultrasound transducer system, according to at least one embodiment of the present disclosure.

FIG. 1B is a block diagram of a general non-invasive ultrasound transducer system with a temperature monitor, according to at least one embodiment of the present disclosure.

FIG. 1C is a block diagram of a general non-invasive ultrasound transducer system with a reflection monitor, according to at least one embodiment of the present disclosure.

FIG. 1D is a block diagram of a general non-invasive ultrasound transducer system with a cavitation monitor, according to at least one embodiment of the present disclosure.

FIG. 1E is a block diagram of a general non-invasive ultrasound transducer system with an imaging device, according to at least one embodiment of the present disclosure.

FIG. 1F is a block diagram of a general non-invasive ultrasound transducer system with an internal and/or external alignment device, according to at least one embodiment of the present disclosure.

FIG. 1G is a block diagram of a general non-invasive ultrasound transducer system with an authorization system, according to at least one embodiment of the present disclosure.

FIG. 1H is a block diagram of a general non-invasive ultrasound transducer system with one or more of a temperature monitor, reflection monitor, cavitation monitor, imaging device, internal alignment device, external alignment device, according to at least one embodiment of the present disclosure.

FIG. 2 is a schematic view of non-invasive ultrasound transducer systems placed in various locations about a patient's body according to embodiments of the present disclosure.

FIG. 3A is a perspective view of a transcranial sonodynamic therapy device with a housing having multiple transducers and a cooling system placed over the head of a patient, according to at least one embodiment of the present disclosure.

FIG. 3B is a perspective view of a transcranial ultrasound therapy system with multiple transducers and a cooling system placed over the head of a patient, according to at least one embodiment of the present disclosure.

FIG. 3C is a partial cutaway view of a transcranial ultrasound therapy system placed over the head of a patient showing a partial view of the multiple transducers, according to at least one embodiment of the present disclosure. is a partial cutaway view of a transcranial ultrasound therapy system placed over the head of a patient showing a partial view of the skull and brain of the patient and multiple transducers with one transducer emitting energy into the brain of the patient, according to at least one embodiment of the present disclosure.

FIGS. 4A-4C are schematic views of transducer arrays with multiple elements that can be individually energized to produce a variety of acoustic waves, including for example converging, diverging, and/or planar (zero vergence) acoustic waves, according to at least one embodiment of the present disclosure.

FIG. 5 is a diagram of two acoustic ultrasonic pulses without delay that constructively interfere, according to at least one embodiment of the present disclosure.

FIG. 6 is a schematic view of a patient interface with a cooling system with a fluid filled coupling membrane, according to at least one embodiment of the present disclosure.

FIG. 7 is a chart showing the relative sensitivity plot of an infrared (IR) temperature sensor, according to at least one embodiment of the present disclosure.

FIG. 8A is a block diagram of a general non-invasive sonodynamic therapy system, according to at least one embodiment of the present disclosure.

FIG. 8B is a graph illustrating a non-invasive sonodynamic therapy system with a filtering step to prescribe unique phasing for each element in an array for creating a large volumetric field according to at least one embodiment of the present disclosure.

FIG. 9 is an illustrative diagram of the sonodynamic therapy system, according to at least one embodiment of the present disclosure.

FIG. 10 is a schematic diagram of the sonodynamic therapy system, according to at least one embodiment of the present disclosure.

FIG. 11 is a schematic diagram of a sonodynamic therapy system with separate transmitting and receiving transducers, according to at least one embodiment of the present disclosure.

FIG. 12 is a schematic diagram of a sonodynamic therapy system with a single transmitting and receiving transducer, according to at least one embodiment of the present disclosure.

FIG. 13 is a diagram of a coherent drive field in accordance with at least one embodiment of the present disclosure.

FIG. 14 is a diagram of an incoherent field in accordance with at least one embodiment of the present disclosure.

FIG. 15A is a diagram of a pulsed therapy in accordance with at least one embodiment of the present disclosure.

FIG. 15B is a diagram of a pulsed therapy in accordance with at least one embodiment of the present disclosure.

FIG. 16 is a graph that illustrates a chirp signal, in accordance with at least one embodiment of the present disclosure.

FIG. 17 is a graph that illustrates an enveloped chirp signal, in accordance with at least one embodiment of the present disclosure.

FIG. 18 is a graph that illustrates a square ping signal, in accordance with at least one embodiment of the present disclosure.

FIG. 19 is a graph that illustrates a smooth ping signal, in accordance with at least one embodiment of the present disclosure.

FIG. 20 is a graph that illustrates an impulse signal, in accordance with at least one embodiment of the present disclosure.

FIG. 21 is a graph that illustrates an impulse input signal and first and second echoes with a time delay, in accordance with at least one embodiment of the present disclosure.

FIG. 22 is a logic flow diagram for monitoring an ultrasonic transducer array of a sonodynamic treatment system, in accordance with at least one embodiment of the present disclosure.

FIG. 23 is a logic flow diagram for monitoring an ultrasonic transducer array of a sonodynamic treatment system, in accordance with at least one embodiment of the present disclosure.

FIG. 24 is a logic flow diagram for monitoring an ultrasonic transducer array of a sonodynamic treatment system, in accordance with at least one embodiment of the present disclosure.

FIG. 25 is a flow diagram of a method of using a complementary and/or adjuvant therapy to enhance the efficacy of a sonodynamic therapy, according to at least one embodiment of the present disclosure.

FIG. 26 is a flow diagram of a method of using a supplemental oxygenating therapy to enhance the efficacy of a sonodynamic therapy, according to at least one embodiment of the present disclosure.

FIG. 27 is a flow diagram of a method of using immunotherapy to enhance the efficacy of a sonodynamic therapy, according to at least one embodiment of the present disclosure.

FIG. 28 is a block diagram of a block diagram depicting various therapeutic sonosensitizers configured to enhance the efficacy of a sonodynamic therapy, according to at least one embodiment of the present disclosure.

FIG. 29A is a schematic bottom view of an ultrasound transducer system according to at least one embodiment of the present disclosure.

FIG. 29B is a schematic isometric view of an ultrasound transducer system according to FIG. 29A.

FIG. 30 is a schematic side cross-section view the ultrasound transducer system of FIG. 29A.

FIG. 31 is an enlarged view of the section 31-31 denoted in FIG. 30.

FIGS. 32A-32F are schematic images of placements of an ultrasound transducer system at multiple locations around a head for overlapping treatment of tissue in a head according to at least one embodiment of the present disclosure.

FIGS. 33A-33J are schematic images of placements of an ultrasound transducer system at multiple locations around a head for overlapping treatment of tissue in a head according to at least one embodiment of the present disclosure.

FIGS. 34A-34B are schematic images of a targeting template with markers placed on a patient to facilitate alignment of the transducer to the various treatment sites according to various embodiments.

FIGS. 35A-35J are schematic images of placements of an ultrasound transducer system at multiple locations around a head with a targeting template according to at least one embodiment of the present disclosure.

FIGS. 36A-36C are schematic images of an embodiment of a sonodynamic treatment system with a transducer array, support arm, cart, console/controller, ultrasound generator, user interface, and/or a cooling fluid circulation unit.

FIG. 37 is illustrative of a schematic block diagram of an experimental set up for measurement of a normalized acoustic pressure profile to an area at a depth in tissue with various ultrasound transducer systems according to according to at least one embodiment of the present disclosure.

FIG. 38 is illustrative of the experiment in FIG. 37 with a schematic of a ultrasound transducer system configured for delivery of a normalized acoustic pressure profile to an area at a depth in tissue according to according to at least one embodiment of the present disclosure.

FIG. 39 is illustrative of a table of experimental measurements of delivery of a normalized acoustic pressure profile to an area at a depth in tissue with various ultrasound transducer systems according to FIG. 37.

FIGS. 40A-40B are illustrative of graphs of experimental measurements of delivery of a normalized acoustic pressure profile to an area at a depth in tissue with various ultrasound transducer systems according to FIG. 37.

FIG. 41 is illustrative of a graph of experimental measurements over time of delivery of a normalized acoustic pressure profile to an area at a depth in tissue with various ultrasound transducer systems according to FIG. 37.

FIG. 42 is illustrative of a graph of experimental measurements over time of delivery of a normalized acoustic pressure profile to an area at a depth in tissue with various ultrasound transducer systems according to FIG. 37.

FIG. 43 is illustrative of a sonodynamic treatment system with an ultrasonic transducer array according to at least one embodiment of the present disclosure.

FIGS. 44A-44B are illustrative of a helmet structure of the ultrasonic transducer array according to FIG. 43.

FIG. 45 is illustrative of a sonodynamic treatment system with a support arm of the ultrasonic transducer array according to FIG. 43.

FIG. 46 is illustrative of a sonodynamic treatment system with sub-apertures according to at least one embodiment of the present disclosure.

FIGS. 47A-47D are illustrative of a sonodynamic treatment system with a support arm of the ultrasonic transducer array according to FIG. 43.

FIGS. 48A-48C are illustrative of a sonodynamic treatment system with a helmet structure with the ultrasonic transducer array according to FIG. 43. FIG. 48A illustrates a top view of the helmet structure.

FIG. 48B shows a cross section of the helmet structure FIG. 48A. FIG. 48C shows a cross section of the helmet structure of FIG. 48B.

Like reference numbers represent corresponding parts or components throughout.

### DETAILED DESCRIPTION

The present disclosure relates, for example, to various embodiments of ultrasound therapy systems, such as non-invasive ultrasound. In several embodiments, an ultrasound transducer system for non-invasive therapy is provided that comprises, or consists essentially of, at least one transducer (e.g., at least one transducer array with a plurality of transducer elements), a patient interface to acoustically couple the transducer to a patient, and a controller coupled to the transducer(s). In several embodiments, the controller is configured to generate an electrical drive signal (e.g., one or more frequencies, phases, amplitudes, pulse widths, etc.) from a set of modulated acoustic wave parameters, modulate the drive signal, and drive the transducer with the modulated drive signal at a frequency to produce a normalized modulated acoustic wave to produce an acoustic intensity sufficient to treat tissue in a treatment region. In several embodiments, the ultrasound therapy system activates a sonosensitizer in a treatment region. In several embodiments, non-invasive therapy systems are not implanted in a patient. Minimally invasive systems are provided in other embodiments.

As disclosed in several embodiments herein, ultrasound transducer arrays are configured to generate ensonification drive patterns for initiating and enhancing therapeutic treatments with a normalized, randomized, and/or incoherent acoustic pressure field. In some embodiments, ultrasound is used for treatment alone, such as for treating cancer, neurological disease, mood condition, sleep apnea, inflammation and orthopedic diseases, and for opening the blood brain barrier. In some embodiments, ultrasound is used to activate a drug, pro drug, sonosensitizer, and/or microbubble additives. In some embodiments, ultrasound is combined with other energy (e.g., sonic, light, ultraviolet, infrared, electric, magnetic, electromagnetic, radiofrequency, and other forms of energy). In some embodiment, systems are used for treatments including cancer, neurological disease (e.g., Alzheimer's and others), mood condition, sleep apnea, inflammation, and/or orthopedic diseases, and opening the blood brain barrier to improve access to the drugs and additives. In various embodiments, ultrasound transducer systems are used with a cooling system, an alignment device (e.g., marking system, fiducial marks, magnetic tracking, automated alignment devices, imaging system, etc.) a monitoring system (e.g., temperature monitor, reflection monitor, cavitation monitor, imaging device) an authorization system (e.g., identification code, bar code, hologram for drug, key, and/or component for authorized operation of the system) and/or a treatment planning system (e.g., imaging scan data via camera, CT, MRI, simulation software).

In various embodiments, an ultrasound transducer system configured to produce a normalized acoustic pressure profile for activating a sonosensitizer in conjunction with providing sonodynamic therapy, includes: an alignment device; and at least one ultrasound array, the at least one ultrasound array comprising a plurality of piezoelectric ultrasonic transducer elements configured to generate a normalized acoustic pressure profile for activating a sonosensitizer located within a tissue of a diseased organ of a patient, wherein the normalized acoustic pressure profile comprises: a peak pressure in a volumetric field; and an average acoustic pressure across the volumetric field; wherein the normalized acoustic pressure profile is configured to minimize or reduce a difference between the peak pressure and the average acoustic pressure in the volumetric field, wherein the normalized acoustic pressure profile is configured to produce a peak pressure in the volumetric field that is in a range of 50-300% (e.g., 50%-99%, 50-75%, 60%-90%, 60%-80%, 70-95%, 70-80%, 101%-200%, 101%-300%, etc.) of the average acoustic pressure in the volumetric field; wherein the plurality of piezoelectric ultrasonic transducer elements is driven by one or more of: a modulated phase across the plurality of piezoelectric ultrasonic transducer elements, and a modulated frequency across the plurality of piezoelectric ultrasonic transducer elements, wherein each piezoelectric ultrasonic transducer element in the at least one ultrasound array comprises a planar emitting surface configured to emit a planar acoustic wave, wherein the alignment device is configured to align the tissue of the diseased organ of the patient with the at least one ultrasound array. In one embodiment, the volumetric field is generated with a normalized acoustic pressure profile to produce a volume that is within −2 dB to −15 dB (e.g., −2, −3, −4, −5, −6, −7, −8, −9, −10, −11, −12, −14, and −15 dB) of the peak pressure. In one embodiment, the volumetric field is generated to produce a volume that is within −2 dB to −15 dB which corresponds to a pulse average of 1-20 W/cm² (e.g., 1-18, 1-10, 1-15, 2-15, 2-10, 3-15, 5-15, 10-15 W/cm², and ranges and values therein) across a large therapeutic volume.

In one embodiment, the normalized acoustic pressure profile further includes: a maximum pulse average acoustic pressure across the volumetric field; and a maximum pulse average acoustic pressure for a single plane in the volumetric field. In various embodiments, the normalized acoustic pressure profile is configured to activate the sonosensitizer, and/or the normalized acoustic pressure profile is configured to activate a drug, and/or the normalized acoustic pressure profile is configured to activate a microbubble additive.

In several embodiments, a unique identifier is included on two or more of the following: a patient's wristband or other identification code; the ultrasound device; and the compound (e.g., 5-ALA) to ensure compatibility.

In various embodiments, methods and apparatuses for generating ultrasound ensonification drive patterns use ultrasound transducer arrays for initiating and enhancing therapeutic treatments in patients. Ultrasound therapy is a form of therapeutic treatment that uses ultrasound energy to treat tissue in patients. In some embodiments, ultrasound therapy is used alone for a treatment. In some embodiments, ultrasound therapy is used to activate a drug, prodrug, and/or sonosensitizer that selectively accumulates in target tissue cells.

In various embodiments, ultrasound is used to treat tissue. In various embodiments, tissue in the brain, lung, breast, colorectal region, prostate, bladder, ovary, testicle, pancreas, liver, stomach, intestine, colon, bone, spine may be treated using several embodiments described herein using for example, one or more ultrasound parameters described herein. In one embodiment, the ultrasound transducer system comprises an array with a first transducer, a second transducer, and a controller coupled to the first and second transducers. The controller is configured to generate a first electrical drive signal from a set of modulated acoustic wave parameters, generate a second electrical drive signal from the set of modulated acoustic wave parameters, drive the first transducer at the first electrical drive signal to produce a first acoustic wave, and drive the second transducer at the second electrical drive signal to produce a second acoustic wave. The first and second acoustic waves are combinable to produce an acoustic intensity sufficient to activate a sonosensitizer in a treatment region.

In one embodiment, an ultrasound transducer system comprises, or consists essentially of, a plurality of transducers and a controller coupled to the plurality of transducers. The controller is configured to generate a plurality of electrical drive signals from a set of modulated acoustic wave parameters and drive the plurality of transducers at the plurality of electrical drive signals to produce a plurality of modulated acoustic waves. The plurality of modulated acoustic waves is combinable to produce an acoustic intensity sufficient to activate a sonosensitizer in a treatment region.

In several embodiments, the ultrasound therapy is used to treat tissues, such as cancer tumors within the body, such as in the brain, spine, lung, breast, mouth, tongue, stomach, liver, pancreas, intestines, rectum, colon, vagina, ovary, testes, leukemia, lymphoma, among others, whether the tumors are malignant or nonmalignant. It will be appreciated, however, that such techniques can be applied to treat tumors or undesired tissue/cells within other body parts. For example, cancerous tissue in the lung, breast, colorectal region, prostate and pancreas may be treated using several embodiments described herein using for example, one or more sonosensitizers along with the ultrasound parameters described herein. Tumors that are difficult to access including those surrounded by bony structures are treated in various embodiments, including but not limited to spinal tumors. Treatment of undesired tissue in joints and other orthopedic applications are also provided herein. In some embodiments, the ultrasound therapy described herein can be used more invasively instead of or in conjunction with the noninvasive embodiments described here. Phototherapy using a light source may be used, in some embodiments, instead of or in conjunction with the ultrasound embodiments described here.

Sonodynamic therapy as defined herein shall be given its ordinary meaning and shall also include the therapeutic use of acoustic energy, including sound, sonic, and ultrasound energy to activate or enhance a process or substance (e.g., compound). Some non-limiting examples include invasive, noninvasive and/or minimally invasive therapeutic treatment that uses acoustic (e.g., sound, sonic, ultrasound) energy to activate a drug, prodrug, and/or sonosensitizer. These compounds can, for example, selectively accumulate in targeted tissue cells (e.g., cancer tumors, diseased tissue, organs, etc.). In one embodiment, the blood brain barrier may be opened via ultrasound sonication to deliver drugs (e.g., chemotherapy drugs such as carmustine, temozolomide, paclitaxel and/or carboplatin). In one embodiment, drugs can be delivered in a matrix, substrate, gel (e.g., hydrogel) form.

In one embodiment, 5-ALA results in the accumulation of PpIX in cancer through a mechanism involving having cancer cells preferentially transport 5-ALA through the cell membrane because of an overexpression of peptide transporter 2 (e.g., PEPT2). In one embodiment, 5-ALA results in the accumulation of PpIX in cancer through a mechanism involving having PpIX accumulate because cancer cells have reduced expression of ferrochelatase (FECH), which completes the synthesis of the heme group. Either of these two mechanisms, or a combination thereof, results in PpIX growing in concentration in tumor cells while remaining low in health cells.

In various embodiments, ultrasound in a sonodynamic treatment initiates one or more mechanisms of action. In one embodiment, the ultrasound excites PpIX to produce reactive oxygen species (ROS) and induce tumor cell death. In one embodiment, ROS excites PpIX via the release free oxygen radicals. In one embodiment, a target cell membrane is weakened by the sensitizer, and ultrasound pressure, radiation forces, and/or stable cavitational actions induce microstreaming and/or mechanical damage to the sensitized cell membrane. In one embodiment, stable cavitation creates mechanical and/or localized heat that damages the sensitized cells. In one embodiment, inertial cavitation creates sonoluminescence, and the localized light emission activates the PpIX to produce ROS. In one embodiment during sonodynamic therapy, PpIX behaves as a catalyst that converts molecular oxygen from a low-energy state into a higher energy state. These high-energy oxygen molecules are violently reactive and will damage cellular components. In particular, this reactive oxygen species (ROS) damages the mitochondria of cancer cells where the highest concentrations of PpIX occurs. In one embodiment, during sonodynamic therapy the PpIX is activated under a specialized low intensity therapeutic ultrasound regime. In one embodiment, the ultrasound pulses induce a cavitation environment, and subsequently the energy from the cavitation leads to activation of the PpIX. Damage to mitochondria in cancer cells or other undesired target tissue includes, for example, reduction in function, activity and/or structure to the mitochondria in a manner that impairs the cancer cell. In one embodiment, cancer cells are treated via induction of ferroptosis, such as cancel cell death triggered by removing or reducing certain amino acids. Ferroptosis is an iron-dependent type of programmed cell death that is effective in treating cancer, such as glioblastoma.

In one embodiment, a sonosensitizing agent (e.g., drug, prodrug, sonosensitizer) preferentially accumulates in the cells of the target tissue. In one embodiment, the sonosensitizing agent increases a quantity, accumulation, or concentration of a sonosensitizer in the cancer targeted tissue cells. Sonosensitizers, in some embodiments, initiate a cytotoxic response in target tissues when exposed to ultrasonic energy. Upon activation by the ultrasonic energy, sonodynamic therapy drugs or “sonosensitizers” produce reactive oxygen species (ROS) that generate the cytotoxic effect. Reactive oxygen species (ROS) as defined herein shall be given its ordinary meaning and shall also include free radicals, oxygen radicals, and/or unstable molecules that contain oxygen that react easily with other molecules. In some embodiments, ROS cause damage to proteins, DNA, RNA, and can cause cell death. In some embodiments, sonodynamic therapy is used herein to improve efficiency of chemotherapeutic molecules, sonoporation, and/or gene delivery. In various embodiments, sonodynamic therapy with an ultrasound array delivering a temporal-average intensity at the target site (e.g., tissue for treatment) of 0.1, 0.5, 1, 2, 3, 4, 5, 6, 7, 8, 9, 10, 11, 12, 13, 14, 15, 16, 17, 18, 19, 20 W/cm² (e.g., 0.1-8 W/cm² 0.1-4 W/cm², 0.5-5 W/cm², 1-20 W/cm², 2-15 W/cm², 3-12 W/cm², 5-15 W/cm², 5-10 W/cm², 10-15 W/cm², 7-12 W/cm², 8-13 W/cm² etc.) to cancer tissue can be used to induce and activate sonosensitizer at relative deep depths within a patient's body with or without cavitation and/or thermal effects and/or sonoluminescence to produce reactive oxygen species, intracellular singlet oxygen, and/or free radicals in a cascade of events that activate the sonosensitizer and in turn damage the cancer cells. In various embodiments, sonodynamic therapy with an ultrasound array delivering a pulse-average intensity at the target site (e.g., tissue for treatment) of 0.1, 0.5, 1, 2, 3, 4, 5, 6, 7, 8, 9, 10, 11, 12, 13, 14, 15, 16, 17, 18, 19, 20 W/cm² (e.g., 0.1-8 W/cm² 0.1-4 W/cm², 0.5-5 W/cm², 1-20 W/cm², 2-15 W/cm², 3-12 W/cm², 5-15 W/cm², 5-10 W/cm², 10-15 W/cm², 7-12 W/cm², 8-13 W/cm² etc.) to cancer tissue can be used to induce and activate sonosensitizer at relative deep depths within a patient's body with or without cavitation and/or thermal effects and/or sonoluminescence to produce reactive oxygen species, intracellular singlet oxygen, and/or free radicals in a cascade of events that activate the sonosensitizer and in turn damage the cancer cells. In various embodiments, sonodynamic therapy can be used with or without photodynamic therapy. In various embodiments, sonodynamic therapy with an ultrasound array delivering a temporal-average intensity output at the entry plane of 20, 40, 50, 75, 100, 125, 150, 175, 200, 225, 240, 250, 260, 275, 300, 350, 400 W/cm² (e.g., 20-400 W/cm², 100-300 W/cm², 200-300 W/cm², 150-250 W/cm², 200-250 W/cm², 250-300 W/cm² etc.) to cancer tissue can be used to induce and activate sonosensitizer at relative deep depths within a patient's body with or without cavitation and/or thermal effects and/or sonoluminescence to produce reactive oxygen species, intracellular singlet oxygen, and/or free radicals in a cascade of events that activate the sonosensitizer and in turn damage the cancer cells. In various embodiments, sonodynamic therapy with an ultrasound array delivering a pulse-average intensity output at the entry plane of 20, 40, 50, 75, 100, 125, 150, 175, 200, 225, 240, 250, 260, 275, 300, 350, 400 W/cm² (e.g., 20-400 W/cm², 100-300 W/cm², 200-300 W/cm², 150-250 W/cm², 200-250 W/cm², 250-300 W/cm² etc.) to cancer tissue can be used to induce and activate sonosensitizer at relative deep depths within a patient's body with or without cavitation and/or thermal effects and/or sonoluminescence to produce reactive oxygen species, intracellular singlet oxygen, and/or free radicals in a cascade of events that activate the sonosensitizer and in turn damage the cancer cells. In various embodiments, sonodynamic therapy can be used with or without photodynamic therapy.

Several embodiments described herein are used synergistically with other cancer therapies, including for example, radiation, chemotherapy and cell therapies. In one embodiment, the combination of ultrasound and a sonosensitizer as described herein reduces or eliminates the need for one or more additional complementary treatments. For example, lower doses or fewer additional treatments of chemotherapy, radiation, cell therapy, etc. may be needed when cancerous tissue is treated by the combination of ultrasound and a sonosensitizer as described herein, thus enhancing patient care and reducing side effects. In some embodiments, ultrasound therapy is used in conjunction with an auxiliary or additional energy (e.g., sonic, light, ultraviolet, infrared, electric, magnetic, electromagnetic, radiofrequency, and other forms of energy). In one embodiment, sonodynamic therapy may be performed in conjunction with autologous T-cell therapy (such as INB-400) and/or an allogeneic (such as INB-410) for the treatment of patients with newly diagnosed glioblastoma.

In some embodiments, ultrasound therapy is used to activate a drug, prodrug, sonosensitizer, and/or microbubble additive that selectively accumulates in cells within the tissue for treatment. In one embodiment, a sonosensitizing agent (e.g., drug, prodrug, sonosensitizer, microbubble additive) preferentially accumulates in the cells of tumors, lesions, damaged or affected tissue.

In various embodiments, ultrasound therapy is used to temporarily allow for increased access through a blood brain barrier for treatments such as sonodynamic therapy, chemotherapy, improved mindfulness and meditation, hallucinatory effects, and recreational enhancement. In various embodiments, increased access through the blood brain barrier allows for exchange of materials in to and/or out of the brain via blood in blood vessels. In one embodiment, ultrasound therapy with or without microbubbles are used to open the blood brain barrier to treat cancer in the brain, such as glioblastoma. Opening the blood brain barrier can increase transport of drugs to the brain by ˜300%-600%.

Applicant of the present application owns the following PCT International Patent Applications, the disclosure of each of which is herein incorporated by reference in its respective entirety: (1) PCT International Application No. PCT/US2015/010053 entitled Device and Method For Use Of Photodynamic Therapy filed Jan. 2, 2015, national phase now U.S. Pat. No. 10,675,482; (2) PCT International Application No. PCT/US2019/045802 entitled Tissue Treatment with Sensitizer and Light and/or Sound filed Aug. 8, 2019; (3) PCT International Application No. PCT/US2020/017983 entitled Non-Invasive Sonodynamic Therapy filed Feb. 12, 2020; and (4) PCT International Application No. PCT/US2021/071101 entitled Ultrasound Arrays For Enhanced Sonodynamic Therapy For Treating Cancer filed Aug. 4, 2021.

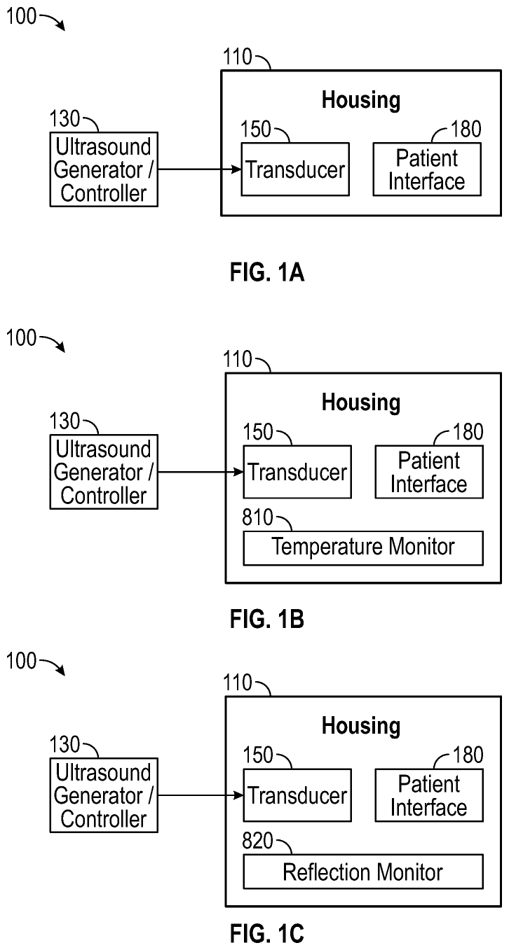

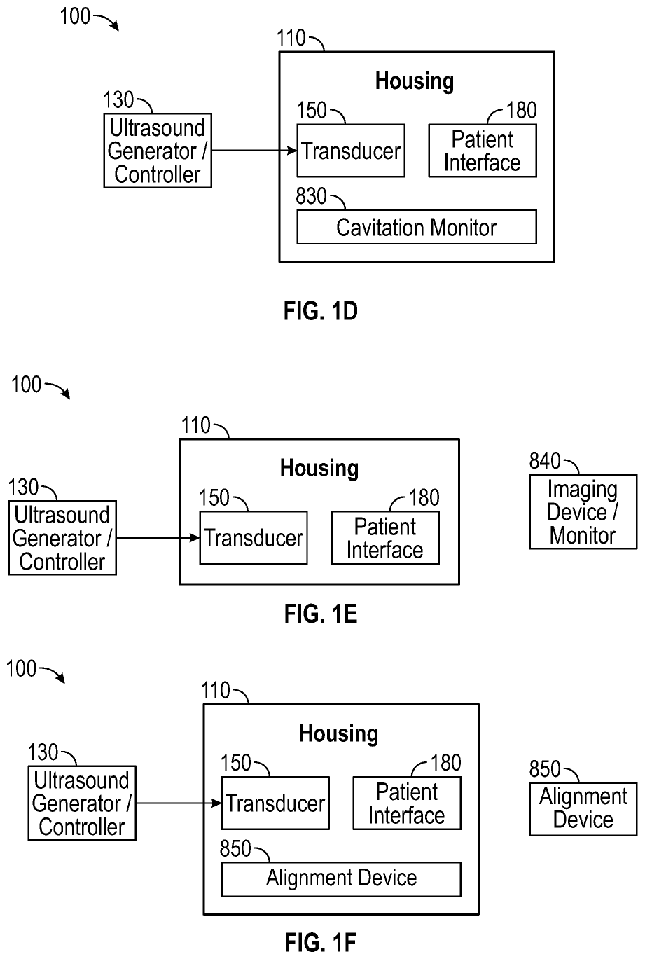

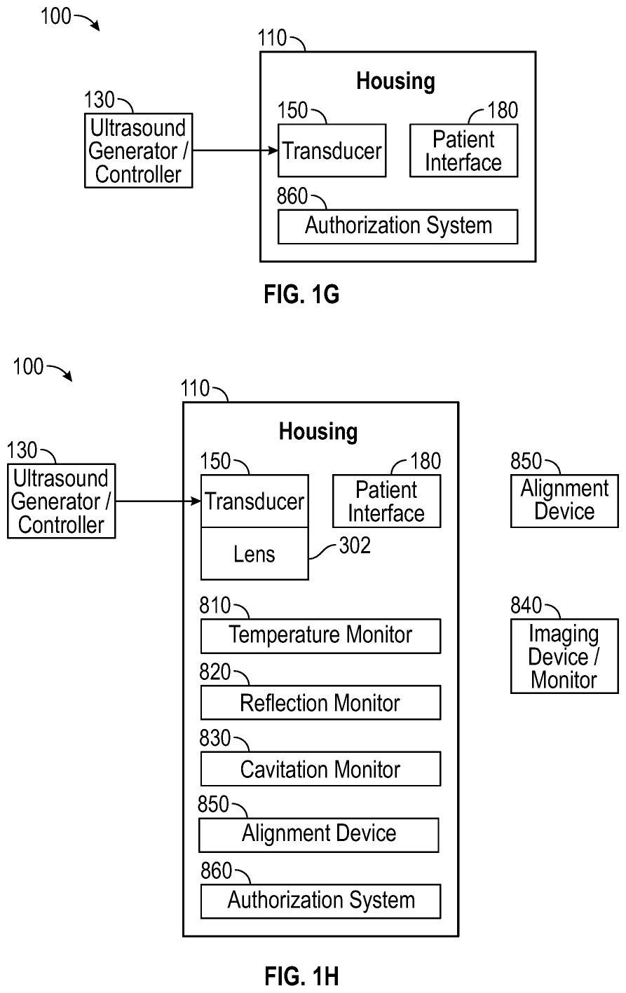

Turning now to FIG. 1A, an ultrasound transducer system 100 for non-invasive therapy comprises, or consists essentially of, at least one transducer (e.g., at least one transducer array with a plurality of transducer elements) 150, a patient interface 180 to acoustically couple the transducer to a patient, and an ultrasound generator and/or controller 130 that is coupled to a housing 110 and the transducer(s) 150.

In various embodiments, the ultrasound transducer system 100, controller 130, and transducers 150 are configured to produce a normalized, randomized, and/or incoherent acoustic pressure field to produce an acoustic intensity sufficient to treat tissue in a treatment region.

FIGS. 1B-1H illustrate embodiments of the ultrasound transducer system 100 in FIG. 1A with one or more of a temperature monitor 810 (FIGS. 1B and 1H), reflection monitor 820 (FIGS. 1C and 1H), cavitation monitor 830 (FIGS. 1D and 1H), imaging device 840 (FIGS. 1E and 1H), internal alignment device 850 and/or external alignment device 850 (FIGS. 1F and 1H), and/or authorization system 860 (FIGS. 1G and 1H), and combinations thereof.

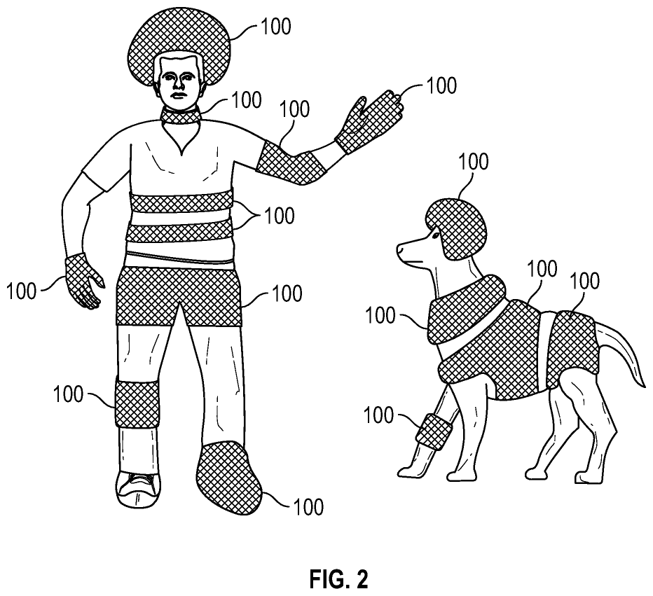

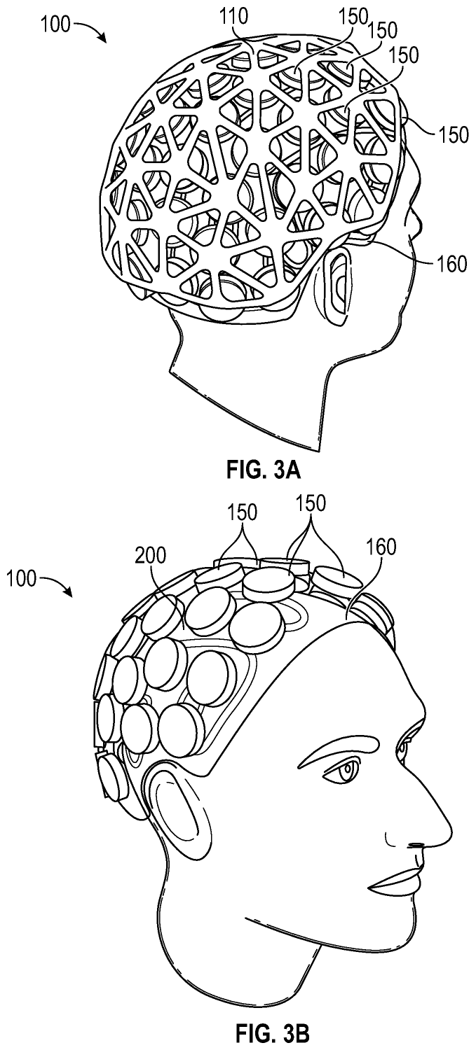

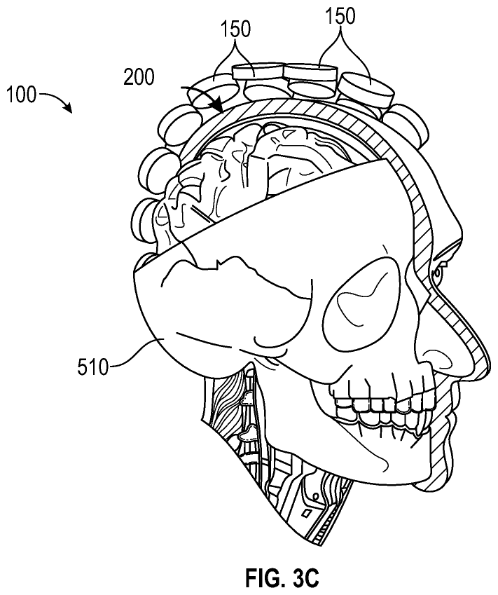

FIG. 2 illustrates embodiments of ultrasound transducer systems 100 comprising a patient interface 180 in the shape of a helmet, a cap, a headset, a mask, a neck sleeve, an arm sleeve, a glove, a mitten, a vest, a chest band, an abdominal band, a pelvic girdle, a leg sleeve, and/or a sock. In various embodiments, patients may include humans and animals (e.g., veterinary applications with pets, livestock, dogs, cats, rodents, horses, cattle, lambs, sheep, birds, reptiles, amphibians, fish, and others). In various embodiments, the ultrasound transducer system 100 may be used with any part of the patient's body. Certain examples herein illustrate embodiments for the head, however applications may be applied to any part of the patient's body. In several embodiments, multiple ultrasound transducer systems 100, patient interfaces 180, and/or arrays, transducers, or elements are positioned and oriented to accomplish full treatment (e.g., full patient, organ, and/or tissue coverage) without having to move to multiple discrete locations. In one embodiment, an entire organ is treated with one or more ultrasound transducer systems 100, patient interfaces 180, and/or arrays, transducers, or elements. Sonodynamic treatments of entire organs and larger tissue volumes are especially advantageous when using non-focused, normalized ultrasound because these treatments cover large areas. Other embodiments using converging focusing techniques involve specific targeting in an attempt to avoid over heating and excess damage to surrounding tissues. In one embodiment, an ultrasound transducer system (e.g., a non-invasive sonodynamic therapy device) 100 provides for treatment of a head as shown in FIGS. 3A-3C. Skull thicknesses and density can vary by gender, breed, and anatomical location. The non-invasive sonodynamic therapy device 100 may comprise a housing (e.g., a shell) with transducers that can provide predictable and consistent ensonification despite these variations. In one embodiment, the housing 110 may comprise a rigid material. in one embodiment, the housing 110 may comprise a flexible material, such as an elastic material that conforms to a portion of the patient's body surface. In various embodiments, the transducers 150 may be modularly attachable and detachable to the housing for custom placements. In various embodiments, the transducers 150 may be fixed to the housing. In various embodiments, a portion of all of the ultrasound transducer system 100 (e.g., housing 110, patient interface 180) may be customized (e.g., custom 3D printed or formed, sewn, etc.) for customized fit to a particular patient. In one embodiment, modular patient interfaces, arrays, transducers and/or transducer elements are configured for removable and customized placement with respect to the system and/or patient. Known relative positions of the transducers 150 can allow for imaging of the head, even in low resolution with transducers 150. In one embodiment, the illustrated embodiment may include a mobile stand to hold in position on the patient while he/she waits in a standing, seated, supine, prone, or other position. In one embodiment, the shell 110 may be a lightweight helmet that can be worn by the patient during treatment, allowing for predictable placement of the transducers 150 with little infrastructure requirements. In one embodiment, the shell 110 may be part of a positionable system attached to an arm and/or mobile stand.

The non-invasive sonodynamic therapy device 100 may comprise a flexible shell 110 (e.g., a helmet, cap, head sleeve) with transducers 150 placed over a liquid-cooled skull cap 160 as described further elsewhere herein, requiring, in several embodiments, little infrastructure to support the array of transducers 150. It may be possible for the patient to don the skull cap 160 and shell 110 in any chair while he/she waits for treatment to complete. The lightweight design may minimize neck pain from the patient holding up his/her head for extended periods with the weight of the transducers 150 and cooling cap. The flexible shell 110 can conform to the shape of each skull. Such a device may account for subtle variations between treatments depending on the shape of each patient's head curving some transducers 150 more inward or outward.

The non-invasive sonodynamic therapy device 100 may comprise rigid or flexible portions or patches with several transducers 150 that can be removably applied to the head. Such an embodiment may involve clinicians applying each modular patch individually. Having separate, modular patches (such as sub-arrays, elements or other components) can allow for some treatment customization and flexibility without requiring each transducer 150 to be planned and placed individually. In one embodiment, a non-invasive sonodynamic therapy device 100 may minimize sores caused by adhering patches to the head repeatedly, which may be a particular concern for older and sicker patients.

The non-invasive sonodynamic therapy device 100 may comprise patches with single transducers 150 that can be removable applied to the patient. Individual transducers 150 can provide the significant treatment flexibility. Such a device may involve a detailed process for planning to apply and applying the transducers 150.

The size and shape of the transducers 150, as can be seen in FIG. 3B, may vary across various disclosed embodiments. For a cost-effective and simple system, larger transducers 150, which produce directional acoustic waves, may be used. Large transducers 150 can be made less directional by applying to each transducer 150 an optional acoustic lens 302 that bends the acoustic waves as described further elsewhere herein. For a system that can conform to the head, smaller transducers 150, which can radiate more broadly than larger transducers 150, can be used. Such small transducers 150 can have a greater ability to image or beam steer as an array.

FIG. 3C is a partial cutaway view of a transcranial sonodynamic therapy device 100 placed over the head of a patient showing a partial view of the multiple transducers 150, according to at least one embodiment of the present disclosure. Instead of focusing an acoustic wave 200 to a small point, the acoustic wave 200 can be normalized to minimize the spatial variation of the acoustic wave intensity in the brain.

In various embodiments, the size and/or shape of the transducers 150 and/or one or more lenses 302 may defocus or focus each transducer 150. In one embodiment, a transducer 150 is used with a lens 302. In one embodiment, a transducer 150 is used without a lens 302. In one embodiment, no lens 302 is present. In one embodiment, a lens 302 is present. Focus and focused as defined herein shall be given its ordinary meaning and shall also include converging or convergent energy beams. Defocus and defocused as defined herein shall be given its ordinary meaning and shall also include diverging or divergent energy beams. Unfocus and unfocused as defined herein shall be given its ordinary meaning and shall also include energy beams that do not converge or diverge, such as with planar waves. In various embodiments, the size and shape of the transducer elements may defocus or focus each transducer element. As used herein (unless described otherwise), the term focused refers to a converging acoustic wavefront that is more convergent than a wavefront produced by a transducer 150 with a planar emitting surface and the term defocused refers to an acoustic wavefront that is more divergent than a wavefront produced by a transducer 150 with a planar emitting surface. Whether a lens 302 or the surface needs to be concave or convex to make a wave more divergent depends on whether the acoustic wave is transitioning from a region of low acoustic impedance to a region of high acoustic impedance or the acoustic wave is transitioning from a region of high acoustic impedance to a region of low acoustic impedance. In this regard, if a lens is made of a material with higher acoustic impedance than the target medium (water/tissue), the acoustic wave originates in the high-impedance material and transitions to the low-acoustic impedance target medium. In one embodiment, if the transducer or lens is concave, it will mechanically “focus” the acoustic wave to make it more convergent. In one embodiment, if the transducer or lens is convex, it will mechanically “defocus” the acoustic wave to make it more divergent.

In one embodiment, one or more lenses 302 (e.g., lensing) is applied to the transducer array to convert the natural focus of the elements of an array to a plane wave. Several embodiments herein overcome certain challenges and limitations in creating a large volume therapeutic ultrasound field. Disclosed are additional lensing approaches to create a desired large volume therapeutic ultrasound field. The lensing specifics are informed by element sizing, overall array sizing, shape, and geometry to result in a targeted plane wave output from each element in the array that promotes an overall larger therapeutic volume from the array. In one embodiment an inverse lens is applied to convert the natural focus of each element to a plane wave or a pseudo plane wave. In one embodiment, the inverse lens is thinner at the center and thicker at the element edges, in order to delay the outer element acoustic energy relative to the inner element energy. In one embodiment, the lens optimizes the array to produce an overall therapeutic field consisting of quasi plane waves distributed across a therapeutic volume. In one embodiment a slow lens, using a material with sound speed less than water that is concave (e.g., thinner at the center and thicker at the element edges) to defocus the element output. In one embodiment a fast lens is applied using a material with sound speed greater than water, such that the lensing effect creates a defocused element output that increases the overall therapeutic field volume emitted by the array. In various embodiments, lensing combinations and approaches effectively defocus each element to create a pseudo plane wave. This can be done using a lens, for example a silicone concave lens, per element. In one embodiment, this can be accomplished using a faceplate across the entire array surface employing combinations of lens types described herein.

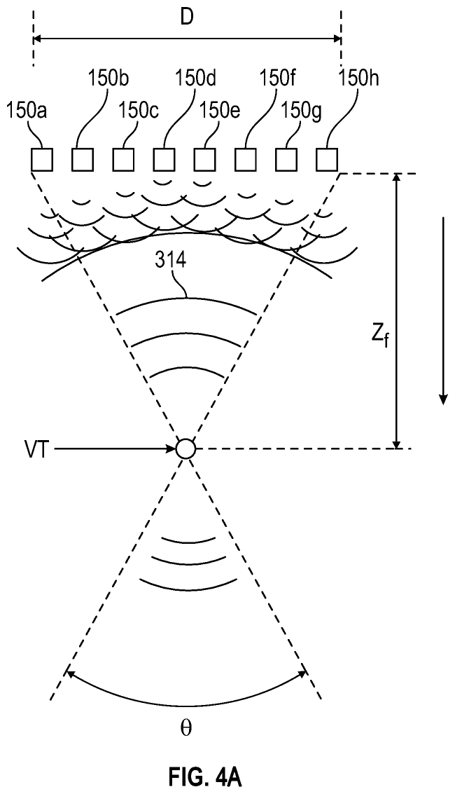

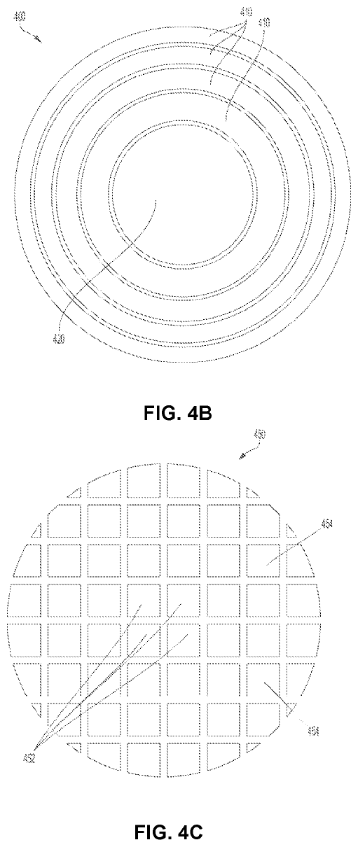

FIG. 4A is a schematic view of a transducer 150 with multiple elements 150a-150h (e.g., an array) that can be individually energized to produce a variety of acoustic waves, according to at least one embodiment of the present disclosure. As shown in FIG. 4A, multiple transducer elements 150a-150h can be arranged in an array to produce converging, diverging, or planar (zero vergence), acoustic waves. In one embodiment, one or more of the individual elements 150a-150h includes a flat, planar emitting surface that produces a planar acoustic wave. In various embodiments, one or more (e.g., 1, 5, 10, 20, 50, 100, 200, 250, 256, 300, 500, 1000, or more, and in some embodiments, all) elements of an array include flat planar emitting surfaces. In some embodiments, the array consists essentially of flat planar emitting surfaces ranging, for example, from 150-350 elements, 100-300 elements, 200-300 elements, 800-1200 elements, and values and ranges therein. In one embodiment, the array of flat, planar emitting surfaces is arranged on a flat array. In one embodiment, the array of flat, planar emitting surfaces is arranged with a curvature (e.g., on a curved surface) configured to direct each flat element in the array to emit the planar acoustic wave normal to a body surface, such as a skull, other bony structure. In some embodiments, the chest, neck, abdomen, back, waist, shoulder, or other body structure is treated. Ultrasound is defocused or unfocused (e.g., planar) in some embodiments, but may be focused in other embodiments.

In various embodiments, the focus, defocus, or planar/zero vergence emission of ultrasound waves of the transducers 150 can depend on the material and shape of the emitting surface of the transducer face and/or a supplemental lens 302. In one embodiment, the transducers 150 are flat, which may minimize manufacturing costs. Both the lens 302 with the concave surface and the lens 302 with the convex surface 310 may be configured to produce a fixed focus. In one embodiment, both the lens 302 with the concave surface and the lens 302 with the convex surface 310 may be configured to produce a fixed or beam broadening focus. In one embodiment, the lens 302 with the concave surface and the lens 302 with the convex surface 310 may be configured to produce a defocused beam.

In one embodiment, the transducer can adjust its shape to create different focuses using an elastic, fluid-filled pocket that functions as a lens. In one embodiment, a fluid-filled pocket is configured to converge an acoustic wave. In one embodiment, a fluid-filled pocket is configured to diverge an acoustic wave. In one embodiment, a fluid-filled pocket does not affect the convergence or divergence of an acoustic wave. In one embodiment, a fluid-filled pocket does not affect a planar acoustic wave. The fluid can be pumped in or out of the lens to adjust shape of the pocket and thus the focus of the transducers.

With reference to FIGS. 4A, 4B, and 4C, in various embodiments, the acoustic wave produced by the transducer 150, 400, 450 may be defined by vergence—a measure of the curvature of the acoustic wavefront. A negative vergence is when the acoustic wavefront propagates away from a point (e.g., divergence). A positive vergence is when the acoustic wavefront propagates towards a point (e.g., convergence). A zero vergence is a planar acoustic wavefront that does not converge or diverge. Vergence is a property of a single acoustic wavefront. In one embodiment, a single converging/diverging acoustic wavefront may be produced by multiple elements of a transducer 150, 400, 450 (e.g., a transducer comprising an annular array 400 or a grid array 450). In one embodiment, a converging/diverging acoustic wavefront may be produced by each individual element of a transducer 150, 400, 450 (e.g., a transducer comprising an annular array 400 or a grid array 450). In one embodiment, the transducers 150, 400, 450 may be adapted and configured to produce a“focused” acoustic wave by producing a convergent acoustic wave that converges to a point. In another embodiment, the transducers 150, 400, 450 may be adapted and configured to produce a “defocused” acoustic wave, e.g., a divergent acoustic wave. In other embodiments, the transducers 150, 400, 450 may be adapted and configured to produce a planar acoustic wave (e.g., zero vergence) where the acoustic wave is neither “focused” nor “defocused.”

In one embodiment, a transducer 150 has a concave emitting surface (or has a lens 302 defining a concave surface), according to at least one embodiment of the present disclosure. The lens 302 may be acoustically coupled to the transducer 150 or may be formed integrally therewith. In one embodiment, the lens 302 is made of a material with higher acoustic impedance than the target medium (water/tissue) such that the acoustic wave 200 originates in the high-impedance material and transitions to the low-acoustic impedance target medium causing the acoustic wave 200 “focus” or converge to the target tissue. In one embodiment, the lens 302 is made of a material with higher acoustic impedance than the target medium (water/tissue) such that the acoustic wave 200 originates in the high-impedance material and transitions to the low-acoustic impedance target medium causing the acoustic wave 200 “focus” or converge to the target tissue.

In one embodiment a transducer 150 has a convex emitting surface 310 (or has a lens 302 defining a convex surface 310), according to at least one embodiment of the present disclosure. The lens 302 may be acoustically coupled to the transducer 150 or may be formed integrally therewith. In one embodiment, the lens 302 is made of a material with higher acoustic impedance than the target medium (water/tissue). Accordingly, an acoustic wave 312 originates in the high-impedance material and transitions to the low-acoustic impedance target medium causing the acoustic wave 312 to “defocus” or diverge to the target tissue. In one embodiment, the lens 302 is made of a material with higher acoustic impedance than the target medium (water/tissue). Accordingly, an acoustic wave 312 originates in the high-impedance material and transitions to the low-acoustic impedance target medium causing the acoustic wave 312 to “defocus” or diverge to the target tissue.

In various embodiments, for example, the transducer elements 150a-150h can be activated in a predetermined sequence to selectively generate convergent/divergent/planar acoustic waves, such as, for example, a convergent acoustic wave 314, or a divergent acoustic wave 312. To generate a converging acoustic wave 314, for example, the outer transducer elements 150a, 150h are initially energized and after a time delay the adjacent inner transducer elements 150b, 150g are energized. In various embodiments, a time delay is in a range of 0.1 μs to 10 s, including 0.1 μs, 0.2 μs, 0.3 μs, 0.4 μs, 0.5 μs, 1 μs, 5 μs, 10 μs, 15 μs, 20 μs, 25 μs, 30 μs, 35 μs, 40 μs, 50 μs, 60 μs, 70 μs, 80 μs, 90 μs, 0.1 ms, 0.2 ms, 0.3 ms, 0.4 ms, 0.5 ms, 1 ms, 5 ms, 10 ms, 50 ms, 100 ms, 500 ms, 1 s, 2 s, 5 s, and 10 s and any values and ranges therein. The next adjacent inner transducer elements 150c, 150f are energized after a second time delay. Finally, the inner transducer elements 150d, 150e are energized after a third time delay. This pattern can be repeated to generate the converging acoustic wave 314. The first, second, and third time delays may be equal or may vary in order to generate more complex acoustic waves. Alternatively, the transducer elements 150a-150h may be energized in reverse order to produce a diverging acoustic wave using equal or different time delays. The transducer elements 150a-150h can be interchangeably configured to transmit or receive acoustic waves.

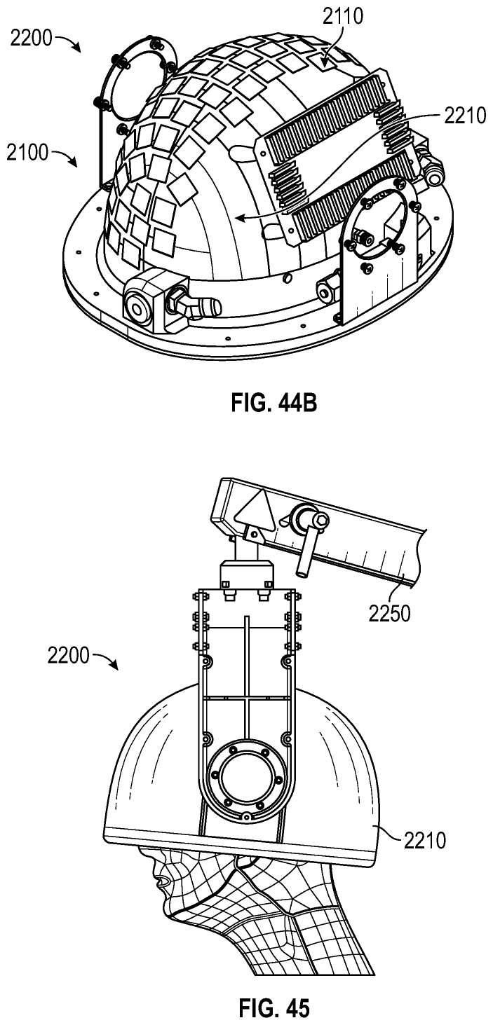

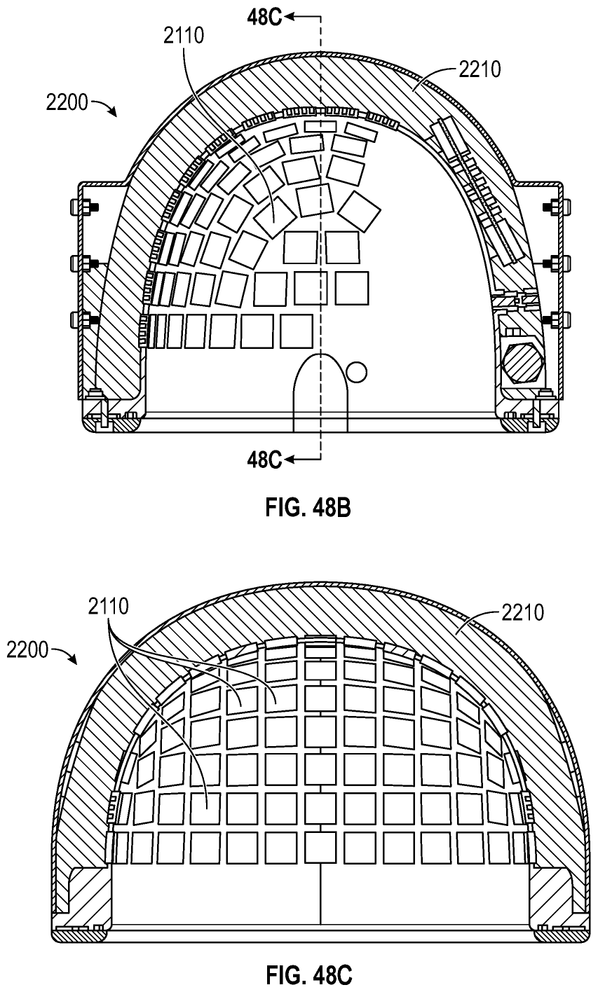

In one embodiment, an array of ultrasound transducers 150 includes an internal element 420 surrounded by concentric rings 410, according to at least one embodiment of the present disclosure. In one embodiment, an internal element 420 is surrounded by concentric elements 410. The concentric arrangement can also include, for example, curved or arcs that share one or more common centers, such as in FIGS. 44A and 44B and FIGS. 48A-C, wherein the general curved or arc includes flat, planar components (e.g., elements or groups of elements, arrays, sub-arrays, etc.). Each transducer 150 can be adapted and configured to produce an acoustic wave with variable focus. One way to accomplish this can be with each transducer 400 having concentric rings 410 (e.g., an annular array). Each concentric element 410 can be driven with a different signal. In one embodiment to focus the acoustic wave, the signal going to the inner element 420 may be progressively more delayed than the outer of the concentric ring 410. The acoustic waves from each concentric ring 410 may converge at a point. In one embodiment to defocus the acoustic wave coming from an annular array, the acoustic wave at the outer of the concentric rings 410 may be progressively more delayed relative to the inner element 420. One way to make an embodiment of an annular array can be with concentric rings 410 of equal area. In another embodiment, the annular array may comprise concentric rings 410 of unequal area.

In one embodiment, a transducer 150 comprising internal elements 452 is arranged in 2-dimensional (2D) grid array 450, according to at least one embodiment of the present disclosure. In one embodiment, one or more elements 452, 454 in the 2-dimensional (2D) grid array 450 is a flat, planar emitting surface that produce a planar acoustic wave. Each internal element 452 of the 2D grid transducer array 450 can be driven with a different signal. In one embodiment, to produce a converging acoustic wave (e.g., “focus”) from the dimensional (2D) grid array 450, the signal applied to the inner element 454 may be progressively more delayed than the signal applied to the outer elements of the 2D grid transducer array 450. In one embodiment to produce a diverging acoustic wave (e.g., “defocus”) from the dimensional (2D) grid array 450, the acoustic wave produced by the outer elements 454 may be progressively more delayed relative to the inner element 452. In one embodiment to produce a steered beam, elements are delayed in a standard delay pattern such that the acoustic beams converge at the desired location. In one embodiment, each of the internal elements 452 and/or external elements 454 of the 2D grid transducer array 450 may define an equal area. In another embodiment, each of the internal elements 452 and/or external elements 454 of the 2D grid transducer 450 array may define an unequal area.

In one embodiment, the transducer 150, 400, 450 may be implemented as a single transducer comprising multiple piezoelectric elements with acoustically/electrically independent sections arranged in an array, In other embodiments, the transducer 150, 400, 450 may be implemented as different transducers working in a coordinated manner. In one embodiment, there is little or no distinction from a physics perspective between a single transducer with multiple elements and different transducers working in coordination. In one embodiment, there are several and/or significant distinctions from a physics perspective between a single transducer with multiple elements and different transducers working in coordination. The elements of an array can be sized on the order of a wavelength. In various embodiments, the wavelength is 0.1 mm-5 mm, 0.1 mm-4 mm, 0.1 mm-3 mm, 0.1 mm-2 mm, 0.1-1.5 mm, 0.1 mm-1 mm, 0.5 mm-3 mm, 0.5 mm-2 mm, 0.5 mm-1.5 mm, 0.5 mm-1 mm, 1 mm-5 mm, 1 mm-4 mm, 1 mm-3 mm, 1 mm-2 mm, 1 mm-1.5 mm, 1.5 mm-4 mm, 1.5 mm-3 mm, 1.5 mm-2 mm, 2 mm-5 mm, 2 mm-4 mm, 2 mm-3 mm, 3 mm-5 mm, 3 mm-4 mm, 0.1 mm-10 mm, and values therein. In one embodiment, the transducer 150, 400, 450 may be implemented as a single transducer comprising a plurality of elements implemented as an annular array or as a grid array. In another embodiment, the transducer 150, 400, 450 may be implemented as a plurality of individual transducers.

In one embodiment, each of the transducers 150, 400, 450, or elements thereof, are non-invasive and may be implemented in a suitable size and shape to fit on the body part of the patient. Also, the individual number and arrangement of transducer elements may be selected to fit on the body part of the patient. In one embodiment, an array of flat, planar emitting surfaces is arranged with a curvature configured to direct each flat element in the array to emit the planar acoustic wave normal to a body surface, such as a skull. In one embodiment, an array of flat elements may be arranged along a surface configured specifically to position one or more of the individual flat elements normal or perpendicular to a body surface in order to emit each individual planar acoustic wave normal or perpendicular to that body surface. For example, this arrangement to align the flat, planar acoustic waves emitted from the flat elements may be arranged to surround a body structure, such as a skull, with a radius of curvature in a range of 50 mm to 200 mm, including 50 mm to 175 mm, 50 mm to 150 mm, 50 mm to 125 mm, 50 mm to 100 mm, 60 mm to 150 mm, 60 mm to 140 mm, 70 mm to 130 mm, 70 mm to 110 mm, 75 mm to 150 mm, 75 mm to 125 mm, 75 mm to 100 mm, 80 mm to 120 mm, 80 mm to 100 mm, 90 mm to 130 mm, 90 mm to 110 mm, 100 mm to 125 mm, 100 mm to 150 mm, 100 mm to 175 mm, 125 mm to 150 mm, 125 mm to 175 mm, 125 mm to 200 mm, 150 to 175 mm, 150 mm to 200 mm, 125 mm, 150 mm, 165 mm, 175 mm, 200 mm, and values therein. In various embodiments, the arrangement may have a single radius of curvature, as in a portion of a sphere. In various embodiments, this arrangement may have two or more radii of curvature, such as a primary curvature across an anterior/posterior axis of a skull, and a secondary curvature across a lateral axis of the skull. In one embodiment, the transducer 150, 400, 450, or elements thereof, may be made of piezoelectric and/or single crystal material which converts electrical energy to ultrasonic energy. The transducer 150, 400, 450 also can receive back ultrasonic energy and convert it to electrical energy for monitoring and/or imaging. Each of the transducers 150, 400, 450, or elements thereof, may be adaptively configured to produce acoustic waves by collaborative transducer performance. For example, each of the transducers 150, 400, 450, or elements thereof, may be selectively controlled to operate either as a transmitter or as a receiver by a controller as described herein. Further, each of the transducers 150 400, 450, or elements thereof, may be selectively energized and actuated to produce convergent, divergent, or planar acoustic waves as discussed in more detail in the present description.

In one embodiment, the acoustic wave produced by the transducer 150, 400, 450 may be characterized by phase and/or delay. The phase and/or delay may be employed to measure a relative shift in time between two acoustic waves. The phase is the amount of time shifted between two acoustic waves relative to the period of the two acoustic waves (e.g., measured in degrees or radians). The delay is a measure of the amount of time shifted between two acoustic waves (e.g., measured in milliseconds). In various embodiments, a time delay is in a range of 0.1 μs to 10 s, including 0.1 μs, 0.2 μs, 0.3 μs, 0.4 μs, 0.5 μs, 1 μs, 5 μs, 10 μs, 15 μs, 20 μs, 25 μs, 30 μs, 35 μs, 45 μs, 50 μs, 60 μs, 70 μs, 80 μs, 90 μs, 0.1 ms to 10 s, including 0.1 ms, 0.2 ms, 0.3 ms, 0.4 ms, 0.5 ms, 1 ms, 5 ms, 10 ms, 50 ms, 100 ms, 500 ms, 1 s, 2 s, 5 s, and 10 s and any values and ranges therein. Delay and phase are often used interchangeably. For example, although “delay” may be described in units of degrees or radians, it is well understood that in certain embodiments, “delay” is an abbreviation for “phase delay.” In various embodiments, the phase delay is 0.2, 0.4, 0.5, 0.6, 0.8, 1.0, 1.2, 1.4, 1.5, 1.6, 1.8, 2.0, 2.2, 2.4, 2.5, 2.6, 2.8, 3.0, 3.3, 3.5, 3.8, 4.0, 4.5, 5.0, 5.5, 6.0, 6.28 radians and values and ranges therein. In various embodiments, the phase delay is 10, 20, 40, 45, 50, 60, 80, 90, 100, 120, 125, 130, 135, 140, 150, 160, 170, 180,190, 200, 220, 225, 230, 240, 250, 260, 270, 280, 300, 310, 320, 340, 350, 360 degrees and values and ranges therein, such as 0-300, 0-270, 0-240, 0-180, 0-120, 0-90, 45-225, 90-300 degrees. For a single acoustic wave pulse, the delay between the peaks of two acoustic wave pulses can be expressed in terms of time because a phase shift is associated with a periodic signal. For repeating acoustic waves, the relative delay is often measured terms of phase. For continuous, periodic acoustic waves, delaying an integer number of periods should have no effect because, by definition, a periodic signal exhibits symmetry over full period shifts. For pulses of a repeating acoustic wave (e.g., 1000 cycles of a sine wave), the acoustic wave can be delayed by an integer number of cycles. The beginning and end of the wave packet will have some edge effect when one signal begins/ends before the other. In the middle of the two wave packets, there will be no effect (provided the signals still overlap).

In various embodiments, the transducers 150, 400, 450 may be driven at ultrasonic frequencies in a range of about 20 kHz to about 12 MHz, including, for example, 20 kHz, 50 kHz, 100 kHz, 250 kHz, 400 kHz, 500 kHz, 550 kHz, 600 kHz, 650 kHz, 700 kHz, 750 kHz, 800 kHz, 850 kHz, 900 kHz, 950 kHz, 1 MHz, 1.05 MHz, 1.1 MHz, 1.15 MHz, 1.2 MHz, 1.25 MHz, 1.3 MHz, 1.35 MHz, 1.4 MHz, 1.45 MHz, 1.5 MHz, 2 MHz, 3 MHz, 4 MHz, 5 MHz, 10 MHz, 12 MHz, 20 MHz, 25 MHz, 50 MHz, 75 MHz, 100 MHz, and any values and ranges therein, such 0.5 to 1.5 MHz, 0.6 to 1.4 MHz, 0.7 to 1.1 MHz, 0.8 to 1.2 MHz, 1 to 5 MHz, etc. More particularly, the transducers 150, 400, 450 may be driven at ultrasonic frequencies in a range of about 650 kHz to about 2.00 MHz. In various embodiments, the transducers 150, 400, 450 may be driven at ultrasonic frequencies in a range of 600-1200 kHz, 700-1050 kHz, 700-1100 kHz, 700 kHz to 1.2 MHz, 900 kHz to 1.20 MHz, 975 kHz-1.15 MHz, and as examples, in one embodiment, at 1 MHz, 1.03 MHz, 1.06 MHz, 1.10 MHz, 1.20 MHz, etc.). In one embodiment, an ultrasound transducer system 100 drives a transducer array with signal driving patterns with a frequency swept continuously or step-wise between 600 kHz, 700 kHz, 750 kHz, 900 kHz, 950 KHz, 1 MHz and 1.05 MHz, 1.1 MHz, 1.15 MHz, 1.2 MHz. In various embodiments, a frequency is swept with 2, 4, 8, 16, 32, 64, 128, 256, 512, 1024 or more phase randomizations for each element in the array. In one embodiment, each phase randomization is unique for each element in the array.

In various embodiments, the transducers 150, 400, 450 may be driven with a signal from the controller 130 to have the transducer(s) 150 deliver therapeutic treatments with a normalized, randomized, and/or incoherent acoustic pressure field. In various embodiments, a unique drive pattern is provided to each element in the array, where phase and frequency of the drive signal is varied for each pulse. During each discrete pulse, the drive signal waveform phase of the individual elements is randomized to reduce or prevent the formation of pressure or thermal focal points within the treated tissue. A unique element waveform phase pattern is randomly generated with each new pulse sequence so that the pattern changes from pulse to pulse. In one embodiment, the drive frequency of the individual elements in the array is varied across a range of 500 kHz-2.0 MHz (e.g., 500-1200 kHz, 600-1200 kHz, 700-1200 kHz, 800-1100 kHz, 600-1100 kHz, 700-1100 kHz, and ranges and values therein) within each pulse and/or between discrete pulses. In one embodiment, a unique drive signal to each element for the duration of a single pulse, and is then alternated to a new unique combination for the subsequent pulse. In one embodiment, the phase is randomized across all elements in the array. In one embodiment, the phase is randomized across all elements in the array, which constitutes unique drive patterns for each element in the array for the single pulse. In one embodiment, the next subsequent pulse broadcasts a new frequency, and again randomization is re-applied across all elements in the array.

In various embodiments, the ultrasound transducer system 100 emits an Absolute Peak Pressure (APP) representing the maximum absolute instantaneous pressure. In various embodiments, the transducers 150, 400, 450 create a normalized, randomized, and/or incoherent acoustic pressure field with an Absolute Peak Pressure (APP) of between 1 MPa-10 MPa (e.g., 1-5 MPa, 5-10 MPa, 2-7 MPa, 3-5 MPa, 7-9 MPa, 1, 1.5, 2, 2.5, 3, 3.3, 3.5, 3.7, 4, 4.4, 4.5, 4.7, 5, 5.5, 6, 6.5, 7, 7.5, 8, 8.5, 9, 9.5, and 10 MPa, including ranges and values therein).

In various embodiments, the ultrasound transducer system 100 emits a Pulse Average Pressure (PAP) representing the average absolute instantaneous pressure between the time when the time integral of the pulse intensity integral reaches 10% and 90% of its final value for each burst, and is then averaged across all bursts. In various embodiments, the transducers 150, 400, 450 create a normalized, randomized, and/or incoherent acoustic pressure field with an Pulse Average Pressure (PAP) of between 0.1 MPa-5 MPa (e.g., 0.1-3 MPa, 0.5-3 MPa, 1-2 MPa, 3-5 MPa, 0.1, 0.5, 1, 1.3, 1.5, 1.8, 2, 2.3, 2.5, 2.7, 3, 3.3, 3.5. 3.6, 4, 4.4, 4.5, 4.7, and 5 MPa, including ranges and values therein).

In various embodiments, a normalized pressure field comprises a ratio of Absolute Peak Pressure (APP): Pulse Average Pressure (PAP) of 1.01, 1.1, 1.25, 1.4, 1.5, 1.6, 17. 1.8, 2.0, 2.2, 2.3, 2.4, 2.5, 2.6, 2.7, 2.8, 2.9, 3.0, 3.5, 4.9, 4.5, 5.5, 6.0 to 1 (e.g., 1.01-6.0, 1.0-5.0, 2.0-3.0 to 1 and ranges and values therein). In various embodiments, a normalized pressure field comprises an Absolute Peak Pressure (APP) that is 101%-400% of an Pulse Average Pressure (PAP), e.g., 101%, 110%, 125%, 140%, 150%, 160%, 175%, 190%, 200%, 250%, 300%, 400%, 101%-200%, 125%-175%, 140%-190%, including ranges and values therein).

Returning to FIG. 3C, this illustrated embodiment is a partial cutaway view of a transcranial sonodynamic therapy device placed over the head of a patient showing a partial view of the skull 510 and brain of the patient and multiple transducers 150 with one transducer emitting energy into the brain of the patient, according to at least one embodiment of the present disclosure. It can be possible to take measurements or get a rough image of the skull 510 as shown in FIG. 3C. This can be facilitated if the transducers 150 are fixed to a rigid shell and their relative positions and orientations are known. Rough measurements can be used to adjust the treatment algorithm by measured parameters such as skull thickness, “t” or skull density, “ρ.” Each transducer 150 may send out an acoustic pulse and listen for an echo. The echoes can be used for a quick estimate of the skull thickness, “t,” or skull density, “ρ,” under each transducer 150. For treatment of tumors in other body parts of the patient, the sonodynamic therapy device may be adapted and configured to the couple to the body of the patient.

For designs with transducers 150 that have an adjustable focus, the focus of each transducer 150 can be set beforehand with treatment planning. Alternatively, the transducers 150 can adjust their focus automatically based on temperature readings with a temperature monitor 810 of the body, such as at the head or based on skull thickness, “t,” measurements. In one embodiment, temperature readings are used as feedback for increasing or decreasing intensity to stay within a safe thermal dose ranges for tissue, such as a temperature below 45° C. such as 44° C. 43° C. 42° C., 41° C., 40° C., 39° C. 38° C., 37° C. 36° C., or 35° C., and any range or value therein.

The amplitude of the electrical drive signal driving the transducers 150 can be controlled or modulated. In some cases, it can be beneficial to modulate the electrical drive signal driving the transducers 150 based on the temperature of the head or other body part being treated. For example, if the temperature sensors 810 are detecting a sharp rise in temperature, the amplitude of the transducers 150 can be decreased, shut off for a period, or the duty cycle can be decreased. By modulating the intensity of the acoustic pulses, the temporal average acoustic intensity may be regulated to activate the sensitizer while maintaining the temperature of the tissue cells below a target temperature (e.g., below 45° C., 44° C., 43° C., or 42° C., such as 41° C., 40° C., 39° C., 38° C., 37° C., 36° C., or 35° C., and any range or value therein) capable of causing thermal damage to the cell and in some circumstances necrotic cell death in some embodiments. By modulating the intensity of the acoustic pulses, the pulse average acoustic intensity may be regulated to activate the sensitizer while maintaining the temperature of the tissue cells below a target temperature (e.g., below 45° C., 44° C., 43° C., or 42° C., such as 41° C., 40° C., 39° C., 38° C., 37° C., 36° C., or 35° C., and any range or value therein) capable of causing thermal damage to the cell and in some circumstances necrotic cell death in some embodiments. In another embodiment, sonodynamic therapy can function at a variety of different frequencies. Each frequency can transmit through a tissue efficiently with certain thicknesses of tissues. Using a variety of frequencies can allow a non-invasive sonodynamic therapy device 100 to operate on a broad range of tissue thicknesses, “t.” Each frequency can transmit through a skull 510 efficiently with certain thicknesses of skulls. Using a variety of frequencies can allow a non-invasive sonodynamic therapy device 100 to operate on a broad range of skull thicknesses, “t.”

In embodiments where the transducers 150 can operate at multiple frequencies, the frequency of each transducer 150 can be selected manually by an operator or automatically. As stated in the foregoing description, the transducers 150 may be driven at ultrasonic frequencies in a range of about 20.00 kHz to about 12.00 MHz, including, for example, 20 kHz, 50 kHz, 100 kHz, 250 kHz, 500 kHz, 1 MHz, 1.1 MHz, 1.2 MHz, 1.3 MHz, 1.4 MHz, 1.5 MHz, 2 MHz, 3 MHz, 4 MHz, 5 MHz, 10 MHz, 12 MHz, and any values and ranges therein. More particularly, the transducers 150 may be driven at ultrasonic frequencies in a range of about 650.00 kHz to about 2.00 MHz. In one embodiment, the transducers 150 may be driven at ultrasonic frequencies in a range of about 900.00 kHz to about 1.20 MHz and (in some embodiments) at about 1 MHz, 1.03 MHz, 1.06 MHz, 1.10 MHz, 1.20 MHz, etc. The frequencies can be preselected by a physician. The frequencies can be selected based on a measurement of head anatomy (e.g., skull thickness, “t” or skull density, “ρ”). For example, each transducer 150 can send out a sequence of pulses to measure the thickness of the skull 510 closest to it. Based on the result of the skull thickness, “t,” or skull density, “ρ,” measurement, an algorithm can be used to select frequencies from a set of frequencies or from a range of frequencies that may be best suited for the skull thickness, “t,” or skull density, “ρ,” and energize the transducers 150 accordingly.

The size and shape of the transducers 150, as can be seen in FIG. 3B, may vary across various disclosed embodiments. For a cost-effective and simple system, larger transducers 150, which may have directional acoustic waves, and which may have more directional acoustic waves, may be used. Large transducers 150 can be made less directional by applying to each transducer 150 an acoustic lens that bends the acoustic waves as described further elsewhere herein. For a system that can conform to the skull, smaller transducers 150, which can radiate more broadly than larger transducers 150, can be used. Such small transducers 150 can have a greater ability to image or beam steer as an array.

In some embodiments, the acoustic wave 200 is focused to a small region (e.g., as shown in FIG. 4A), such as a point, sphere, oval, circular etc. region (e.g., 0.1-1 mm³, 0.5-2 mm³, 0.75-2.5 mm³, 3-5 mm³, 2-6 mm³, 1 mm³, 2 mm³, 3 mm³, 4 mm³, 5 mm³, 6 mm³, 7 mm³, 8 mm³ and values and ranges therein), Instead of focusing an acoustic wave 200 to a small point, in some embodiments, the acoustic wave 200 can be defocused to minimize the spatial variation of the acoustic wave intensity in a body part, such as the brain (e.g., over the volume of the entire brain, a portion of the brain, 100 mm³-10,000 mm³, 2000 mm³-6000 mm³, 4000 mm³-8000 mm³, 10,000 mm³, 9000 mm³, 8000 mm³, 7000 mm³, 6000 mm³, 5000 mm³, 6000 mm³, 3000 mm³, 2000 mm³, 1000 mm³, 500 mm³, 250 mm³, 100 mm³, or more) as shown in FIG. 4A. The size and shape of the transducers 150 may defocus or focus each transducer 150. Defocused transducers can be formed using a transducer 150 with a convex emitting surface. In one embodiment, design of the transducers can focus the sound from each transducer 150 using a concave emitting surface with a center of curvature where the sound can focus. As shown in FIG. 4A, an array of transducers 150a-150h can be used to generate acoustic waves that are convergent, divergent, or more complex. In various embodiments, the array has a dimension (length, width, diameter, etc.) In various embodiments, the transducers 150a-150h and/or elements 452, have a dimension (e.g., length, radius, diameter) in the range of 0.5 mm to 20 mm, including 0.5 mm, 1 mm, 3 mm, 5 mm, 7 mm, 10 mm, 12 mm, 15 mm, 18 mm, and 20 mm including any values and ranges therein. In various embodiments, the transducers 150a-150h and/or elements 452, have a dimension (e.g., length, radius, diameter) in the range of 5 mm to 150 mm, including 5 mm, 10 mm, 30 mm, 50 mm, 70 mm, 100 mm, 120 mm, 130 mm, 140 mm, 150 mm, 160 mm, 170 mm, 180 mm, 190 mm, and 200 mm including any values and ranges therein. In some embodiments, the diameter at the exit plane of the transducer is at least 10-50% (e.g., 10, 20, 30, 40, 50% and values and ranges therein) larger than the radius of curvature.

Each transducer 150 can cycle through several frequencies so that at least one of the frequencies can transmit nearly optimally for the given skull thickness, “t” or skull density, “ρ.” Each transducer 150 may also sweep continuously from one frequency to another. A frequency can be pre-selected for each transducer 150 based on the thickness of skull 510 nearest to it (e.g., during treatment planning by the physician).

Each transducer 150 can transmit test signals and monitor the reflected sound with a reflection monitor 820 (also referred to as a reflected acoustic energy monitoring device). In various embodiments, a reflection monitor 820 (or a reflected acoustic energy monitoring device 820) is configured to modulate the average acoustic pressure across the volumetric field upon detection of a degree of reflected acoustic energy. In various embodiments, a reflection monitor 820 is configured to measure acoustic reflections so the controller can automatically determine which frequency or frequencies can work best for that one of the transducers 150. The test signals can be used to measure the tissue (e.g., skull) thickness, “t,” or tissue density, “ρ,” directly by measuring delays in pulse echoes, or they can be used to detect the relative amount of reflected acoustic energy. In one embodiment, a reflection monitor 820 measures a distance between the ultrasound system (e.g., patient interface, transducer, housing, etc.) and a portion of the body of the patient. In one embodiment, a reflection monitor 820 is a standoff sensors for measure distance from a target tissue (e.g., skin surface, skull, brain, organ, etc.). In one embodiment a standoff sensor can adjust coolant flow rate and/or pressure to adjust a flexible, elastic membrane of the patient interface/cooling system to adjust a treatment distance via inflation/deflation of the membrane.

Each transducer 150 can be made up of a broad-spectrum ultrasonic transducer or can be made up of several smaller transducers (e.g., piezo-electric elements as shown in FIGS. 4A-C) designed to work at particular frequencies. Each transducer 150 can have an element specifically designed to monitor the waves reflected from the patient's body. In the case where the transducers 150 are made of several smaller transducers 150, while one transducer 150 is transmitting sound, the other transducers 150 may be used to transmit and/or monitor the incoming acoustic pulses.

Of all the frequencies that work with sonodynamic therapy, a subset of frequencies can be selected to best cover a range of common tissue thicknesses, “t.” Frequencies that share many common factors (e.g., harmonics such a 1 MHz and 2 MHz) may not make good choices to cover the greatest number of skull thicknesses because many of the transmission peaks between the two frequencies can be shared. Frequencies without many or any common factors (e.g., coprime numbers) may make for good choices for frequencies because the transmission peaks can occur at different skull thicknesses. In one embodiment, a sensor detects ultrasound signals and the system reviews a signal spectrum to identify harmonics, subharmonics, and/or ultraharmonics to alter the ultrasound signal frequency, intensity, or other parameter.

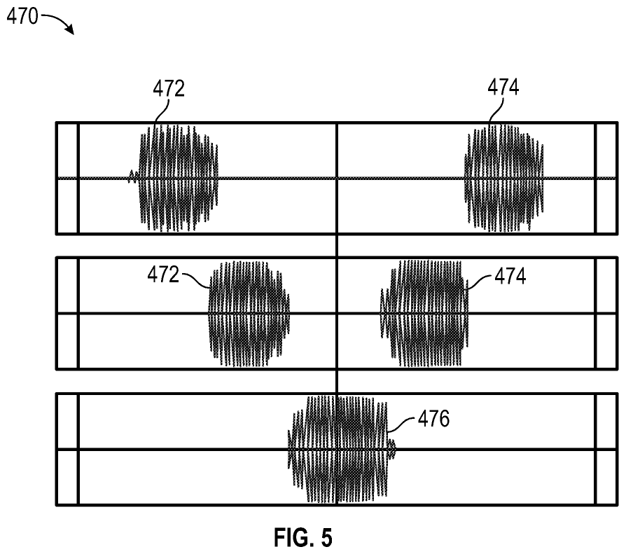

FIG. 5 is a diagram 470 of two acoustic ultrasonic pulses 472, 474 without delay that constructively interfere, according to at least one embodiment of the present disclosure. In one embodiment, the transducers 150, 400, 450 may be adapted and configured to produce a “focused”, “defocused”, or planar acoustic wave by coordinating time between multiple acoustic wavefronts and producing wavefronts that constructively interfere. The coordination of acoustic wavefronts is independent of the vergence of the acoustic wavefronts. The point at which the wavefronts focus can be adjusted by delaying one signal relative to another. The diagram 470 shown in FIG. 5 shows two pulses 472, 474 produced without any relative delay. The two pulses 472, 474 constructively interfere when they reach the center and may be said to be focused or defocused in the center to produce a combined pulse 474, 476. If the acoustic pulse 472 on the left is delayed relative to the acoustic pulse 474 on the right, the two pulses 472, 474 would meet at a point left of center, thus shifting the point of constructive interference to the left of center. Likewise, if the acoustic pulse 474 on the right is delayed relative to the acoustic pulse 474 on the right, the two pulses 472, 474 would meet at a point to the right of center, thus shifting the point of constructive interference to the right of center. In another embodiment, a mixture of convergent/divergent/planar acoustic waves may be timed to meet and constructively interfere at one location. A divergent acoustic wave may be timed to meet and destructively interfere at one location. In other embodiments, a mixture of convergent/divergent/planar acoustic waves may be timed to meet and constructively interfere at one, two, three, five, ten, or more locations. Control of the converging and diverging wavefronts produced by the transducers 150, 400, 450 can be taken into account as part of pretreatment planning. In one embodiment, based on inputs from the pretreatment planning processes the controller can adaptively modulate the transducers 150, 400, 450 such that the acoustic wavefronts coordinate to preferentially target a desired treatment region. In one embodiment a digital imaging and communications (DICOM) image from a camera, computerized tomography (CT), magnetic resonance (MRI) or other imaging source could be an input to the device controller to generate customized modulation pattern that optimizes the treatment region for a particular patient. In one embodiment, a DICOM image is used to determine characteristics of a tissue for treatment and surrounding anatomy, such as a skull thickness, and/or use image processing to interpret an average skull thickness of a patient and any one or more of intensity, amplitude, and frequency of the treatment is calibrated based on the tissue characteristic (e.g., skull thickness and/or average skull thickness). In one embodiment, a tissue characteristic, such as average skull thickness, of a patient is used to calibrate a treatment intensity. In one embodiment, a tissue characteristic, such as average skull thickness, of a patient is used to calibrate a treatment amplitude. In one embodiment, a tissue characteristic, such as average skull thickness, of a patient is used to calibrate a treatment frequency. In one embodiment, a DICOM image is used to determine a tissue characteristic, such as skull density, and/or use image processing to interpret an average skull density of a patient and any one or more of intensity, amplitude, and frequency of the treatment is calibrated based on the skull density and/or average skull density. In one embodiment, a tissue characteristic such as average skull density of a patient is used to calibrate a treatment intensity. In one embodiment, a tissue characteristic, such as average skull density of a patient is used to calibrate a treatment amplitude. In one embodiment, a tissue characteristic, such as average skull density of a patient is used to calibrate a treatment frequency. In another embodiment the pretreatment planning could include selection of a transducer type or arrangement of transducer types that will produce an optimized treatment region for a particular disease state. In another embodiment, the patient interface may come in various arrangements that can be selected during pretreatment planning to coordinate the transducer(s) in an arrangement for treatment. In one embodiment, volumetric imaging data is acquired to plan the targeting of a sonodynamic treatment. In one embodiment, “defocused” acoustic waves may be measured based on the volume of tissue treated according to the number of nodes and antinodes. A histogram of intensities or pressures over some volume may be employed to measure “defocused” acoustic waves. In one embodiment, a dose-volume histogram may be employed in planning sonodynamic therapy. Alternatively, a cumulative histogram may be employed.

Energy lost as the sound passes through the skull may be converted into heat primarily in the skull. The temperature of the skull can begin to heat up and, over time, heat can disperse to nearby tissue. Most of the heating can originate at the outer surface of the skull and disperse into the skin and other layers of bone. Above certain intensities, the blood might be unable to transport enough heat away, and the temperature in the bone and skin can rise to unsafe or uncomfortable levels. Adding more transducers into the system can decrease the intensity at which this threshold can be reached because the blood can be warmed by each successive transducer it passes and lose its ability to absorb additional heat from the tissue. There can be several ways to combat the effects of heating. In particular, cooling, intermittent treatment, monitoring, and transducer modulation can be used to reduce the consequences of heating.

In various embodiments, a cooling system 600 may be implemented to keep the temperature of the patient interface and surrounding tissue within safe, comfortable levels. A cooling layer (e.g., of a coolant or cooling fluid, such as water) may be provided between the transducers 150 and the patient. The cooling layer can be made of a flexible membrane or balloon that can conform to the patient's anatomy. In one embodiment, a cooling layer may be reusable and, thus, may involve cleaning between each use.

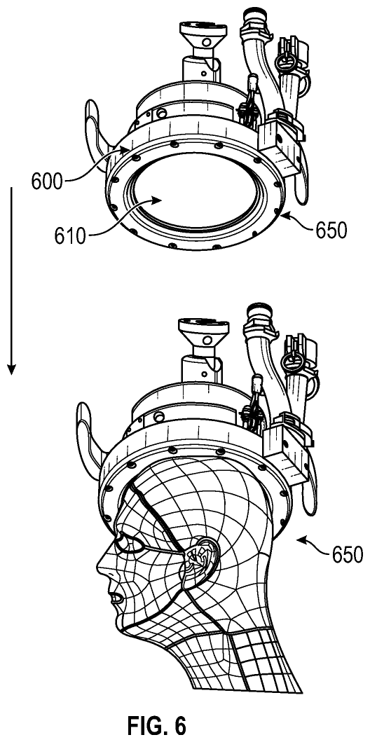

In one embodiment shown at FIG. 6, a cooling system 600 can be made of a flexible cavity with an inlet and an outlet for a coolant such as water to circulate. In various embodiments, the coolant is a fluid, liquid, gel, gas (e.g., such as water) can flow at a rate (liters per minute “LPM”) in a range of 1-50 LPM (e.g., 1-10 LPM, 10-40 LPM, 20-40 LPM, 25-50 LPM, 30-40 LPM, and any ranges therein) with a rate of 1, 2, 5, 10, 15, 20, 25, 30, 35, 40, 45, 50 LPM, and any ranges and values therein). The patient anatomy, such as a head of the patient, can be inserted into or against a concave shape (e.g., a “bowl”) with an elastic interface. The elastic interface (e.g., a flexible patient interface, membrane, etc.) can couple to a portion of the patient for treatment. In various embodiments, the elastic interface may change in size and shape depending on the flow rate or pressure of the coolant, which can be controlled via changes in the coolant flow rate and/or adjustment of coolant pressure. In one embodiment, the cooling system 600 comprises one, two, three or more pumps. In one embodiment, the pump(s) are modulated based on a coolant pressure set point in the elastic interface. The coolant can fill up the space between the patient's head and the bowl.

Similar to the single cavity design, the cooling system 600 can circulate the coolant with one or more inlets, outlets, and pumps at various rates, such as 1, 5, 10, 15, 20, 25, or 30 liters per minute (LPM), and any values and ranges therein to keep the temperature of the coolant, the system, and/or the patient from rising significantly (e.g., limiting temperature increases to 10, 5, 4, 3, 2, 1° C. or less. In one embodiment, coolant in the cooling system 600 also couples the ultrasound transducers 150 to the patient.

In one embodiment, the cooling system 600 includes a patient interface with cooling channels distributed throughout. The cap 160 can have one long loop of cooling channels, or it can have several independent loops. A system with several cooling loops can be connected to a single inlet and outlet tube via a manifold, or they can be controlled independently. Water or other heat transfer fluid can be circulated through the cooling channels to exchange heat generated either by the transducers 150, the patient's body, or a combination thereof. In one embodiment, a thin film compliable membrane 610 that is installed across the face of the transducer that is flexible, elastic, and configured to conform to a shape of the body of the patient for treatment. In one embodiment, a thin film compliable membrane 610 forms a fluid-filled pocket configured for thermal and/or acoustic coupling to the portion of the body of the patient. In one embodiment, the cooling system 600 comprises the membrane 610 to provide active cooling to the portion of the body of the patient for treatment.

In one embodiment, the coupling membrane 610 is configured to trap a degassed circulating fluid (e.g., water, saline, cooling fluid, acoustic coupling material, gel) between the ultrasound array and the patient. The degassed circulating fluid provides an acoustical coupling pathway from the individual ultrasound elements to the membrane 610 face. The circulating fluid also provides active cooling that mitigates the potential for residual heat buildup at the patient entry plane and/or the ultrasonic elements. A manifold with a plurality of nozzles may be incorporated into the membrane assembly to further direct circulating fluid towards the wet face of the coupling membrane to increase cooling capacity directly at the patient interface. In one embodiment, the coupling membrane 610 with fluid backing provides a conformable interface that adapts and molds to the shape of the local anatomy at the treatment site. In one embodiment, ultrasonic coupling gel is additionally placed at the treatment site as part of the interface between the patient and coupling membrane. The conformable fluid filled membrane 610, along with ultrasonic coupling gel ensures good acoustical coupling between the transducers and patient. The conformable fluid filled membrane 610, along with ultrasonic coupling gel ensures good thermal coupling between the transducers and patient. In one embodiment, the coupling membrane 610 is the only portion of the sonodynamic treatment device that has direct patient contact. The membrane 610 is made from a well characterized elastomer with a known biocompatibility profile for patient contact. In one embodiment, the coupling membrane 610 can be removed and replaced as needed between patient uses. In one embodiment, the coupling membrane 610 is attached to a de-couplable bezel or housing that can be removably attached (e.g., with one or more interfaces, locking features, latches, threads, etc.) from the ultrasound array. In various embodiments, the fluid (e.g., water) can flow past all regions of the body (e.g., head, torso, etc.) that can absorb heat. The fluid can be pumped to keep the fluid temperature from rising which would decrease the cooling efficacy of the fluid. Like patches with multiple transducers 150, each patch may have its own cooling channels. The cooling channels can be fluid-filled tubes that may be larger and heavier than the wires going to the transducers 150. The number of unique cooling channels can be optimized to avoid excessive weight in the cooling layer.

The effect of heating can be readily monitored with one or more temperature sensors 810 and reduced with the fluid cooling system 600. A layer of cool, degassed water between the ultrasonic transducers 150 and the head can serve a dual function of coupling the head to the transducers 150 and controlling the temperature of the patient's body and/or tissue. In one embodiment, prior to ensonification, a portion of the patient's body can be cooled for several minutes by a flow of coolant. Once the treatment begins, the temperature of the patient's body can be monitored continuously with one or more temperature monitors 810, which can modulate the treatment over the entire portion of the patient's body, or it can individually modulate each transducer 150. Even without continuous monitoring of the patient's body temperature, a safe treatment algorithm can be devised with intermittent treatment and continuous cooling with a margin of safety for the patient. Intermittent treatment can also be more effective than the same effective treatment time done continuously due to the rate limiting step of oxygen diffusion around the sonosensitizer. In one embodiment, surface temperature monitoring is performed with one or more temperature monitors 810. In any case, it can be possible to monitor the temperature throughout the skull using a variety of thermometry of deep-seated tissues. Any surface measurements of temperature with one or more temperature monitors 810 may be insulated from the cooling layer of water to prevent the probe from being dominated by the cooling layer's effect. In one embodiment, the temperature of the patient's body is monitored. One or more temperature sensors 810 are placed between the cooling layer and the body, so the temperature sensor 810 can read the body temperature and/or the cooling layer temperature. There can be several ways that the temperature sensor can be isolated from the temperature of the cooling layer. A layer of insulation can be placed between the cooling layer and each temperature sensor 810. In such instances, the area around each temperature sensor 810 can receive less or no cooling.

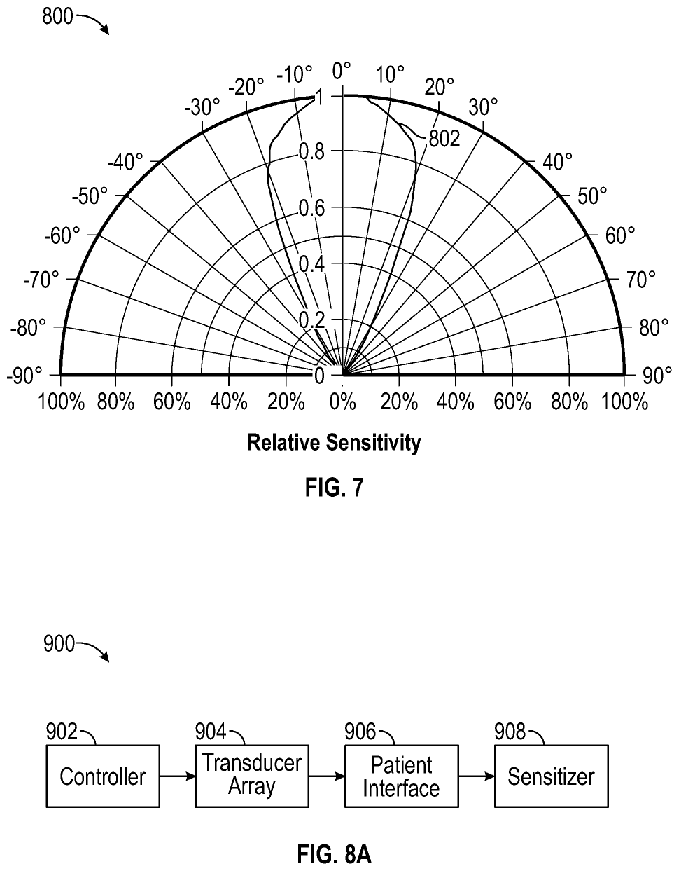

In various embodiments, a temperature monitor 810 is a temperature monitoring device, a temperature sensor, a temperature probe, an optical thermal sensor, a thermocouple, a thermometer, or other temperature measuring device. FIG. 7 is a chart 800 showing the relative sensitivity plot 802 of an infrared (IR) temperature sensor 810, according to at least one embodiment of the present disclosure. As shown in FIG. 7, a temperature probe 810 that measures only in one direction (e.g., unidirectional) can be utilized. An example of a unidirectional temperature sensor 810 can be an IR temperature sensor. IR temperature sensors 810 measure the infrared light being emitted by an object via black body radiation. IR temperature sensors 810 accept radiation coming in from a small range of angles (e.g., an acceptance cone). In this application, one or more IR sensors 810 can be oriented so that the cone of acceptance of each sensor can be facing the patient's body. One or more methods above can be combined to accurately monitor the temperature of the patient's body.

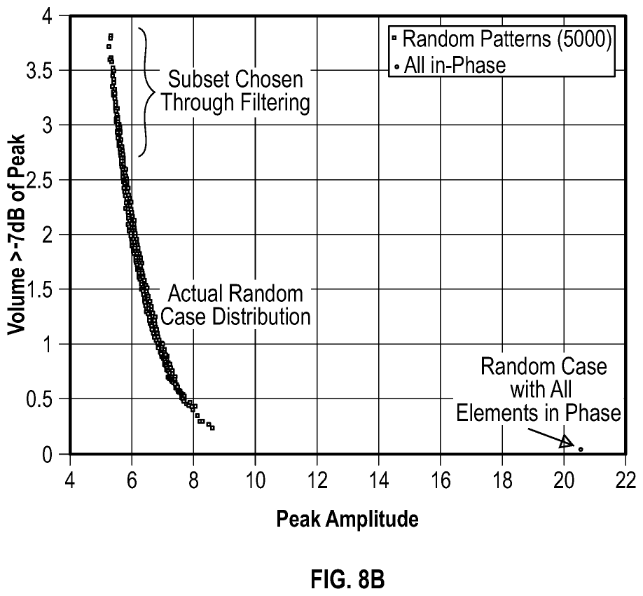

FIG. 8A is a block diagram of a ultrasound transducer system 100, wherein the ultrasound transducer system 100 is a non-invasive sonodynamic therapy system 900, according to at least one embodiment of the present disclosure. The non-invasive sonodynamic therapy system 900 comprises a controller 902 coupled to an ultrasonic transducer array 904 to control the operation of the ultrasonic transducer array 904 to generate a suitable ultrasonic acoustic wave. The ultrasonic transducer array 904 is coupled to a patient interface 906 to couple the ultrasonic acoustic wave produced by the ultrasonic transducer array 904 to a sensitizer 908 that accumulates in tumor cells within the patient's body. In one embodiment, through a process called sonoluminescence, the ultrasonic acoustic wave produces light that activates the sensitizer 908 and causes necrosis of the tumor cells. In one embodiment, an ultrasound acoustic wave produces light through a process called sonoluminescence. Sonoluminescence can occur when the ultrasound acoustic wave collapses fluid bubbles causing cavitation and produces light in the process. The production of light happens far away from the ultrasonic transducer. The light produced through sonoluminescence activates protoporphyrin IX (PpIX) to produce ROS. Sonoluminescence can occur anywhere the intensity of the ultrasound acoustic wave is sufficient, which allows sonodynamic therapy to treat much deeper than photodynamic therapy. The ROS species cause oxidative stress which results in the cancer cell undergoing programmed cell death (apoptosis). In one embodiment, ultrasound acoustic sonication causes cavitation and microbubble generation, the collapse of which generate photons within the tissue. In one embodiment, the photons activate sensitizers such as 5-aminolevulinic acid (5-ALA) and/or protoporphyrin-IX, thereby treating tumorous or other undesired tissue. Photons may have wavelengths between about 250-750 nm, 300 nm-700 nm, 400-800 nm and values and ranges therein.

Sonodynamic therapy treatment employs a sensitizer 908 drug that only become cytotoxic upon exposure to ultrasound. Upon activation, sonodynamic therapy drugs generally referred to as “sonosensitizers” produce ROS that generate the cytotoxic effect to kill the tumor cell. Sonodynamic therapy provides much greater tissue depth that can be reached non-invasively by ultrasound as compared to photodynamic therapy (using light alone). In one embodiment, the sensitizer 908 may comprise 5-aminolevulinic acid (5-ALA), protoporphyrin IX (PpIX), hematoporphyrin, Rose Bengal, curcumin, titanium nanoparticles, chlorin e6, pheobromide-a, ATX-S10 (4-formyloximethylidene-3-hydroxy-2-vinyl-deuterio-porphynyl(IX)-6,7-dia spartic acid), photofrin, photofrin II, DCPH-P-Na(I), NPe6 (mono-I-aspartyl chlorin e6), polyhydroxy fullerenes, hypocrellin-B, ZnPcS₂P₂, methylene blue, sinoporphyrin sodium, Hexaminolevulinate (HAL), and any combinations and derivatives thereof.

In one embodiment, the sensitizer is administered to the patient orally. In one embodiment, the sensitizer is administered to the patient through routes other than intravenously and/or other than topically. In one embodiment, the sensitizer is administered to the patient via injection. In several embodiments, one, two or more sensitizers (such as 5-ALA alone or combined with another compound) are administered to a subject orally, intratumorally, topically, intravenously, and/or intrathecally. Ear or nasal drops and/or inhalation of one or more sonosensitizers is provided in some embodiments. Oral doses may include sublingual doses. In some embodiments, one, two or more agents (such as 5-ALA) that enhance or potentiate a sensitizer is administered with the sensitizer (before, after or simultaneously with the sensitizer). Examples of such agents include but are not limited to vitamins (such as vitamin D3), tetracycline antibiotics (such as doxycycline, minocycline, etc.), deferoxamine, calcitriol, gefitinib, metformin and imiquimod and methotrexate. 5-ALA and iron chelator(s) are used in one embodiment. In some embodiments, one or more sonosensitizers (such as 5-ALA) are administered (e.g., orally) to a patient without imaging the location of the sonosensitizer(s) or its products and/or metabolites (such as protoporphyrin IX (PpIX)) for, e.g., tumor location purposes. In one embodiment, one or more sonosensitizers (such as 5-ALA) is administered (e.g., orally) to a patient without using the sonosensitizer(s) or its products and/or metabolites (such as protoporphyrin IX (PpIX)) for diagnostic purposes (e.g., the administration of 5-ALA is therapeutic only).

In several embodiments, one or more sonosensitizers (such as 5-ALA) are administered orally to a patient and its products and/or metabolites (such as protoporphyrin IX (PpIX)) accumulates in tumor cells preferentially as compared to non-tumor cells. Ultrasound is then used after this accumulation. The oral dose may be in the form of capsules, tablets, caplets, pills, oral strips, sublingual forms, gels, liquids and powders (such as lyophilized powders that can be mixed with liquids such as water, saline, juice etc. for consumption by a patient) Liquicaps, liquitabs, and/or gel caps are used in some embodiments. In various embodiments, doses may be 1, 5, 10, 15, 20, 25, 30, 40, 50, 60, 70, 80, 90, 100, 200, 300, 400, 500, 600, 700, 800, 900, or 1000 mg per kg of patient body weight and any values and ranges therein, and may be divided into 2, 3 or more doses. Extended release and/or enteric coating compositions and formulations are provided in an embodiment. 5-ALA, taken orally, penetrates the blood-brain barrier in several embodiments. In various embodiments, a dosage of sonosensitizer is administered, or instructed for administration, 5, 10, 20, 30, 45, 60, 90, 120 minutes, 1, 2, 3, 4, 5, 6, 7, 8, 12, 24, 36, or 48 hours before a sonodynamic treatment.

In some embodiments, the sonodynamic process described herein may comprise injecting or otherwise administering microbubbles into the tumor tissue to “seed” cavitation, enabling bubble to accumulate in the tumor tissue, or injecting a drug to oxygenate tumor tissue. The sonodynamic therapy process described herein may be combined with one or more other adjuvant therapies such as chemotherapy, immunotherapy, radiotherapy, and/or HIFU. In some embodiments, ultrasound is used therapeutically to both act on PpIX (or another compound) and perform one or more therapeutic functions (such as additional effects on blood brain barrier, angiogenesis, vascularization, resistance to chemotherapy, metabolic pathways, etc.). HIFU, focused ultrasound, defocused ultrasound, unfocused ultrasound, sonic treatments, magnetism, electrodes, monopole, bipole, and/or tripole electric energy, light, lasers, fluorescence, and other photo/illumination and/or other forms of energy delivery, cryotherapy, or mechanical/surgical procedures may be used in connection with the ultrasound sonodynamic therapies disclosed herein. In some embodiments, only incoherent ultrasound is used herein to effect sonoluminescence. Non-invasive ultrasound is used in several embodiments that is extremal to a patient. In some embodiments, an ultrasound system or device that is at least partially implantable is used. In some embodiments, a device that is not implantable is used.

In one embodiment, 5-aminolevulinic acid (5-ALA), can be provided in any pharmaceutically acceptable formulation, and may be provided as the free acid, a pharmaceutically acceptable salt, or a pharmaceutically acceptable ester. In some embodiments, the 5-ALA is sterilized by for example irradiation or another sterilization process (such as gamma irradiation). In some embodiments, ultrasound is delivered to a subject several hours after a sensitizer (such as 5-ALA) is delivered to enhance efficacy (e.g., 1-24 hours, 1-5 hours, 2-4 hours, 4-10 hours, 6-8 hours, 5-9 hours, 1, 2, 3, 4, 5, 6, 7, 8, 9, 10, 12, 15, 24 hours and values and ranges therein). In various embodiments, a dosage of sonosensitizer is administered, or instructed for administration, 5, 10, 20, 30, 45, 60, 90, 120 minutes, 1, 2, 3, 4, 5, 6, 7, 8, 12, 24, 36, or 48 hours before a sonodynamic treatment. In one embodiment, multiple doses of a sensitizer (such as 5-ALA) are delivered periodically (with spacing between doses ranging from 1 minute to 1 hour, 1-6 hours, etc. as an example).

In one embodiment, 5-aminolevulinic acid (5-ALA) is an endogenous amino acid precursor to heme that is metabolized to fluorescent porphyrins, particularly protoporphyrin IX (PpIX). In one embodiment, 5-ALA is an endogenous amino acid precursor to the heme group (e.g., an iron coordination molecule within hemoglobin). In cancer patients, delivering 5-ALA systematically results in a preferential accumulation of protoporphyrin IX (PpIX) in the cancer tumor. In one embodiment, 5-ALA is lyophilized ALA reconstituted in a liquid (such as drinking water) and administered orally three to six hours before a treatment. In one embodiment, photodynamic treatment involves exposure of cancer tissue that is illuminated with violet light (400 nm to 410 nm) and the resulting PpIX fluoresces red light (635 nm). In one embodiment, the PpIX is a fluorescent label that improves neurosurgeons' ability to visualize brain tumors, especially the boundary layer with healthy tissue. Intravenous administration is provided in some embodiments.

In one embodiment, PpIX selectively accumulates in mitochondria of brain tumor cells. In one embodiment, PpIX selectively accumulates in mitochondria of brain tumor cells due to a reduced ferrochelatase activity in these cells, and differential expression of transporter proteins and several heme metabolic enzymes in glioma cells (e.g., cancer cells have an obstructed heme biosynthesis pathway). This results in the cancer cells becoming sensitized by the PpIX.

In some embodiments, to enhance stability and/or penetration, a sensitizer, including but not limited to 5-ALA, straight chain, unsubstituted alkyl 5-ALA esters, or PpIX, which demonstrate additional selectivity for tumor tissue are used. 5-ALA or its methyl or hexyl ester and/or branched alkyl 5-ALA esters and substituted benzyl 5-ALA esters may be used herein. Compounds having hydrolysable groups at carbon 4 of 5-aminolevulinic acid may be used in one embodiment. Hydrophobic sensitizers are used in some embodiments. For example, a sonosensitizer is provided within or on a surface of a microbubble in one embodiment. In one embodiment, a microbubble complex that includes a microbubble coupled to a sensitizer and another agent (such as an activating agent, a potentiator, a chemotherapeutic agent, etc.) is provided, which may be coupled directly or indirectly via a linker. In one embodiment, 5-ALA results in the accumulation of PpIX in cancer through one or more mechanisms. First, cancer cells preferentially transport 5-ALA through the cell membrane because of an overexpression of peptide transporter 2 (PEPT2). Secondly, PpIX accumulates because cancer cells have reduced expression of ferrochelatase (FECH), which completes the synthesis of the heme group. These two mechanisms result in PpIX growing in concentration in tumor cells while remaining low in health cells. In one embodiment, during sonodynamic therapy, PpIX behaves as a catalyst that converts molecular oxygen from a low-energy state into a higher energy state. These high-energy oxygen molecules are violently reactive and will damage cellular components. In particular, this reactive oxygen species (ROS) damages the mitochondria of cancer cells where the highest concentrations of PpIX occurs.

The non-invasive sonodynamic therapy system 900 may be employed to treat a variety of tumors and to treat the area around the tumor cavity, whether malignant or nonmalignant. The area around the tumor cavity includes cells that cause the recurrence and eventual mortality in malignant tumors. In one embodiment, the non-invasive sonodynamic therapy system 900 may be configured to treat prostate cancer via trans-rectal ultrasound sonodynamic therapy and cervical cancer via trans-vaginal ultrasound sonodynamic therapy, for example. In one embodiment, the treatment is direct to neuromodulation applications affecting the way neurons fire, such as spasticity, pain, tremor, migraine, and other neurological conditions or disorders associated with coordination or movement of the body. In some embodiments, the treatment is directed to nerves, nerve disease, Bell's palsy, cerebral palsy, epilepsy, Alzheimer's disease, memory disorders, Parkinson's disease, prion disease, multiple sclerosis, Motor neurone disease (MND), atherosclerosis (e.g., treatment with sonodynamic therapy for reducing atherosclerotic plaque inflammation in cardiovascular and peripheral artery disease), depression, anxiety, mood disorders, obsessive-compulsive disorder, tics, autism, neuropathic pain, psychiatric conditions, neurological conditions, tinnitus, sleep apnea (such as stimulation of hypoglossal nerve in a rhythm synchronized with the patient's breathing to treat obstructive sleep apnea), seizures, or other treatments (e.g., ablation, histotripsy, mechanical fractionation, cavitation, angiogenesis, immunomodulation, neuromodulation, and/or thrombolysis) and/or monitoring of the nervous system. In some embodiments, therapy treatment is directed to modulation of nervous system (including brain) activity via stimulation, activation, inhibition and/or modulation. In some embodiments, treatment is directed to brain regions for emotion, attention, arousal, concentration, and/or sleep.

In one embodiment, the controller 902 may be configured to drive the ultrasonic transducer array 904. The controller 902 may be configured to execute one or more than one control algorithm setup/reflection assessment and tune the drive frequency to skull thickness. This can be done automatically. In one embodiment, the control algorithm may be configured to pulse or control the “duty cycle” of the ultrasonic transducer array 904 drive waveform to generate high temporal peak acoustic intensity of ultrasonic acoustic waves with low temporal average acoustic intensity sufficient to activate the sensitizer 908 while preventing thermal necrotic death of the tumor cells in the treatment region. In one embodiment, the control algorithm may be configured to pulse or control the “duty cycle” of the ultrasonic transducer array 904 drive waveform to generate high pulse peak acoustic intensity of ultrasonic acoustic waves with low pulse average acoustic intensity sufficient to activate the sensitizer 908 while preventing thermal necrotic death of the tumor cells in the treatment region. In another embodiment, the control algorithm may be configured to generate packets of waves that are delayed to overlap the tumor. In another embodiment, the control algorithm may be configured to control the intensity of the ultrasonic acoustic wave.

In another embodiment, the control algorithm may be configured to control the phase of the ultrasonic acoustic wave, In another embodiment, the control algorithm may be configured to randomize the phase of the ultrasonic acoustic wave. Modulating acoustic waves with phase randomization promotes broad consistent coverage across a treatment region where acoustic wavefronts constructively combine at varying pseudo random locations within the treatment region, rather than the exact same location with each cycle. This control scheme provides a more homogeneous treatment region to aid broad consistent treatment coverage and avoid sub therapeutic dead spots in the treatment region. Phase randomization provides additional benefit in adapting to the treatment environment. Repeating the exact same excitation pattern in some types of acoustical environments could lead to the potential for standing waves to form. Several embodiments herein overcome certain limitations of standing waves, which can sometimes deliver unintended treatment energy to the patient. A controller scheme that provides phase randomization of the acoustic waveform can mitigate the risks of repetitive excitation that can lead to standing waves.

In one embodiment, a unique waveform phase is randomly applied to each transducer element, and that unique phase selection is re-randomized with each subsequent pulse. In one embodiment, the phase selection is not randomized, rather it is intentionally prescribed from a set of phase combinations. One purpose of prescribing a set of unique phases is to produce a broader and more uniform ultrasound field by avoiding combinations that create unwanted peaks in the field, thereby creating a larger and more homogenous therapeutic field. One reason for selecting a particular set (rather than a random set) of unique phases for each element is that some random patterns can produce unwanted areas of coherence, or localized peaks. When therapy is calibrated to these localized peaks it can reduce the overall ensonification volume. Eliminating or reducing these unwanted areas of coherence can create a larger and more homogenous ensonification volume. In one embodiment, this approach involves a filtering step to prescribe unique phasing for each element in the array. In one embodiment, a control algorithm may be configured to draw unique phase combinations and permutations from a list of pre-screened phase sets, selected via hydrophone measurement and/or simulations and/or analytical analysis of disorder to produce particularly incoherent ultrasound fields. In one embodiment, a control algorithm may be configured to draw unique phase combinations and permutations from a list of pre-screened phase sets, selected via hydrophone measurement and/or simulations and/or analytical analysis of disorder to produce particularly incoherent ultrasound fields such that the volumetric field can be enlarged (e.g., by 20%, 30%, 40%, 50%, 60%, 70%, 80%, 90%, 100%, 150%, 200% or more) and/or maximized. FIG. 8B illustrates an embodiment with this approach, and for illustrative purposes assumes a goal of creating a large volumetric field. In one embodiment, the goal is creating the largest possible volumetric field. In one embodiment, the volumetric field is generated to produce a volume that is within −2 dB to −15 dB (e.g., −2, −3, −4, −5, −6, −7, −8, −9, −10, −11, −12, −14, and −15 dB) of the peak pressure. In various embodiments, In one embodiment, the volumetric field is generated to produce a volume that is within −2 dB to −15 dB which corresponds to a pulse average of 1-20 W/cm² (e.g., 1-18, 1-10, 1-15, 2-15, 2-10, 3-15, 5-15, 10-15 W/cm², and ranges and values therein) across a large therapeutic volume. For example, if each of the elements in the array share the same or similar phase, a −7 dB field volume will be extremely small and focused. If unique phases for each element are randomly selected, the −7 dB therapeutic volume can vary considerably across a distribution of volumes. If a subset of unique phase combinations for each element are selected through filtering (via measurement, simulations, and/or analytical analysis), the resulting therapeutic volume within −7 dB of the peak pressure location can be significantly maximized. The y-scale volume unit illustrated is mL, and the x-scale amplitude is MPa of pressure. The drive capability of the amplifier output can be controlled to increase or decrease pressure to accomplish any desired peak pressure. In this example, a “maximum coherence” selection of randomly selected unique phases for each element would generate a −7 dB volume less than 0.5 mL, whereas a set of unique phase combinations for each element can increase that −7 dB therapeutic volume to greater than 3.5 mL (an increase of 700%). In various embodiments, the increase in therapeutic volume using selected unique phases instead of allowing for maximum coherence is in a range between 10% and 2,000% (e.g., 10, 25, 40, 50, 60, 75, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, and 2000% including values and ranges therein).

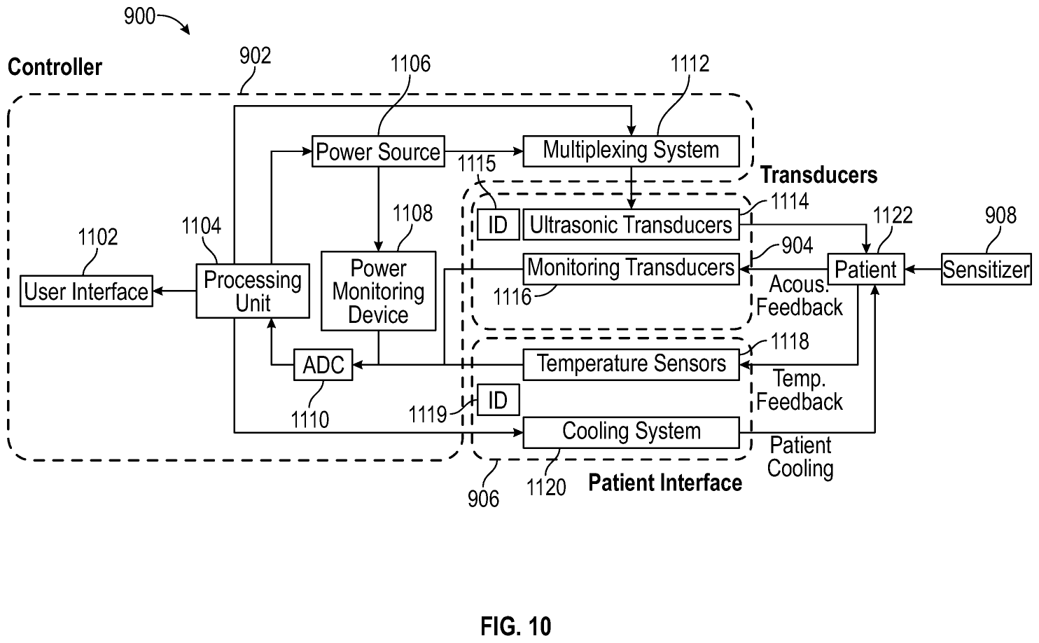

A feedback loop may be provided back to the controller 902 to adjust the drive signal to the ultrasonic transducer array 904 based on in situ variables such as tissue depth, tissue thickness, tissue volume, skull thickness, temperature, among other variables. In one embodiment, the controller 902 may be located in an ultrasonic generator or may be located elsewhere. In various embodiments, in situ variables may include a disease state or an inner body location. The disease state may include alternative treatment ultrasonic transducer probe that is driven differently for each disease state. Examples of feedback loops are described hereinbelow in connection with FIGS. 10-12.

In one embodiment, the ultrasonic transducer array 904 may be configured according to the transducers 150, 400, 450 described hereinabove. In various embodiments, however, the form factor of the ultrasonic transducer array 904 may be configured to couple ultrasonic acoustic waves in various locations on the patient's body other than the head. For example, the ultrasonic transducer array 904 may be configured to generate ultrasound that activates a sensitizer 908 to treat tumors in the brain, such as glioblastoma, spine, lung, breast, mouth, tongue, stomach, liver, pancreas, intestines, rectum, colon, vagina, ovary, testes, leukemia, lymphoma, among others, whether the tumors are malignant or nonmalignant.

In various configurations, the ultrasonic transducer array 904 is non-invasive and produces ultrasonic acoustic waves capable of reaching the target tumor cells non-invasively. As described hereinabove, the ultrasonic transducer array 904 may be configured as annular array, 2D grid array, a linear array, and the like, to generate an adaptively focused ultrasonic acoustic wave optimized based on in situ variables such as tissue depth, tissue thickness, tissue volume, skull thickness, among other variables. In other embodiments, the ultrasonic transducer array 904 may adaptively focus or adjust the ultrasonic acoustic wave based on pretreatment planning or safety. In one embodiment, the controller 902 executes a control algorithm to generate selectively convergent/divergent ultrasonic acoustic waves including adaptive focus for collaborative transducer performance. The ultrasonic acoustic array 904 may be configured to perform transmitter and receiver functions that may be controlled by the controller 902.

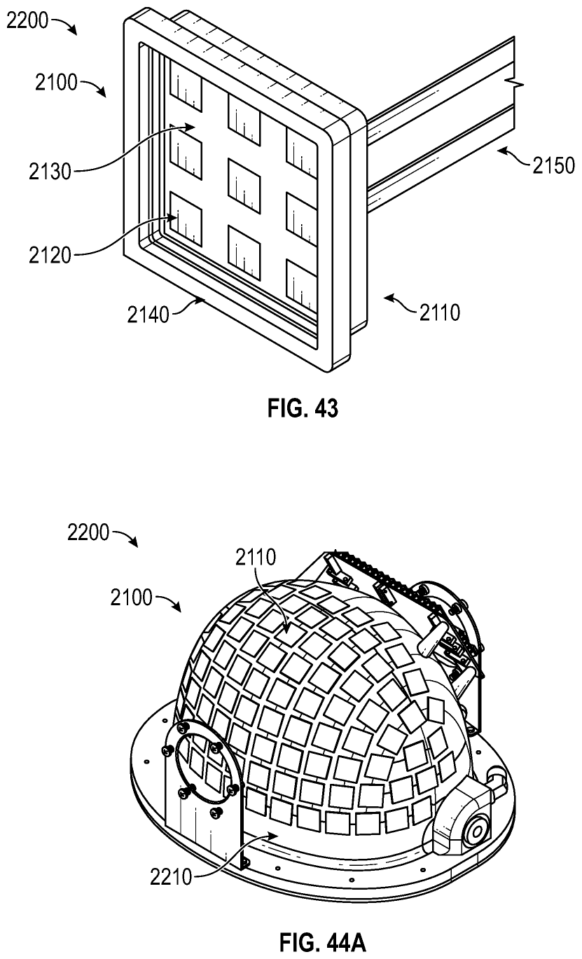

FIG. 43 illustrates an embodiment of an ultrasonic transducer array 2100 configured for applying sonodynamic therapy to a treatment volume, such as a brain, a portion of a brain, or an entire brain hemisphere. In several embodiments, the ultrasonic transducer array 2100 is constructed out of multiple sub-arrays 2110. In one embodiment, a sub-array 2110 may be constructed as a discrete module, including a grid of flat piezo-electric elements 2120 mounted within a frame structure 2140 that includes an acoustic matching layer for the piezo-electric elements, a dielectric layer for patient isolation, and one or more flex strip circuits 2150 or other electrical provisions for connectivity with an ultrasound drive system. In one embodiment, an individual sub-array 2110 includes a 3×3 grid of 9 elements 2120 arranged in a polygonal, quadrilateral, rectangular, square, rhombus, or diamond shaped configuration as illustrated in FIG. 43. In one embodiment the sub-array 2110 can include various numbers of elements in arranged in various rows, columns, or other geometric layout locations (e.g., 1, 2, 3, 4, 5, 6, 7, 8, 9, 10, 11, 12, 13, 14, 15, 16, 18, 20, 21, 24, 25 or more elements 2120). One benefit of creating a transducer array 2100 out of multiple sub-arrays 2110 is that the sub-arrays 2110 can be packed into a frame 2140 with complex geometry to create a resultant overall array with complex geometry that otherwise could be very difficult to fabricate.

In one embodiment, the sub-array 2110 is configured for packing into a frame structure. In one embodiment the frame structure has a complex geometry. In one embodiment, the sub-array 2110 includes a hermetically sealed and electrically isolated components that include one or more piezo-electric elements 2120, an acoustic matching layer, a frame 2140 and one or more flex circuits 2150 or other electrical interface. In one embodiment, spacing 2130 between elements 2120 may be large with air or epoxy filled kerf.

FIGS. 44A, 44B and FIG. 45 illustrate embodiments of a sonodynamic treatment system 2200 with a transducer array 2100 arranged into a helmet structure for applying sonodynamic therapy to a brain, a portion of a brain such as an entire brain hemisphere or a portion of a brain hemisphere. FIG. 44A and FIG. 44B illustrate an embodiment of a helmet frame structure with complex geometry that is contoured to be approximately congruent with a shape of a human head. In one embodiment, several sub-arrays 2110 are mounted into the frame structure resulting in an overall densely packed transducer array 2100 that is contoured to approximately match the shape of the head.

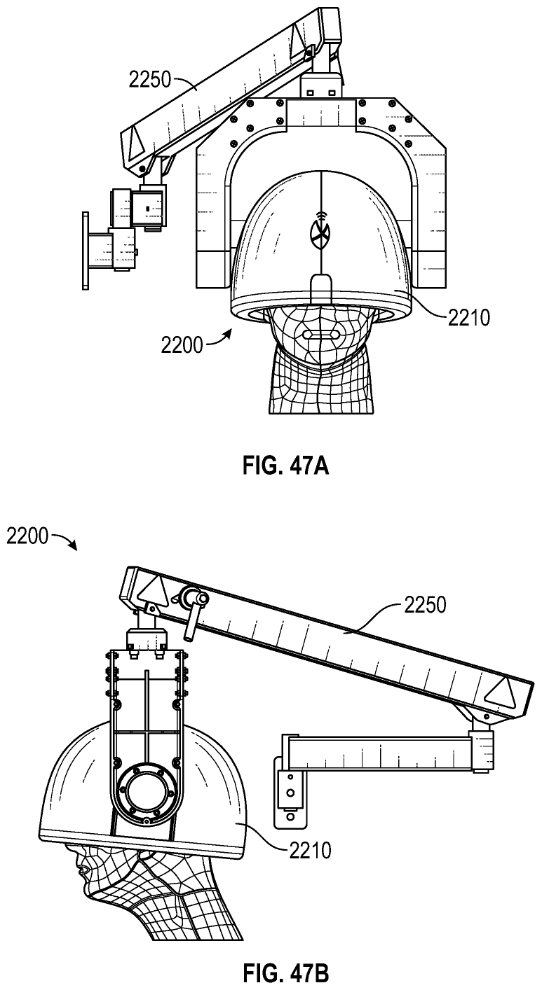

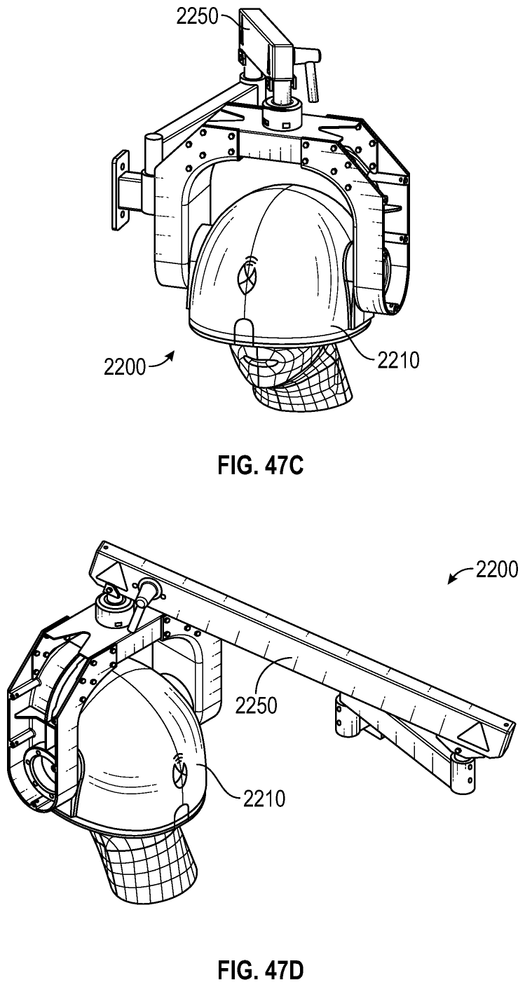

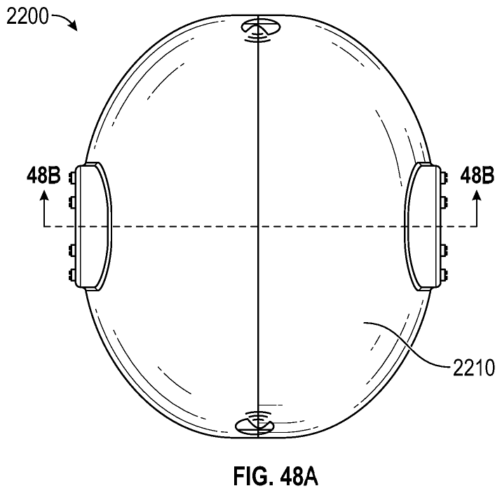

FIGS. 45, 47A-D, and 48A-C illustrate an embodiment of a sonodynamic treatment system 2200 with a transducer array 2100 with complex geometry that can be incorporated into a helmet structure 2210 that is couplable to a patient's head. In one embodiment, the sonodynamic treatment system 2200 may arrange the position of the helmet structure 2210 with a support arm 2250. The coupling with the patient can in one embodiment be aided by a fluid filled membrane that lines the interior surface of the helmet structure 2210, whereby the fluid level in the membrane can be increased or decreased to accommodate varying head sizes. The fluid filled membrane can also include fluid circulation that aids in dissipating heat generated by the ultrasound therapy. FIGS. 44A-46 illustrate an embodiment for creating a large transducer array 2100 with complex geometry that targets broad treatment of an entire or portion of an organ, such as a diseased brain hemisphere. The shape of the human head represents a complex geometry with many different variable radii, arc lengths, and contours. In some cases it is not possible, practical, or feasible to create a transducer array 2100 that precisely mirrors the complex shape of the human head, or other complex organ geometries, by means of traditional transducer array fabrication, such as monolithic ceramic structures. In some embodiments utilizing various sub-arrays 2110 as modular components that can be fabricated and then mounted into a frame structure 2210 such as a helmet structure 2210 defining a desired complex shape, produce a transducer array 2100 with complex geometry that would otherwise be difficult to accomplish. In various embodiments, the overall number of sub-arrays 2110 and mounting locations in the frame structure 2210 can be optimized to produce a desired large, homogenous ultrasound field that saturates an entire diseased organ, for example a brain hemisphere, for broad initiation of sonodynamic therapy across the entire tissue volume or organ. In one embodiment the grid spacing, packing density and/or arrangement between elements 2120 in an individual sub-array 2110 is optimized to result in approximately equal element spacing across a portion of, or the entire, transducer array 2100. In one embodiment, equal element spacing across the array 2100 can be beneficial for balancing energy to generate a substantially homogenous therapeutic field. In one embodiment the grid spacing, packing density and/or arrangement between elements 2120 in an individual sub-array 2110 is optimized with varied element spacing across a portion of, or the entire, transducer array 2100. In one embodiment, varied element spacing across the array 2100 can be beneficial for balancing energy to generate a substantially homogenous therapeutic field.

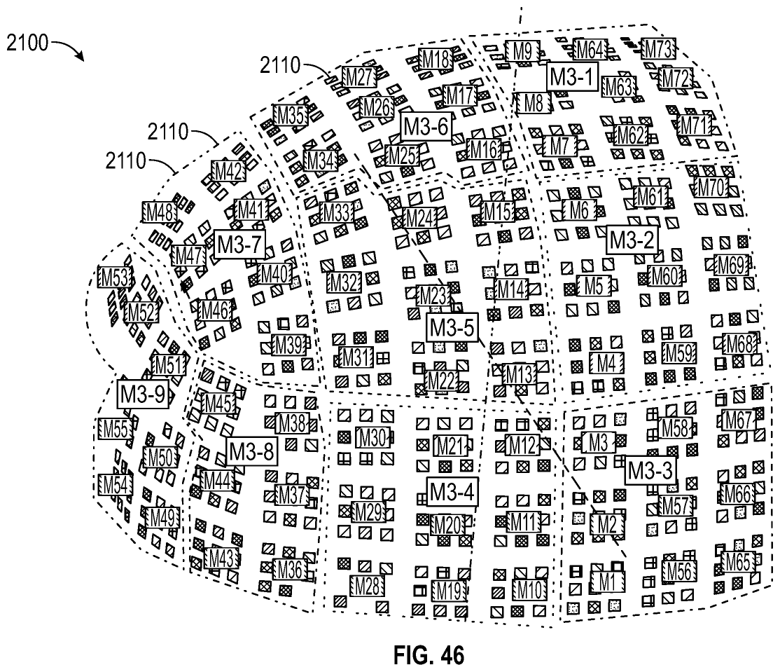

FIG. 46 illustrates an embodiment of a method of increasing the size and energy distribution of a therapeutic treatment field while simultaneously suppressing heating by using scanned sub-apertures of an array. In one embodiment, a portion of the individual sub-arrays 2110 from the helmet structure 2210 of FIG. 45 is grouped together into nine sub-apertures (e.g., M3-1, M3-2, M3-3, M3-4, M3-5, M3-6, M3-7, M3-8, M3-9) as an illustrative example. Each of the sub-apertures (M3-1, M3-2, M3-3, M3-4, M3-5, M3-6, M3-7, M3-8, M3-9) can be fired at different times, and with different delays, and amplitudes across the aperture intended to steer energy deeper into the tissue, and broader across the tissue. Furthermore, the combination of sub-arrays 2110 that make up each sub-aperture can be continually redistributed with each subsequent pulse, thereby creating a sequence of many different sub-aperture combinations that continually, and uniquely redistributes the field rather than pulsing the same sub-arrays and sub-apertures on a repeat basis. Furthermore this technique can mitigate heat generation that may develop when firing either all sub-arrays and sub-apertures, or the same selection of sub-arrays and sub-apertures on a repeated sequence. In one embodiment, sub-arrays are arranged into different groupings of sub-apertures. The selection of sub-arrays that create sub-apertures can vary with each pulse resulting in many different sub-aperture firings over time. The sub-apertures can include different tunings (delays, amplitude, apodization etc.) that helps to continually redistribute the energy thereby creating a broader and deeper field. The sub-aperture firing technique can also be used as means to sequence which elements of the overall array are active during any given burst as a means to reduce and distribute unwanted heat generation.

FIGS. 47A-47D are illustrative of a sonodynamic treatment system with a support arm of the ultrasonic transducer array according to an embodiment. FIGS. 48A-C are illustrative of a sonodynamic treatment system with a helmet structure with the ultrasonic transducer array according one embodiment. FIG. 48A illustrates atop view of the helmet structure. FIG. 48B shows a cross section of the helmet structure FIG. 48A. FIG. 48C shows a cross section of the helmet structure of FIG. 48B.

In one embodiment, the ultrasonic transducer array 904 is coupled to the patient interface 906 to facilitate acoustic coupling of the ultrasonic vibrations generated by the ultrasonic transducer array 904 into the patient's body. The patient interface 906, like the ultrasonic transducer array 904, is non-invasive. In one embodiment, the patient interface 906 may be configured to remove air between the ultrasonic transducer array 904 and the patient's body to facilitate acoustic coupling. In one embodiment, the patient interface 906 may be configured to remove excess heat from the patient's body. In some configurations, the patient interface 906 may comprise a variety of sensors, such as any one or more of a temperature sensor 810, a reflection monitor 820, a cavitation monitor 830, an imaging device 840, an internal alignment device 850, and/or external alignment device 850, and combinations thereof. Signals from such sensors may be provided as feedback to the controller 902 (see FIG. 10 for example). Such feedback may be employed to control the ultrasonic transducer array 904 to generate a desired ultrasonic acoustic wave. The patient interface 906 also may include gel or hydrogel layers to improve the acoustical coupling between the ultrasonic transducer array 904 and the patient's body. In one embodiment, the patient interface 906 may be configured to locally apply cooling. In one embodiment, the patient interface 906 may be configured for sensor feedback to the processing unit with a controller 902.

Finally, the non-invasive sonodynamic therapy system 900 comprises a sensitizer 908 that may be absorbed by the tumor cells. In one embodiment, sonodynamic therapy may include the combination of the sensitizer 908, such as a sensitizing drug, ultrasound generated by the ultrasonic transducer array 904 coupled into the patient's body by the patient interface 906, and molecular oxygen. Although these components are non-toxic individually, when combined together, a cytotoxic ROS is generated to kill the tumor cells. Sonodynamic therapy may be configured to provide penetration of ultrasound through the patient's body and can be used to treat a wide array of deep and hard to access tumors.

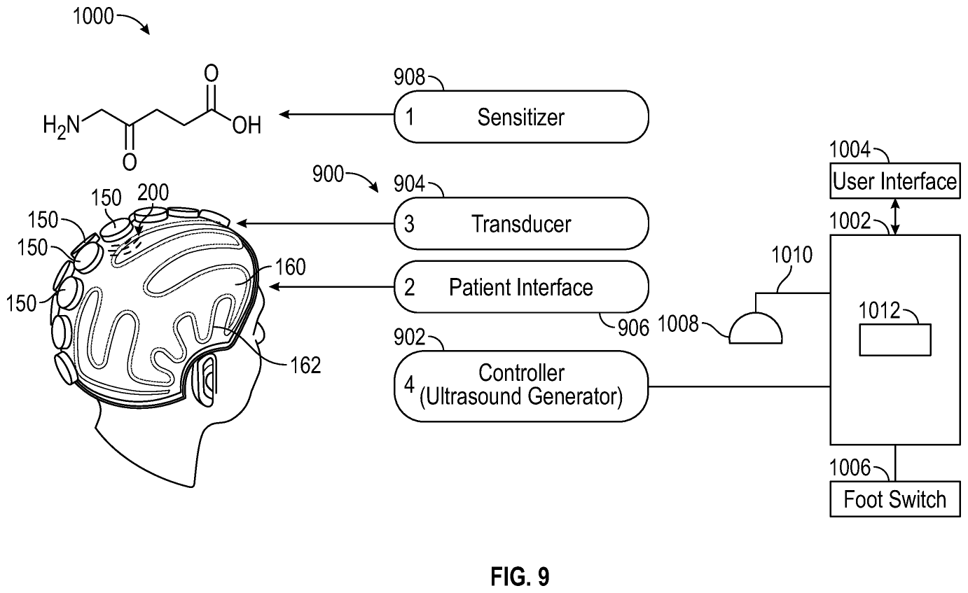

FIG. 9 is an illustrative diagram 1000 of the sonodynamic therapy system 900 shown in FIG. 8A, according to at least one embodiment of the present disclosure. In one embodiment, the sonodynamic therapy system 900 comprises a controller 902 that may be located in an ultrasonic generator 1002. The ultrasonic generator 1002 comprises a controller 1012, a user interface 1004, a switch (e.g., hand or foot switch) 1006 for activating the controller 1012, and a cap or helmet 1008 that is placed over the head of the patient. A cable 1010 that carries electrical signals to and from the ultrasonic transducer array 904 couples the transducer array 904 and the ultrasonic generator 1002. The ultrasonic transducer array 904 comprises an array of ultrasonic transducers 150, 400, 450 placed over a patient interface 906 such as the skull cap 160. The ultrasonic generator 1002 drives the ultrasonic transducers 150, 400, 450 to generate an ultrasonic acoustic wave 200 that is coupled into the body of the patient to excite the sensitizer 908 ingested by the patient and absorbed by the tumor cells. The controller 1012 shapes the acoustic wave to achieve a convergent, divergent, or planar acoustic wave, or more complex acoustic waves. In one embodiment the sensitizer 908 may comprise and ALA sensitizing drug that is activated in a sonoluminescence process, for example.

FIG. 10 is a schematic diagram of the sonodynamic therapy system 900 shown in FIGS. 8A, 9 and 10 according to at least one embodiment of the present disclosure. The controller 902 of the sonodynamic therapy system 900 comprises a user interface 1104 coupled to a processing unit 1104 and configured to receive input from a user and providing output to the user. The processing unit 1104 may be a processor or microcontroller coupled to a memory (e.g., memory circuit), a control circuit, or a combination thereof. The ultrasonic transducer array 904 comprises one or more than one ultrasonic transducer 1114 and one or more than one monitoring ultrasonic transducer 1116. It will be appreciated that the same ultrasonic transducer element may be configured to implement an ultrasonic transmitter function as well as a receiver function (see FIG. 12 for example). The patient interface 906 comprises one or more than one temperature sensors 810, 1118 to monitor the temperature of the patient 1122. The patient interface 906 also comprises a cooling system 1120 to reduce the temperature of the patient 1122. In one embodiment, the patient interface 906 may be configured to eliminate air gaps between the transducer 1114 and the patient 1122 to enable acoustical coupling.

The processing unit 1104 is configured to execute machine executable instructions to implement various control algorithms as described herein. The processing unit 1104 may comprise a memory to store such machine executable instructions and processing engines to execute the control algorithms. The processing unit 1104 also may be implemented in hardware with digital and analog electronic components. The processing unit 1104 is coupled to a multiplexing system 1112 and a power source 1106 suitable for driving the ultrasonic transducers 1114.

The ultrasonic transducers 1114 are coupled to the body of the patient 1122 to activate the sensitizer 908 administered to the patient 1122. In one embodiment, at least one sonosensitizer 908 agent may be configured for preferential accumulation in selective tissue of the patient 1122. Monitoring ultrasonic transducers 1116 monitor acoustic feedback from the patient 1122 and generate signals that are provided as feedback to the processing unit 1104 via an analog-to-digital converter 1110 (ADC). In addition to the acoustic feedback, a power monitoring device 1108 monitors the power source 1106 and provides feedback to the processing unit 1104 through the ADC 1110. The processing unit 1104 controls the ultrasonic transducer drive signals based on the acoustic feedback signal and/or the power monitoring signal to achieve a desired ultrasonic acoustic wave inside the body of the patient 1122. In one embodiment, at least one ultrasonic transducer 1114 is configured to output selectively convergent and divergent acoustic waves. The transducer 1114 may be configured in an annular array or a grid array. The transducer 1114 may be configured with multiple electrodes. The transducer 1114 may be configured to receive reflected acoustical signals.

The processing unit 1104 is coupled to the temperature sensors 810, 1118 and receives patient temperature feedback through the ADC 1010. The processing unit 1104 controls the cooling system 1120 based at least in part on the patient temperature feedback signal.

In one embodiment, the processing unit 1104 is configured to produce a pulsed acoustical signal with temporal-average intensity output of 30 W/cm², 25 W/cm², 20 W/cm², 15 W/cm², 10 W/cm² and below, below 8 W/cm² (e.g., 7.0 W/cm², 6.5 W/cm², 6.0 W/cm², 5.5 W/cm², 0.05 W/cm², 4.5 W/cm², 4.0 W/cm², 3.5 W/cm², 3.0 W/cm², 2.5 W/cm², 2.0 W/cm², 1.5 W/cm², 1.0 W/cm², 0.5 W/cm², 0.4 W/cm², 0.3 W/cm², 0.2 W/cm², 0.1 W/cm², 0.05 W/cm²), and any values and ranges therein, such as 1-30, 1-20, 1-10, 2-30, 2-20, 2-25, 2-20, 2-15, 2-10, 2-5, 5-30, 5-25, 5-20, 5-15, 5-10, 10-30, 10-25, 10-20, 10-15 W/cm² and values therein. In one embodiment, the processing unit 1104 is configured to produce a pulsed acoustical signal with pulse-average intensity output of 30 W/cm², 25 W/cm², 20 W/cm², 15 W/cm², 10 W/cm² and below, below 8 W/cm² (e.g., 7.0 W/cm², 6.5 W/cm², 6.0 W/cm², 5.5 W/cm², 0.05 W/cm², 4.5 W/cm², 4.0 W/cm², 3.5 W/cm², 3.0 W/cm², 2.5 W/cm², 2.0 W/cm², 1.5 W/cm², 1.0 W/cm², 0.5 W/cm², 0.4 W/cm², 0.3 W/cm², 0.2 W/cm², 0.1 W/cm², 0.05 W/cm²), and any values and ranges therein, such as 1-30, 1-20, 1-10, 2-30, 2-20, 2-25, 2-20, 2-15, 2-10, 2-5, 5-30, 5-25, 5-20, 5-15, 5-10, 10-30, 10-25, 10-20, 10-15 W/cm² and values therein. In various embodiments, acoustic intensity is produced from each active element in an array. In one embodiment, an intensity is produced from one or more active elements in the array. The processing unit 1104 is adapted to apply amplitude-modulated acoustical signals including constructive interference over a plurality of wave cycles. The processing unit 1104 further may be configured to output packets of acoustic waves at various delayed sequences to provide diffused tissue coverage. The processing unit 1104 may be configured to execute frequency adaptive algorithms to optimize transmission of acoustical signals. The processing unit 1104 may be configured to control phased randomization of acoustical signals.

In various embodiments, the present disclosure provides a sonodynamic therapy device comprising a transducer 904, a patient interface 906, and a controller 902 adapted to activate a sensitizer 908 within the body of the patient 1122. The transducer 904 may comprise one or more than one transducer 1114, 1116 where the controller 902 is configured to generate a broadband range of ultrasonic frequencies to drive the transducer 904 and produce divergent, convergent, or planar acoustic waves.

In one embodiment, the patient interface 906 is configured to transmit acoustic waves produced by the transducer(s) 904 into the body of the patient 1122 thus acoustically coupling the transducer(s) 904 to the patient 1122. In one embodiment, the patient interface 906 provides a cooling system 1120 to remove any excess heat that builds up in the patient 1122 as a result of the coupling acoustic energy to the body of the patient 1122. In one embodiment, the patient interface 906 may comprise an integral cooling system 1120. The patient interface 906 may comprise a hydrogel cap filled with gel or a water-filled cap with cooling channels. In one embodiment, the patient interface 906 comprises one or more than one sensor 1118 to provide feedback to the processing unit 1104 of the controller 902. The sensors 1118 may include, for example, temperature sensors 810, optical temperature sensors 810 to measure temperature in a particular direction, acoustic sensors, which may include the same transducers 904 used for transmitting acoustic signals. The patient interface 906 may be configured to remove air from the patient interface 906 to improve acoustic coupling between the transducer 904 and the body of the patient 1122. In another embodiment, the patient interface 906 may be configured to cool the patient 1122. In yet another embodiment, the patient interface 906 may be configured to cool the transducers 904, for example, to keep the transducers at the same temperature to achieve frequency stability.

In one embodiment, the patient interface 906 may be adapted and configured to fit various patient anatomies. For example, the patient interface 906 may be adapted and configured to fit patient anatomies for sonodynamic therapy specifically adapted to treat tumors located in the brain, lung, breast, stomach, liver, pancreas, intestines, rectum, colon, vagina, testes, among others, for example. A sonodynamic therapy device may be adapted to wrap around the torso or limb of the patient and/or employed to treat osteosarcoma into the bone. The controller 902 may be adapted to detect either the patient interface 906 or the sonodynamic therapy device such as the transducer 904 or patient interface 906 and select a treatment algorithm to produce acoustic waves optimized for treating the various tumors. The transducer 904 or patient interface 906 may be identified using identification (ID) circuits 1115, 1119 comprising a single-wire serial EEPROM, for example. The ID circuit 1115, 1119 EEPROM may contain both a preprogrammed unique serial number and memory sections. Any or all of the memory sections can be permanently locked by the end-equipment manufacturer to allow tracking of products and identifying attachments. Other identification techniques may include detecting the impedance of the transducer 904 or patient interface 906 and associating the impedance with a treatment algorithm.

In one embodiment, the controller 902 is configured to generate electrical drive signals to actuate one or more than one ultrasonic transducer 904 to produce an acoustic wave to activate a sensitizer 908 located within the body of the patient 1122. In one embodiment, the electrical drive signals generated by the controller 902 may actuate the one or more than one ultrasonic transducer 904 to produce acoustic waves of varying intensities, amplitudes, or frequencies. In another embodiment, the acoustic waves may be amplitude modulated, frequency modulated, phase modulated, continuous, discontinuous, pulsed, randomized, or combinations thereof. In other embodiments, the acoustic waves may be produced in a packet of wave cycles, where the number of cycles per packet may be predetermined to achieve a desired outcome that is different from a focused ultrasound pulse, for example. In other embodiments, the controller 902 is configured to generate a frequency modulation signal to produce a frequency-modulated acoustic wave. In one embodiment, the controller may be configured to generate an intra or inter pulse variation signal that can be used to reduce standing acoustic waves.

In one embodiment, the controller 902 is configured to apply an amplitude-modulated acoustic ultrasound signal which constructively interferes over a plurality of wave cycles. In one embodiment, the intensity of each of the plurality of acoustic waves remain within a safe range wherein the ultrasound energy carried by each of the plurality of acoustic waves is safe to the tissue of the patient 1122, such as the brain or other body part. In one embodiment, the controller 902 may be configured to drive the transducer 904 to generate an amplitude-modulated acoustic wave which produces a constructive wavefront. In one embodiment, ultrasound modifies the blood brain barrier (BBB). In one embodiment, ultrasound facilitates delivery of a therapeutic agent such as a drug and/or sonosensitizer (e.g., genes, cells, stem cells, nucleic acids, growth factors, antibodies, etc.) across the blood brain barrier. In one embodiment, ultrasound energy causes vibrations to induce a temporary disruption to the blood brain barrier. In various embodiments, ultrasound therapy is used to temporarily allow for increased access through a blood brain barrier for treatments such as sonodynamic therapy, chemotherapy, improved mindfulness and meditation, hallucinatory effects, and recreational enhancement. In various embodiments, increased access through the blood brain barrier allows for exchange of materials in to and/or out of the brain via blood in blood vessels. In one embodiment, an ultrasound transducer system 100 is fitted to a patient head via a helmet or cap after using a previously acquired CT or MRI image of the head (e.g., brain, skull, etc.) an imaging device (camera, CT, or MRI or other imaging device) can be used during treatment to confirm alignment of the ultrasound transducer system 100 to the target treatment site in the brain. Optionally, microbubble additives can be administered to the patient to increase ultrasound activity and cavitation. In the treatment area of the ultrasound transducer system 100, low frequency/intensity ultrasound can cause cavitation and bubble forming to open or expand the blood brain barrier. In some embodiments, the blood brain barrier remains open or expanded for 1-48 hours. Additional therapies (immunotherapy, chemotherapy, sonodynamic therapy, and other therapies described herein) may be applied via the open and expanded blood brain barrier.

In one embodiment where the sonodynamic therapy device comprises one transducer 904 and the controller 902 may be configured to generate a drive signal to actuate the transducer 904 to produce a long acoustic ultrasonic wave packet. In one embodiment, the controller 902 may be configured to generate a drive signal to actuate the transducer 904 to produce an ultrasonic acoustic wave packet composed of a sinusoidal wave amplitude modulated by a Gaussian pulse. In another embodiment, the controller 902 may be configured to generate a drive signal to actuate the transducer 904 to produce an ultrasonic acoustic wave packet composed of a sinusoidal wave amplitude modulated by a rectangular pulse. In another embodiment, the controller 902 may be configured to generate a drive signal to actuate the transducer 904 to produce an ultrasonic acoustic wave packet composed of a sinusoidal wave amplitude modulated by a triangular pulse. The ultrasonic acoustic wave packet may comprise intra or inter wave packet variation. In one embodiment, the controller 902 may be configured to generate a drive signal to actuate the transducer 904 to produce an acoustic ultrasonic pulse. The acoustic wavefronts of the ultrasonic pulse may either converge to focus the ultrasonic energy to a specific region or diverge to spread the ultrasonic energy to a larger region.

In other embodiments, where the sonodynamic therapy device comprises two or more transducers 904 and the controller 902 may be configured to generate a drive signal to actuate the two or more transducers 904 to produce acoustic ultrasonic pulses where the individual wavefronts, whether converging or diverging, will meet at the same location at the same time to focus the ultrasonic energy. In one embodiment, the controller 902 may adapt the frequency drive for each transducer 904.

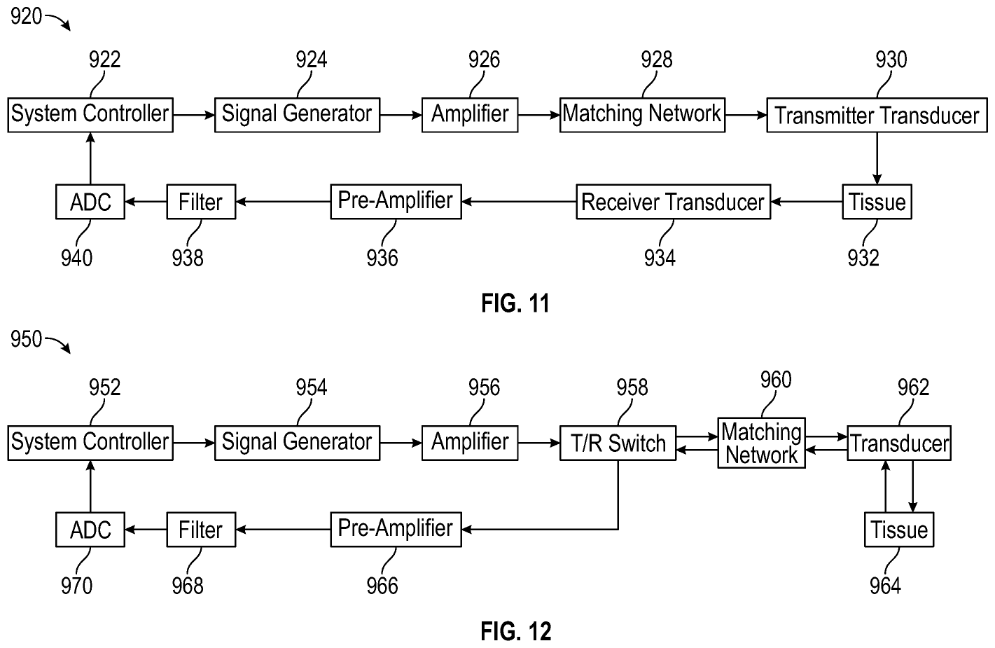

FIG. 11 is a schematic diagram of a sonodynamic therapy system 920 with a separate transmitter transducer 930 and receiver transducer 934, according to at least one embodiment of the present disclosure. The sonodynamic therapy system 920 comprises a system controller 922 to control a signal generator 924 to generate an electrical signal to drive the transmitter transducer 930. The electrical signal is amplified by an amplifier 926 and the drive signal is coupled to the transmitter transducer 930 by a matching network 928 to maximize power transferred to the transmitter transducer 930. The transmitter transducer 930 transmits an acoustic wave into tissue 932 (e.g., lesions) in the treatment region. A receiver transducer 934 detects acoustic waves emitted by the tissue 932. The output of the receiver transducer 934 is a weak electrical signal that is provided to an electronic pre-amplifier 936 that converts the weak electrical signal into an output signal strong enough to be noise-tolerant and strong enough for further processing such as filtering by a filter 938. The output of the filter 938 is provided to an analog-to-digital converter 940 (ADC) that provides a feedback signal to the system controller 922 in digital form. Based on the feedback signal received from the receiver transducer 934 the system controller 922 can adjust the drive signal applied to the transmitter transducer 930. The adjustment may include adjusting the modulation, strength, frequency, phase, or randomization, of the drive signal, or any combinations thereof. The feedback signal may represent tissue depth, tissue thickness, tissue volume, skull thickness, temperature, distance to the treatment region, or a combination thereof.

FIG. 12 is a schematic diagram of a sonodynamic therapy system 950 with a single transmitting and receiving transducer 962, according to at least one embodiment of the present disclosure. The sonodynamic therapy system 950 comprises a system controller 952 to control a signal generator 954 to generate an electrical signal to drive the transducer 962 in transmitter mode. The electrical signal is amplified by an amplifier 956 and is applied to a transmitter/receiver (T/R) switch 958. When the transducer 962 is in transmitter mode, the T/R switch 958 couples the drive signal to the transducer 962 via a matching network 960 to optimize power transferred to the transducer 962. In transmitter mode, the transducer 962 transmits an acoustic wave into tissue 964 (e.g., lesions) in the treatment region. In receiver mode, the transducer 962 detects acoustic waves emitted by the tissue 964. The output of the transducer 962 is a weak electrical signal that is coupled to the T/R switch 958 by the matching network 960. The T/R switch 958 provides the weak electrical signal to an electronic pre-amplifier 966 that converts the weak electrical signal into an output signal strong enough to be noise-tolerant and strong enough for further processing such as filtering by a filter 968. The output of the filter 968 is provided to an ADC 970 that provides a feedback signal to the system controller 952 in digital form. Based on the feedback signal received from the transducer 962 in receiver mode, the system controller 952 can adjust the drive signal applied to the transducer 962 in transmitter mode. The adjustment may include adjusting the modulation, strength, frequency, phase, or randomization, of the drive signal, or any combinations thereof. The feedback signal may represent tissue depth, tissue thickness, skull thickness, temperature, distance to the treatment region, or a combination thereof.

Having described various embodiments of a sonodynamic therapy system 900, 920, 950, 1000, 1100 and components of the sonodynamic therapy system 900, 920, 950, 1000, 1100, the disclosure now turns to a description of the present disclosure that is directed to various embodiments of ensonification drive patterns to create an incoherent field for distributing low intensity energy. In various embodiments, low intensity energy is 20 W/cm² to 0.01 W/cm², including 15 W/cm², 10 W/cm², 8 W/cm², 7.0 W/cm², 6.5 W/cm², 6.0 W/cm², 5.5 W/cm², 0.05 W/cm², 4.5 W/cm², 4.0 W/cm², 3.5 W/cm², 3.0 W/cm², 2.5 W/cm², 2.0 W/cm², 1.5 W/cm², 1.0 W/cm², 0.5 W/cm², 0.4 W/cm², 0.3 W/cm², 0.2 W/cm², 0.1 W/cm², 0.05 W/cm², and any values and ranges therein down to 0.01 W/cm². The ensonification drive patterns may be generated with multiple ultrasonic transducer elements arranged in an array or sub-array structure for generating an incoherent field according to one embodiment. The number of ultrasonic transducer elements and arrangement of the array is a location dependent solution for each disease state. Various embodiments of ultrasonic transducer arrays are described herein.

Having described various embodiments of a sonodynamic therapy system 900, 920, 950, 1000, 1100 and components of the sonodynamic therapy system 900, 920, 950, 1000, 1100, the disclosure now turns to a description of the present disclosure that is directed to various embodiments of ultrasound transducer array geometries, element placement, element shapes, and lens design for activating a sonosensitizer in conjunction with providing sonodynamic therapy. The ultrasound transducer array embodiments described contribute to an incoherent pressure field with a particular energy profile for activating a sonosensitizer. It will be appreciated that the sonodynamic therapy system 900, 920, 950, 1000, 1100 and components thereof may be adapted and configured to drive the ultrasonic transducer arrays described herein.

In another embodiment, provided are ensonification drive patterns that are applied as a pulsed therapy based on the rate limiting step that depletes or reduces local oxygen supply when the sonosensitizer is activated to produce reactive oxygen species. In another embodiment, the present disclosure is directed to ensonification drive patterns that include phase randomization amongst the ultrasonic transducer elements to create the incoherent distributed acoustic field. In another embodiment, the present disclosure is directed to ensonification drive patterns that include element weighting amongst the ultrasonic transducer elements, where select elements are driven at increased or decreased frequency and/or amplitude to create the incoherent distributed acoustic field. In another embodiment, the present disclosure is directed to ensonification drive patterns that also may include frequency, amplitude, and/or phase modulation within each elements pulse to create the incoherent distributed field. In another embodiment, the present disclosure is directed to ensonification drive patterns that also may include inverse apodization or standard apodization techniques across the array or sub-array element patterns to create the incoherent distributed field. In another embodiment, the present disclosure is directed to ensonification drive patterns that also may include alternating drive patterns that utilize only a subset of the elements as a sub-array for adding energy to specific locations in the distributed field. The intensity, amplitude, and frequency of the ensonification drive patterns as well as resultant peak negative pressures are delivered in a range that contributes to a cavitational environment that is safe for healthy tissue within the therapeutic operating field.

Several embodiments provide ensonification drive patterns for activating a sonosensitizer in conjunction with providing sonodynamic therapy. The ensonification drive pattern creates an incoherent field for distributing low intensity energy. In one embodiment, the drive patterns involving multiple ultrasonic transducer elements to be arranged in an array or sub-array structure. The number of ultrasonic transducer elements and arrangement of the array structure is a location dependent solution for each disease state. Due to the spatial element location differences in the array, coherence will only occur at specific location(s) in the therapeutic operating field. As used herein, coherence may be used to describe the properties of the interrelation between the ensonification waves produced by the disease specific array.

Coherence is a measure of a wave's correlation with another wave or another part of the same wave. Temporal coherence is the degree to which a wave can be shifted in time and still correlate well with another wave. Two waves that are continuous, have constant phase differences, and are the same frequency remain correlated even when shifted in time relative to one another. Spatial coherence is the degree to which a wave can be shifted in space and still correlate well with another wave or another part of the same wave. Coherence between two waves may be measured as a spatial difference between the sources of the two waves, as a time difference between the two waves such that one wave is delayed relative to the other wave, or a combination thereof. Two waves may be considered to be coherent when they have a constant relative phase or when they have zero or constant phase difference and the same frequency. By way of example and not limitation, characteristics of coherent sources may include, for example, waves that have a constant phase difference (e.g. are in phase with each other) and have the same frequency. At the same frequency, the phases of the two waves be randomized while maintaining the same phase difference and preventing the phases from combining by constructive or destructive interference. Although the amplitude of the waves does not necessarily contribute to the coherence of the waves, manipulating the amplitude can be used to achieve a more diffuse acoustic field.

FIGS. 8A-12 illustrate various sonodynamic therapy systems 900, 1000, 1100, 920, 950 for generating ensonification drive patterns for sonodynamic therapy. The sonodynamic therapy systems 900, 1000, 1100, 920, 950 can be adapted and configured to drive an array of ultrasonic transducer elements to generate incoherent ensonification drive patterns for activating a sonosensitizer in conjunction with providing sonodynamic therapy.

In various embodiments, cancerous tissue in the lung, breast, colorectal region, prostate and pancreas may be treated using several embodiments described herein using for example, one or more sonosensitizers along with the ultrasound parameters described herein. Tumors that are difficult to access including those surrounded by bony structures are treated in various embodiments, including but not limited to brain or spinal tumors. Treatment of undesired tissue in joints and other orthopedic applications are also provided herein. In some embodiments, sonodynamic therapy is used to improve efficiency of chemotherapeutic molecules, sonoporation, and/or gene delivery. In various embodiments, sonodynamic therapy with an ultrasound array delivering a temporal-average intensity output below 8, 10, 15, 20 W/cm² (e.g., 0.1-8 W/cm², 0.1-4 W/cm², 0.5-5 W/cm² etc.) to cancer tissue can be used to induce and activate sonosensitizer at relative deep depths within a patient's body with or without cavitation, to produce reactive a thermal effect, non-thermal effect, acoustic streaming, radiation forced tissue displacement, an oxygen species, and/or free radicals in a cascade of events that activate the sonosensitizer and in turn damage the cancer cells. In various embodiments, sonodynamic therapy with an ultrasound array delivering a pulse-average intensity output below 8, 10, 15, 20 W/cm² (e.g., 0.1-8 W/cm², 0.1-4 W/cm², 0.5-5 W/cm² etc.) to cancer tissue can be used to induce and activate sonosensitizer at relative deep depths within a patient's body with or without cavitation, to produce reactive a thermal effect, non-thermal effect, acoustic streaming, radiation forced tissue displacement, an oxygen species, and/or free radicals in a cascade of events that activate the sonosensitizer and in turn damage the cancer cells. In various embodiments, sonodynamic therapy can be used with or without photodynamic therapy.

Several embodiments described herein are used synergistically with other cancer therapies, including for example, radiation, chemotherapy, immunotherapy, and cell therapies. In one embodiment, the combination of ultrasound and a sonosensitizer as described herein reduces or eliminates the need for one or more additional complementary treatments. For example, lower doses or fewer additional treatments of chemotherapy, radiation, cell therapy etc. may be needed when cancerous tissue is treated by the combination of ultrasound and a sonosensitizer as described herein, thus enhancing patient care and reducing side effects.

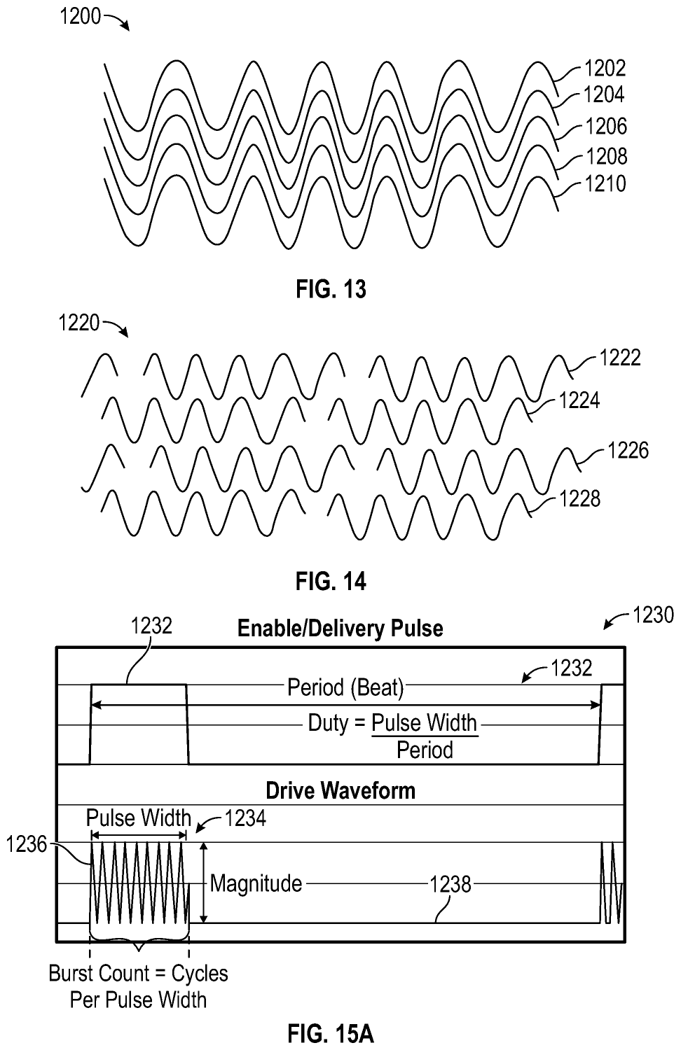

FIG. 13 shows a diagram of a coherent acoustic field 1200 produced by an array of coherent ultrasonic transducer elements in accordance with at least one embodiment of the present disclosure. The coherent acoustic field 1200 comprises, or consists essentially of, a plurality of waveforms 1202, 1204, 1206, 1208, 1210 at the same frequency, phase, and amplitude, for example.

Incoherent sources are opposite of coherent sources. Incoherent sources emit ensonification drive patterns that randomize phase difference across an ultrasonic transducer array. In addition, the frequency and/or amplitude within an ensonification drive pattern also may be modulated to achieve an incoherent source. FIG. 14 shows a diagram of an incoherent acoustic field 1220 produced by an array of incoherent ultrasonic transducer elements in accordance with at least one embodiment of the present disclosure. The incoherent acoustic field 1220 comprises, or consists essentially of, a plurality of waveforms 1222, 1224, 1226, 1228 at different phases relative to each other, for example. As shown in the example of FIG. 14, waveforms 1222-1228 are generated in bursts that are out of phase with other. In some embodiments, the burst frequency is 0.3-3 MHz (e.g., 0.5-1.5, 0.5-1.2, 0.6-1.8, 0.7-1.1, 0.5-2.0 MHz, etc.).

In one embodiment, uniquely emitting incoherent field driving patterns contributes to a cavitational environment for activating the sonosensitizer and promoting sonodynamic therapy. The intensity, amplitude, and frequency of the ensonification drive patterns as well as resultant variable peak negative pressures are additional key contributors to the cavitational environment for activating a sonosensitizer. In one embodiment, uniquely emitting incoherent field driving patterns contributes to an environment for activating the sonosensitizer and promoting sonodynamic therapy without cavitation. The intensity, amplitude, and frequency of the ensonification drive patterns as well as resultant variable peak negative pressures are additional contributors to the environment for activating a sonosensitizer. Also and additionally, incoherent field driving patterns continually shift energy collection points within the therapeutic operating field such that after many cycles of the drive pattern a large treatment volume can gradually be saturated with ultrasonic pressures to broadly activate the sonosensitizer. This ensures that, in one embodiment, extraneous cancer cells in and all around the target treatment site receive therapy. In some instances, anatomic structures may disrupt and/or attenuate the ultrasonic pressures within the preferred treatment region. Where known disruptions may occur in the desired treatment region, the ensonification drive pattern may employ a combination of coherent and incoherent driving patterns to selectively add energy to any weak spots in the therapeutic operating field.

The ensonification drive patterns are applied as a pulsed therapy according to one embodiment. The ensonification drive patterns are applied as a continuous therapy according to one embodiment. Several embodiments herein provide patient safety benefits by applying pulsed drive patterns instead of continuous wave. This avoids the buildup of energy as heat, especially at locations where significant reflections may be occurring. The pulsed drive pattern also dramatically reduces the ability for standing waves to form, thereby mitigating risks associated with continuous wave patterns. The pulsed drive pattern is also important to allowing broad activation of the sonosensitizers in some embodiments. With one embodiment of sonodynamic therapy there is a rate limiting step when the sonosensitizer is activated to produce reactive oxygen species, this process momentarily depletes local oxygen supply. A pulsed drive pattern enables the local oxygen supply to re-saturate, thereby enabling subsequent sonosensitizer activations to occur during subsequent pulses. In one embodiment, continuous wave drive patterns not only introduce a significant increase in potential patient safety hazards, but also could be detrimental to effective administration of sonodynamic therapy as continuous wave drive patterns may not broadly allow for restoration of local oxygen supplies.

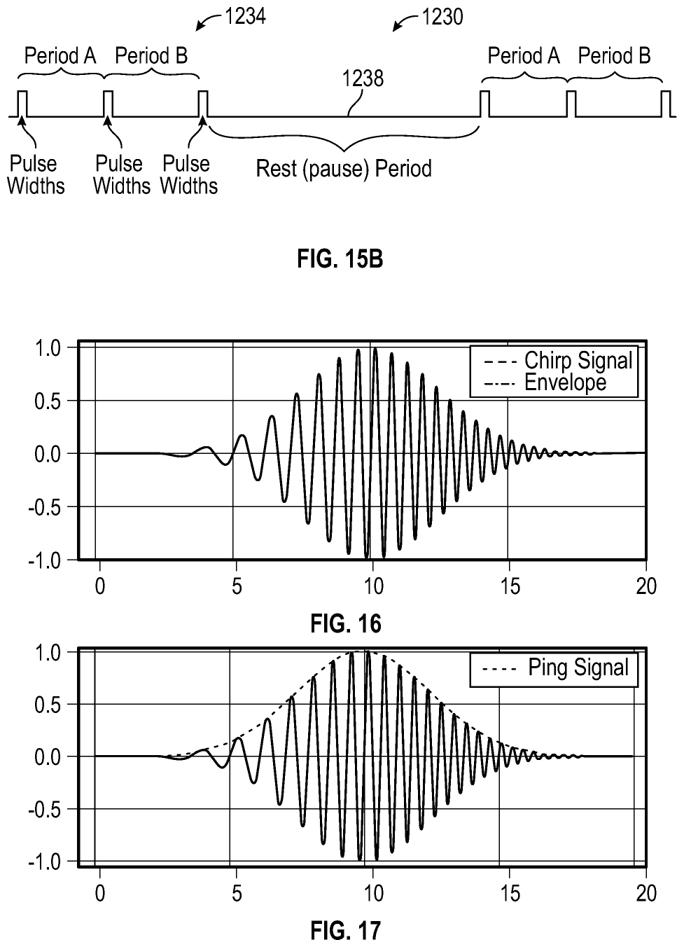

FIG. 15A shows a diagram of a pulsed therapy 1230 in accordance with at least one embodiment of the present disclosure. An enable/delivery pulse 1232 activates a drive waveform 1234 over a period (beat) at a predetermined duty cycle defined as the ratio of the pulse width to the period. The drive waveform 1234 has the same pulse width as the enable/delivery pulse 1232 and repeats over the same period. The drive waveform 1234 also is defined by the magnitude (amplitude) and burst count—the number of cycles of a periodic wave per pulse width 1232. The pulsed nature of the drive pattern for enabling activation of a sonosensitizers can be defined by the pulse width of the pulsed drive waveform 1234 pattern. Each pulse width includes a burst 1236 of drive pattern cycles. In one embodiment, this burst 1236 of drive patterns creates a cavitation environment to activate the sonosensitizers. In one embodiment, this burst 1236 of drive patterns creates a non-cavitational environment to activate the sonosensitizers. Applying a pulsed burst 1236 of drive patterns enables application of relatively higher peak intensity, while still maintain low temporal intensity. In one embodiment, each pulse width includes a burst 1236 of ten to one-thousand drive pattern cycles to create the activation profile of the sonosensitizer. The time 1238 between bursts 1236 accounts for the restoration of local oxygen supplies, and additionally can be manipulated to manage temperature and safety concerns in highly reflective environments.

FIG. 15B shows a diagram of a pulsed therapy 1230 in accordance with at least one embodiment of the present disclosure that includes a pulsed drive waveform 1234 pattern with variable length periods followed by a periodic rest or pause cycle 1238. In various embodiments, a time delay is in a range of 0.1 μs to 100 s, 10 μs to 100 μs, 15 μs to 40 μs, including 0.1 μs, 0.2 μs, 0.3 μs, 0.4 μs, 0.5 μs, 1 μs, 5 μs, 10 μs, 15 μs, 20 μs, 25 μs, 30 μs, 35 μs, 40 μs, 50 μs, 60 μs, 70 μs, 80 μs, 90 μs, 0.1 ms, 0.2 ms, 0.3 ms, 0.4 ms, 0.5 ms, 1 ms, 5 ms, 10 ms, 50 ms, 100 ms, 500 ms, 1 s, 2 s, 3 s, 4 s, 5 s, 6 s, 7 s, 8 s, 9 s, 10 s, 20 s, 30 s, 40 s, 50 s, 60 s, 70 s, 80 s, 90 s, 100 s and any values and ranges therein. In various embodiments, a period rest or pause cycle is in a range of 0.1 μs to 100 s, 10 μs to 100 μs, 15 μs to 40 μs, including 0.1 μs, 0.2 μs, 0.3 μs, 0.4 μs, 0.5 μs, 1 μs, 5 μs, 10 μs, 15 μs, 20 μs, 25 μs, 30 μs, 35 μs, 40 μs, 50 μs, 60 μs, 70 μs, 80 μs, 90 μs, 0.1 ms, 0.2 ms, 0.3 ms, 0.4 ms, 0.5 ms, 1 ms, 5 ms, 10 ms, 50 ms, 100 ms, 500 ms, 1 s, 2 s, 3 s, 4 s, 5 s, 6 s, 7 s, 8 s, 9 s, 10 s, 20 s, 30 s, 40 s, 50 s, 60 s, 70 s, 80 s, 90 s, 100 s and any values and ranges therein. In one embodiment, each pulse has the potential to create cavitation bubbles, and in some instances the cavitation bubbles can accumulate in a cloud that may obstruct or attenuate subsequent pulses. Various combinations of different period lengths as well as rest/pause cycles can be utilized to improve dissipation of the cavitation cloud prior to subsequent pulses. These pulsing parameters also provide additional means to manage and prevent temperature increases in the patient tissues exposed to the ultrasound field.

FIG. 15B illustrates a pulsed therapy in accordance with at least one embodiment of the present disclosure that includes an initial period A followed by a slightly longer period B, followed by a rest or pause period before repeating the pulsing sequence. Additional subsequent periods of continually differing lengths may follow periods A and B. In various embodiments, period lengths and the rest/pause interval may provide additional benefit for activating a sonosensitizer by providing additional time for restoration of local oxygen supplies beneficial for sonodynamic therapy. In one embodiment, the period defines the pulse repetition frequency. In one embodiment, ultrasound transducers create high pitched audible noises when pulsing at a uniform pulse repetition frequency. Such audible noises may not be acceptable in clinical setting while treating patients, especially in a brain cancer application where the transducer is coupled to the head which can amplify the audible noise from the patient's perspective. In one embodiment the period length is randomized from pulse to pulse to reduce audible noise output from the transducer. In various embodiments, the Period A, Period B, and Rest (pause) Period 1238 are in a range of 0.1 μs to 100 s, including 0.1 μs, 0.2 μs, 0.3 μs, 0.4 μs, 0.5 μs, 1 μs, 5 μs, 10 μs, 15 μs, 20 μs, 25 μs, 30 μs, 35 μs, 40 μs, 50 μs, 60 μs, 70 μs, 80 μs, 90 μs, 0.1 ms, 0.2 ms, 0.3 ms, 0.4 ms, 0.5 ms, 1 ms, 5 ms, 10 ms, 50 ms, 100 ms, 500 ms, 1 s, 2 s, 3 s, 4 s, 5 s, 6 s, 7 s, 8 s, 9 s, 10 s, 20 s, 30 s, 40 s, 50 s, 60 s, 70 s, 80 s, 90 s, 100 s and any values and ranges therein. In various embodiments, a period rest or pause cycle 1238 is in a range of 0.1 μs to 100 s, including 0.1 μs, 0.2 μs, 0.3 μs, 0.4 μs, 0.5 μs, 1 μs, 5 μs, 10 μs, 15 μs, 20 μs, 25 μs, 30 μs, 35 μs, 40 μs, 50 μs, 60 μs, 70 μs, 80 μs, 90 μs, 0.1 ms, 0.2 ms, 0.3 ms, 0.4 ms, 0.5 ms, 1 ms, 5 ms, 10 ms, 50 ms, 100 ms, 500 ms, 1 s, 2 s, 3 s, 4 s, 5 s, 6 s, 7 s, 8 s, 9 s, 10 s, 20 s, 30 s, 40 s, 50 s, 60 s, 70 s, 80 s, 90 s, 100 s and any values and ranges therein. In one embodiment, a pulsed therapy is 0.1 μs and a period rest or pause cycle 1238 is 0.1 μs. In one embodiment, a pulsed therapy is 0.1 μs and a period rest or pause cycle is 0.2 μs. In one embodiment, a pulsed therapy is 0.1 μs and a period rest or pause cycle is 0.5 μs. In one embodiment, a pulsed therapy is 1 μs and a period rest or pause cycle is 1 μs. In one embodiment, a pulsed therapy is 1 μs and a period rest or pause cycle is 2 μs. In one embodiment, a pulsed therapy is 1 μs and a period rest or pause cycle is 5 μs. In one embodiment, a pulsed therapy is 1 ms and a period rest or pause cycle is 1 ms. In one embodiment, a pulsed therapy is 1 ms and a period rest or pause cycle is 2 ms. In one embodiment, a pulsed therapy is 1 ms and a period rest or pause cycle is 5 ms. In one embodiment, a pulsed therapy is 1 s and a period rest or pause cycle is 1 s. In one embodiment, a pulsed therapy is 1 s and a period rest or pause cycle is 2 s. In one embodiment, a pulsed therapy is 1 s and a period rest or pause cycle is 5 s.

In one embodiment, a pulsed drive waveform 1234 pattern includes bursts at discrete frequencies, such as in a range of about 20.00 kHz to about 12.00 MHz, including, for example, 20 kHz, 50 kHz, 100 kHz, 250 kHz, 400 kHz, 500 kHz, 550 kHz, 600 kHz, 650 kHz, 700 kHz, 750 kHz, 800 kHz, 850 kHz, 900 kHz, 950 kHz, 1 MHz, 1.1 MHz, 1.2 MHz, 1.3 MHz, 1.4 MHz, 1.5 MHz, 2 MHz, 3 MHz, 4 MHz, 5 MHz, 10 MHz, 12 MHz, and any values and ranges therein, such 0.5 to 1.5 MHz, 0.6 to 1.4 MHz, 0.7 to 1.1 MHz, 0.8 to 1.2 MHz, 1 to 5 MHz, etc. More particularly, the transducers 150, 400, 450 may be driven at ultrasonic frequencies in a range of about 650.00 kHz to about 2.00 MHz. In one embodiment, the transducers 150, 400, 450 may be driven at ultrasonic frequencies in a range of about 500 kHz to about 1.3 MHz, about 700 kHz to about 1.1 MHz, 900.00 kHz to about 1.20 MHz, 975 kHz-1.1 MHz, and as examples, in one embodiment, at about 1 MHz, 1.03 MHz, 1.06 MHz, 1.10 MHz, 1.20 MHz, etc.) In one embodiment, a pulsed drive waveform 1234 pattern includes a burst at 0.1 MHz, 0.2 MHz, 0.4 MHz, 0.5 MHz, 0.6 MHz, 0.7 MHz, 0.8 MHz, 0.9 MHz, 1.0 MHz, 1.1 MHz, 1.2 MHz, 1.3 MHz, 1.4 MHz, 1.5 MHz, 1.6 MHz, 1.7 MHz, 1.8 MHz, 1.9 MHz, 2.0 MHz, 2.5 MHz, 3.0 MHz, 3.5 MHz, 4.0 MHz, 4.5 MHz, 5.0 MHz, 6 MHz, 7 MHz, 8 MHz, 9 MHz, 10 MHz and any values or ranges therein. In one embodiment, a pulsed drive waveform 1234 pattern includes one, two, three or more discrete bursts at 0.1 MHz, 0.2 MHz, 0.4 MHz, 0.5 MHz, 0.6 MHz, 0.7 MHz, 0.8 MHz, 0.9 MHz, 1.0 MHz, 1.1 MHz, 1.2 MHz, 1.3 MHz, 1.4 MHz, 1.5 MHz, 1.6 MHz, 1.7 MHz, 1.8 MHz, 1.9 MHz, 2.0 MHz, 2.5 MHz, 3.0 MHz, 3.5 MHz, 4.0 MHz, 4.5 MHz, 5.0 MHz, 6 MHz, 7 MHz, 8 MHz, 9 MHz, 10 MHz and any values or ranges therein, and including random bursts, sequential advancing bursts, sequential declining bursts, skipping bursts, and other burst patterns. In various embodiments, a repeating signal may be pulse-width modulated, duty-cycle modulated, phase modulated, frequency modulated, randomized phase modulated, or may be modulated using any suitable modulation technique to produce a desired acoustic pulse packet. In one embodiment, all elements in an array fire simultaneously. In one embodiment, all elements in an array fire sequentially. In one embodiment, all elements in an array fire randomly. In one embodiment, all elements in an array fire incrementally, such as at 0.5, 0.6, 0.7, 0.8, 0.9, 1.0, 1.1, 1.2, 1.3, 1.4, 1.5, 1.6, 1.7, 1.8, 1.9, 2.0 MHz and any values therein. In one embodiment, a reverse incremental sequence is used. In one embodiment, a lower frequency results in less cavitation. In one embodiment, a lower frequency provides for a greater potential to collapse bubbles. In one embodiment, a higher frequency provides for a greater potential for more cavitation.

The ensonification drive patterns include phase randomization amongst ultrasonic transducer elements according to one embodiment. The phase differences amongst the waves generated are randomized. This drive pattern provides an important embodiment in creating the incoherent distributed therapeutic operating field. The randomization technique may utilize a normal distribution, or in other embodiments it may be advantageous to pull from various random distributions to setup random phases. In one embodiment, the method comprises selecting the phase of each element across the array in a randomized manner between 0-220 degrees (e.g., 0-45, 0-90, 1-135, 0-180, 0-200, 45-90, 45-135, 45-180, 45-220, 90-135, 90-180, 90-220, 120-220, 120-180, 120-150, 180-220, and/or 200-220 degrees of the phase, followed by dispersion adjustments to select groups of elements for the remaining 140-360 degrees of the phase (e.g., 140-300, 140-270, 140-225, 140-180, 140-150 degrees.

The ensonification drive patterns may include frequency modulation within each elements pulse to enhance the incoherent distributed field according to one embodiment. Varying the frequency within the burst of wave packets blurs the wave fronts, thereby providing a more homogenous therapeutic operating field. Broadly and evenly spreading the energy is necessary for robust activation of the sonosensitizers.

The ensonification drive patterns may include inverse and standard apodization profiles across the array elements, as well as a flat apodization profile according to one embodiment. Temporal apodization profiles within a drive pattern cycle are also a means that may be utilized to enhance the incoherent distributed field. Apodization is usually an ultrasound imaging technique that involves varying the amplitude across the aperture of the ultrasonic transducer, such that the transducer elements at the center of the probe head are electrically excited with a voltage of greater amplitude to those at the edges. Ultrasound imaging apodization seeks to reduce the amplitude of side lobes for better overall image resolution. An inverse of this drive pattern can be uniquely applied for directing energy into a therapeutic operating field for activating a sonosensitizer according to one embodiment. The unique inverse apodization for sonodynamic therapy provides greater energy to the ultrasonic transducer elements at the outer edges of the array compared to those near the center of the array axis. For this reason, inverse apodization both broadens the beam width, as well as results in deeper ensonification regions. The excitation scheme may involve smooth and/or discrete steps that help collect and distribute the energy across the therapeutic operating field. In the context of this therapeutic device, array or sub-array based apodization may be utilized to focus energy in a smaller therapeutic operating field. This can be helpful when optimizing the therapeutic operating field in response to varying skull thicknesses, for example.

The ensonification drive patterns include alternating drive patterns according to one embodiment. Furthermore, some of the alternating drive patterns utilize only a subset of the elements as a sub-array for adding energy to specific locations in the distributed field. This is achieved, for example, through a selective process of coherently selecting the elements with directivity to the location of interest then providing phase randomization across those sub-array elements in order to have a field as incoherent as possible in the location of interest. The pulsed nature of the preferred drive patterns for sonodynamic therapy in one embodiment is disclosed herein. Each pulse could include the same burst of drive patterns, or additionally some alternating frequency of pulses could provide alternative drive patterns. The alternative drive patterns provide means to further saturate the therapeutic operating field with preferred waveform characteristics for activating a sonosensitizer in one embodiment. The alternating drive patterns may use all the ultrasonic transducer elements in the array, while other alternating drive patterns may use only a subset of the transducer elements in the array as a sub-array. Alternating drive patterns within a sub-array enables energy to be added to specific weak spots in the therapeutic operating field, without employing any type of coherent focused drive pattern.

FIGS. 16-21 illustrate various embodiments of ultrasonic transducer arrays and techniques for packing elements of the ultrasonic transducer arrays according to various embodiments. The ultrasonic transducer arrays may be driven by the sonodynamic therapy systems 900, 1000, 1100, 920, 950. The sonodynamic therapy systems 900, 1000, 1100, 920, 950 may be adapted and configured to drive the ultrasonic transducer arrays described herein to generate ensonification drive patterns for sonodynamic therapy. An ultrasonic transducer is a device that is capable of generating and receiving ultrasonic vibrations according to one embodiment. An ultrasonic transducer comprises an active element. The active element is a piezoelectric or single crystal material which converts electrical energy to ultrasonic energy. Various embodiments of ultrasound array geometries for sonodynamic therapy may include large apertures, that contour with and/or are close fitting to the body according to one embodiment. Large apertures are defined as those that are the same size or larger than the lesion being treated. The aspect ratio of the aperture to lesion size enables initiation of a broad incoherent field to ensure the lesion and surrounding tissue receive therapy. In one embodiment, the array is close fitting with the body. For example, in a brain cancer embodiment the array could be a close-fitting helmet, or even individual elements placed in an array pattern directly on the head. In one embodiment, ultrasound modifies the blood brain barrier (BBB). In one embodiment, ultrasound facilitates delivery of a drug and/or sonosensitizer across the blood brain barrier.

In various embodiments, the ultrasonic transducer elements that make up an array may be configured in a linear array, rectangular array, circular array, concentric circular array, spiral array, Archimedean spiral array, sunflower spiral array, curved array, or any combination thereof, or sparse variations thereof, as described herein according to various embodiments.

In one embodiment, an array comprising multiple active ultrasonic transducer elements is randomly arranged and irregularly placed in a non-uniform distribution in accordance with at least one embodiment of the present disclosure. In one embodiment, the diameter of an array is about 150 mm, without limitation, and the diameter of the ultrasonic transducer elements can be selected in the range of 0.5 mm to 20 mm, without limitation, depending on the frequency of the excitation signal and the speed of sound in water. It will be appreciated that some of the ultrasonic transducer elements may be deactivated.

In various embodiments, ultrasonic transducer element shapes include circular or disc shapes and concentric elements, for example. In one embodiment, an array of active ultrasonic transducer elements is arranged in concentric circles with additional active ultrasonic transducer elements disposed on outer portion of the outer ring according in accordance with at least one embodiment of the present disclosure. In various embodiments, ultrasonic transducer element shapes include circular, square, rectangular, or disc shapes and a plurality of elements, for example. In one embodiment, an array of active ultrasonic transducer elements is arranged with additional active ultrasonic transducer elements disposed on an outer portion of the device according in accordance with at least one embodiment of the present disclosure. In one embodiment, the diameter of the array is about 150 mm, without limitation, and the diameter of the ultrasonic transducer elements can be selected in the range of 0.5 mm to 20 mm, without limitation, depending on the frequency of the excitation signal and the speed of sound in water.

In one embodiment, an array of ultrasonic transducer elements is arranged concentrically in accordance with at least one embodiment of the present disclosure. In one embodiment, an array of ultrasonic transducer elements is arranged along a curved surface in accordance with at least one embodiment of the present disclosure. In various embodiments, the diameter of the overall array is in a range of about 100 mm-200 mm, including but not limited to 100 mm, 125 mm, 150 mm, 165 mm, 175 mm, 200 mm and any values therein without limitation, and the diameter of the ultrasonic transducer elements can be selected in the range of 0.5 mm to 20 mm, including 0.5 mm, 1 mm, 3 mm, 5 mm, 7 mm, 10 mm, 12 mm, 15 mm, 18 mm, and 20 mm without limitation, depending on the frequency of the excitation signal and the speed of sound in water. In various embodiments, the diameter of the ultrasonic transducer elements may be same or may be different with the diameter of the transducer elements selected within the ranges set forth in this disclosure. It will be appreciated that some of the ultrasonic transducer elements may be activated or deactivated collectively, in groups, or individually. In various embodiments, the array comprises 1 to 1024 ultrasonic transducer elements, including 1, 2, 4, 8, 16, 32, 64, 128, 256, 384, 512, 640, 678, 896, 1024 elements. In various embodiments the array comprises 100, 200, 300, 400, 500, 600, 700, 800, 900, or 1000 ultrasonic transducer elements. In one embodiment, the array has 256 transducer elements that each have a 5 mm diameter. In one embodiment, the spacing between adjacent elements is constant. In one embodiment, spacing between adjacent elements incrementally increases from the center of the array outward toward an outer diameter of the array, wherein the spacing between adjacent valences and/or concentric rings of elements incrementally increases from the center of the array toward an outer diameter or circumference of the array. In one embodiment, spacing between adjacent elements incrementally decreases from the center of the array outward toward an outer diameter of the array, wherein the spacing between adjacent valences and/or concentric rings of elements incrementally decreases from the center of the array toward an outer diameter or circumference of the array. In one embodiment, spacing between adjacent elements incrementally increases from the center of the array outward toward an outer distance of the array, wherein the spacing between adjacent valences and/or rings of elements incrementally increases from the center of the array toward an outer distance of the array. In one embodiment, spacing between adjacent elements incrementally decreases from the center of the array outward toward an outer dimension of the array, wherein the spacing between adjacent valences and/or rings of elements incrementally decreases from the center of the array toward an outer dimension of the array.

Having described various embodiments of a sonodynamic therapy system 900, 920, 950, 1000, 1100 and components of the sonodynamic therapy system 900, 920, 950, 1000, 1100, the disclosure now turns to a description of the present disclosure that is directed to various embodiments of apparatuses, systems, and methods for selectively locating and holding an ultrasonic transducer array in preferred locations for treatment, coupling that array to a patient's body for efficient and safe transfer of energy, as well as optimization routines that take into account, and compensate as necessary for, variations in transmission through the body (e.g., intervening tissue) to enable a therapeutic operating field with appropriate energy profile for activating a sonosensitizer.

In various embodiments, a wearable receptacle referred to as a patient interface is placed over/on and fitted to the patient's head or other body part. The patient interface provides location registration between a patient specific anatomy and an ultrasonic transducer array detachably coupled to or integrated with the patient interface, which in turn guides placement and location of the therapeutic operating field for providing sonodynamic therapy.

In various embodiments, the patient interface 180 includes one or more alignment and/or orientation features establish a true location registration. In various embodiments, an alignment device 850, 1900 is used in conjunction with the ultrasound transducer system 100 to help align the treatment with a treatment site of the patient. In one embodiment, alignment of positions or placements is manual (e.g., by the system operator). In one embodiment, alignment of positions or placements is automated via robot arm or other mechanism (e.g., tracks, wheels, bearings, gears, rails, motors, actuators, hydraulics, pneumatics, magnetism, etc.) for linear, rotational, arcuate, curved, or other actuation.

The alignment and/or orientation features are shaped and/or sized to interface with and receive bony landmarks on the head such as the zygomatic arch, mastoid process, mastoid tip of temporal bone, lateral eye, and middle arch of eyebrows to establish a true location registration for providing sonodynamic therapy. In one embodiment, the patient interface includes alignment and/or orientation features that are shaped and sized to receive at least two anatomical features on a patient's head. In one embodiment a targeting template is placed on the patient to facilitate alignment of the transducer to the various treatment sites. In various embodiments, the targeting template is a wearable elastic template with markers to facilitate treatment, such as by demarking a grid, positions based on anatomy, or marking of the skin with indicators. In one embodiment, the targeting template is a cap. In one embodiment, the targeting template is a band configured to wrap around a head, neck, chest, torso, back, waist, leg, buttock, genital area or other body part. In one embodiment, the targeting template is drawn on the body. In one embodiment, the targeting template includes measurement gradients that allow the user to customize treatment locations to patient specific anatomical size. In some embodiments, the targeting template remains in place during ultrasound treatment. In some embodiments, the targeting template is made to be removable prior to ultrasound treatment.

Once the patient interface is properly aligned and placed on the patient, it can be effectively fixed in place by straps, adhesive, tape, or any other suitable fixtures that tightly secure the patient interface to the patient. In certain embodiments, the patient interface is coupled to a robotic arm that can perform minor and/or major adjustments to the position of the patient interface with respect to the patient's head. In at least one example, the robotic arm is decoupled from the patient interface once it is secured to the patient's head in a treatment position.

In various embodiments, the patient interface provides a receptacle to receive an ultrasound probe configured for sonodynamic therapy. Therefore, the ultrasound probe treatment location is established by the patient interface device. This location ultimately determines the placement of the therapeutic operating field for providing sonodynamic therapy. The patient interface may alternatively include multiple receptacles for receiving multiple ultrasound probes and/or for discreetly moving a single probe through multiple defined treatment locations. Preferred treatment locations may be a fixed predetermined pattern, or alternatively maybe customized based on specific disease location for each patient.

In certain embodiments, the patient interface includes structures and/or features that guide placement of an ultrasonic transducer array into a position for activating a sonosensitizer. In certain embodiments, the patient interface includes multiple structures or features that guide placement of multiple transducer arrays into positions for activating a sonosensitizer.

In other embodiments, the patient interface provides a progression of discrete steps utilizing a single array serially across the preferred treatment positions. In certain embodiments, the patient interface includes an array holder, which can be adjusted, automatically or manually, to move the array into preferred treatment locations. Preferred treatment locations for the array, in several embodiments, may include areas on the skull that are more conducive to acoustical coupling based on geometry, anatomy, and/or preferred anatomical attenuation. Preferred treatment locations may also be indexed to or correlated with a camera, CT, MRI, or other imaging data that provides known anatomical inputs to guide placement and/or therapy parameters for the array. Furthermore, the preferred treatment location, in one embodiment, takes into account the diseased location as an input, thereby placing the array in a position that ensures ultrasonic energy is directed to the diseased region and surrounding tissue.

In various embodiments, a controller such as, for example, the controller 902 may receive imaging data such as, for example, a camera, CT, MRI or other imaging data that provides known anatomical inputs to guide placement and/or therapy parameters for the array. Based on the imaging data, the controller 902 may select a treatment location for the ultrasonic transducer array. In certain embodiments, the array holder is operably coupled to a motor. In such embodiments, the controller 902 may cause the motor to move the array holder relative to the patient interface to a selected treatment location. In various embodiments, the controller 902 may cause the user interface 1004 to communicate a selected treatment location to a user.

In another embodiments, the ultrasonic transducer array is integrated with the patient interface, such that placing and locating the patient interface, is also placing and locating the array.

Once the array is placed in the proper position(s) for treatment, it is properly coupled to the patient for transmission according to one embodiment. In some embodiments for treatment on the head, hair will be removed so as not to interfere with acoustic coupling. An acoustically conductive gel is common in the industry. In various embodiments, an acoustic coupling membrane is attached over the exit plane of the ultrasonic transducer array. The acoustic coupling membrane can be selectively inflated and deflated to further guide placement and location of the ultrasonic transducer array, further guiding placement of the therapeutic operating field. In various embodiments, the acoustic coupling membrane comprises an elastic material with acoustically neutral properties so as to provide minimal ultrasound attenuation.

In various embodiments, the acoustic coupling membrane defines a cavity with the patient interface. The ultrasonic transducer array may project from the patient interface toward the cavity. An acoustical coupling agent such as, for example, degassed water can be utilized to fill the cavity to a predetermined volume. The volume of the acoustical coupling agent contained within the membrane can be selectively adjusted to reposition the location of the ultrasound array. Selectively controlling position of the array with the coupling membrane enables selective guidance of the therapeutic operating field. The compliance in the membrane allows it to conform to patient's anatomy at the treatment location for acoustical coupling.

Any suitable valve can be utilized to insert and/or remove the acoustical coupling agent into the cavity to inflate and/or deflate the acoustic coupling membrane according to one embodiment. One or more sensors such as, for example, pressure sensors can be employed by the controller 902 to assess the volume of the acoustical coupling agent in the cavity.

In certain embodiments, the volume of the acoustic coupling agent can be selectively adjusted to reposition the location of the ultrasonic transducer array. Selectively controlling position of the array with the coupling membrane enables selective guidance of the therapeutic operating field according to one embodiment. In certain embodiments, the inflation and/or deflation of the acoustic coupling membrane can be used in concert with the array holder to control the location of the array and distance away from the skull. This distance can be discretely adjusted or dynamically adjusted to vary the therapeutic operating field position and penetration depth during treatment according to one embodiment.

In certain embodiments, the acoustic coupling agent is circulated to remove residual heat from the therapeutic operating field during treatment. In certain embodiments, the acoustic coupling agent is also chilled to remove residual heat from the therapeutic operating field during treatment. In certain embodiments, the temperature of the acoustic coupling agent is monitored as a safety provision. For example, the patient interface can include one or more temperature sensors 810, 1118 according to one embodiment. In one example, as described elsewhere herein, a processing unit 1104 is coupled to temperature sensors 810, 1118 and receives patient temperature feedback through the ADC 1010. The processing unit 1104 controls a cooling system 1120 based at least in part on the patient temperature feedback signal.

Transcranial ultrasound delivery has many challenges. The skull acts as a strong reflector, as well as scatterer and absorber of ultrasound energy. There is also a known large patient to patient variation in skull attenuating characteristics. Patient specific information is desirable that takes into account variations in transmission through the patient's skull to optimize a therapeutic operating field for activating a sonosensitizer. The patient specific information could be an input from a camera, CT or MRI or other image file that includes skull thickness data by location. The output of the individual ultrasound elements, and/or the entire array collectively, and/or subsections of the array could be adjusted based on inputs from the camera, CT, MRI, or other image file. Additionally, or alternatively, a sonodynamic therapy system itself (e.g. system 900, described in greater detail in connection with FIGS. 9, 10) could be used to collect patient specific transcranial transmission data for calibrating the optimal ultrasound array output(s).

In various examples, a sonodynamic therapy system (e.g. system 900, described in greater detail in connection with FIGS. 9, 10) includes one or more transcranial optimization routines for calibrating the ultrasonic transducer array 904 to patient specific attributes to establish appropriate ultrasound ensonification parameters according to one embodiment. The controller 902 may be configured to execute one or more control algorithms to calibrate the ultrasonic transducer array 904 to patient specific attributes such as, for example, a skull thickness according to one embodiment. Furthermore, the controller may be configured to cause the ultrasound transducer array 904 to activate a sonosensitizer in a treatment region in the anatomical structure per ultrasound ensonification parameters established by calibrating the ultrasound transducer array to the patient specific attributes, In one embodiment, a patient specific attribute is anatomical. In one embodiment, a patient specific attribute is non-anatomical.

In certain examples, the controller 902 may determine whether skull thickness measurements are within the acceptable nominal range according to one embodiment. In one embodiment, a digital imaging and communications (DICOM) image from a camera, computerized tomography (CT), magnetic resonance imaging (MRI) or other imaging source could be an input to the device controller 902. The imaging data can be analyzed by the controller 902 to determine whether skull thickness measurements are within the acceptable nominal range. Accordingly, the controller 902 may employ imaging data such as, for example, a CT scan as a screening tool, whereby only patients whose skull thickness measurements fall within a nominal or prescribed range are indicated for treatment. Imaging data indicative of a skull thickness could also be utilized to optimize frequency, and array location for most beneficial therapeutic operating field.

Additionally, or alternatively, the controller 902 may cause the ultrasonic transducer array 904 to generate pulses to interrogate the skull at several different frequencies in a calibration algorithm according to one embodiment. The controller 904 then assesses the percent of reflected energy at the various frequencies. In some cases, the frequency with lowest relative reflected energy correlates with the frequency that has highest transmitted energy through the skull. In at least one example, the calibration process involves measuring distances to the skull using time of flight (with short pulses). It may also be possible to measure the inclination of the skull relative various element. If the skull is too inclined relative to nearby elements, in one embodiment, the controller 902 is configured to limit the energy going to those elements.

As described above in greater detail, it can be possible to take measurements or get a rough image of the skull 510 as shown in FIG. 3B according to one embodiment. This can be facilitated if the transducers 150 are fixed to a rigid shell and their relative positions and orientations are known. Rough measurements can be used to adjust the treatment algorithm by measured parameters such as skull thickness, “t,” or skull density, “ρ.” Each transducer 150 may send out an acoustic pulse and listen for an echo. The echoes can be used for a quick estimate of the skull thickness, “t,” or skull density, “ρ,” under each transducer 150. For treatment of tumors in other body parts of the patient, the sonodynamic therapy system 900 may be adapted and configured to couple to the body of the patient.

Further to the above, the calibration process may include a check that the probe is adequately coupled to the patient by air/bubble ultrasound detection techniques according to one embodiment.

In various embodiments, one or more drive signals, which can be executed by the controller 902, include a chirp signal, which can be similar to a sinusoid with a continuously varying frequency. An example of a chirp signal is illustrated in FIG. 16. Multiple overlapping echoes of a chirp signal can be separated in time. Because a sinusoid is identical with respect to shifts in 1 cycle, it cannot be easily separated out in time. In other words, the auto-correlation of a sinusoid has periodic peaks in time spaced 1 cycle apart. The varying frequency of the chirp signal causes the peaks and valleys of a chirp signal to only line up with itself in one way such that the autocorrelation has a single peak. In various embodiments, the chirp signal can be a longer signal in time than a short “ping,” so more energy can be used as an input to perform the calibration. In certain examples, the chirp signals are shaped with an envelope function to have a gently increasing and decreasing peaks according to one embodiment. An example enveloped chirp signal is illustrated in FIG. 17. A chirp signal with a rectangular envelope has abrupt changes. The abrupt changes in the echo that is returned from a rectangular envelope chirp could be from the input signal or from the thing being imaged. The received chirp signal can be integrated in the frequency domain to investigate the transmission across multiple frequencies with one integration. Furthermore, the received chirp signal can be convolved with the time reversed of the transmitted chirp signal to accurately calculate the skull boundary.

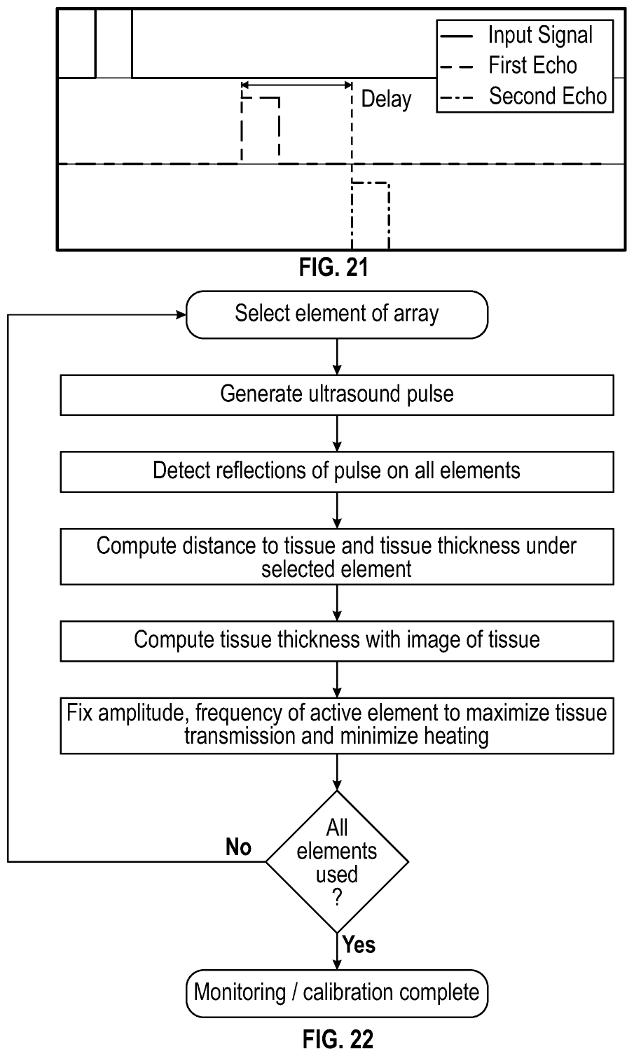

Additionally, or alternatively, an impulse input signal can be utilized in the calibration process according to one embodiment. The impulse input signal may include a sharply increasing and decreasing pulse. An example impulse input signal is illustrated in FIG. 20. This short signal facilitates echoes resolution in time. Typically, an impulse does not contain significant energy—it's limited by the duration and peak pressure. Therefore, a series of impulses separated by x nsec can be utilized for an application intended for an integrated temporal energy. FIG. 21 illustrates an impulse input signals and the resulting echoes. Other input signals might be used. The delay between the two echoes is indicative of a skull thickness. The delay from the impulse input signal to the first echo signal is indicative of the distance to the skull surface.

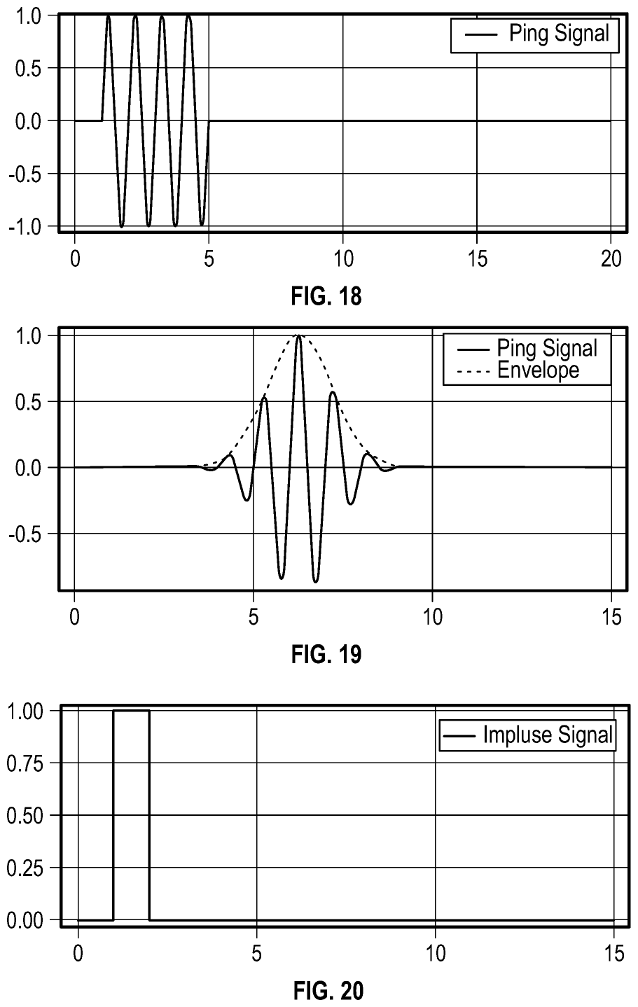

In various embodiments, a ping signal may be used to drive the ultrasound device. In one embodiment, the ping signal is a square signal. Additionally, or alternatively, a ping input signal can be utilized in the calibration process according to one embodiment. A ping is defined by a short burst of pulses at a particular frequency. An example of a square ping signal and a smooth ping signal are illustrated in FIG. 18 and FIG. 19, respectively. A ping input signal typically includes a lesser frequency content than a chirp input signal. Because the input pulse correlates with itself at several locations, the returning echoes are more difficult to distinguish. In various embodiments, an element by element frequency altered pulse burst can be utilized to drive the ultrasound device with one or more ping signals, such as one or more square signals. Additionally, or alternatively, an element by element frequency altered pulse bursts can be utilized in the calibration process. This may include outer elements utilizing lower frequency than inner elements. In the case where outer array elements are farther from the desired therapeutic operating field this can improve the energy available from the outer elements. Additionally, the element frequency is optimized for skull penetration either directly related to skull thickness or alternately, frequency-dependent transmission efficiency. In various embodiments, randomized local frequency content similar to the chirp, but temporally overlapping frequencies transmitted into the tissue for treatment could be utilized with the ultrasound device. Additionally, randomized local frequency content similar to the chirp, but temporally overlapping frequencies transmitted into the brain could be utilized in the calibration process. In one embodiment, randomization could be spread over a localized sub-array (nearest neighbor elements), in another embodiment frequency randomization over the entire array could be deployed in order to optimize the therapeutic operating field.

Further to the above, in various examples, the calibration process will include combinations of one or more of the chirp, ping, and/or impulse input signals at various suitable frequencies and/or amplitudes according to one embodiment. In at least one example, short pings are utilized to interrogate the skull at several different frequencies and/or amplitudes. This overcomes one of the downsides of a short ping, the low frequency content. Additionally, or alternatively, Amplitude modulated pulse bursts can be utilized. Along the length of the pulse burst, the amplitude could change. This would have the effect of focusing deeper or shallower or moving energy to a different part of the therapeutic operating field when applied on a sub-array basis to target specific areas of the brain based on a prior knowledge of skull topology and thickness, for example.

In various examples, the calibration process assesses the position of a target tissue such as, for example, a tumor within an anatomical structure in contact with the patient interface of the sonodynamic therapy system according to one embodiment. For example, the controller 904 may utilize external imaging data and/or ultrasonic imaging data collected by the sonodynamic therapy system itself. The controller 904 may adjust the output of various elements of the ultrasonic transducer array based on the relative position of the individual elements with respect to the target tissue. In at least one example, the outer elements, which are further away from the target tissue, are adjusted to a lower frequency than inner elements that are closed to the target tissue. Additionally, or alternatively, the calibration process optimizes the output of the elements for skull penetration either directly related to localized skull thickness or alternately, frequency-dependent transmission efficiency.

Furthermore, in certain examples, the calibration process employs randomized local frequency content similar to the chirp, but temporally overlapping frequencies transmitted into the brain according to one embodiment. In various embodiments, randomization could be spread over a localized sub-array that can include the nearest or neighboring elements. In another example, the frequency randomization over the entire array could be deployed in order to optimize the therapeutic operating field.

The controller 902, employing one or more of the described optimization techniques of the calibration process, may determine the distance to a skull surface of a patient (e.g. skull 510 as shown in FIG. 3C) wearing the sonodynamic therapy system 900 according to one embodiment. In one example, the time of flight is employed to estimate the distance from transducers 150 to the skull 510. Furthermore, the controller 902 can also estimate the thickness of the skull based on echoes received from the outer and inner skull surfaces. The differences between the time of flight of these two echoes can inform the approximate skull thickness.

Within the transducer frequency bandwidth, it is likely that some frequencies would have lower reflected energy characteristics and correspondingly better skull transmission characteristics which could be beneficial for optimizing the therapeutic operating field according to one embodiment. In particular, one frequency may result in lower reflected energy. In some cases, this frequency with lowest relative reflected energy correlates with the frequency that has highest transmitted energy through the skull. Accordingly, the controller 902, employing one or more of the described optimization techniques of the calibration process, may interrogate the skull 510 at different frequencies, and compare the energy reflected by the skull for each of the frequencies to determine frequencies with the highest skull transmission to maximize the amount of energy transmitted though the skull. Furthermore, in certain examples, the controller 902, employing one or more of the described optimization techniques of the calibration process, may maximize the size of the therapeutic operating field through changes in pulses or system components for treating brain cancer.

In some embodiments, sonodynamic therapy is used to improve efficiency of sonoporation, gene therapy, and/or chemotherapeutic treatments. In various embodiments, sonodynamic therapy is used to activate a sonosensitizer within a patient's body, or on the surface of the patient's body. In various embodiments, sonodynamic therapy can be used with or without photodynamic therapy. Several embodiments described herein are used synergistically with other therapies, including for example, radiation, chemotherapy, immunotherapy, and cell therapies.

FIG. 22 is a logic flow diagram of a process depicting a control program or a logic configuration for monitoring an ultrasonic transducer array of a sonodynamic treatment system (e.g. system 900), in accordance with at least one embodiment of the present disclosure. The monitoring process of FIG. 22 includes selecting an element of the ultrasonic transducer array 904, generating an ultrasound pulse with this element, and detecting reflections of the pulse on all elements of the ultrasonic transducer array 904. The calibration process of FIG. 22 further includes computing a minimum distance from the one of the plurality of elements to the skull, wherein the minimum distance is a distance from the one of the plurality of elements to a skull portion adjacent, or under, the one of the plurality of elements, and wherein the controller is configured to compute a skull thickness at the skull portion. The calibration process of FIG. 22 further includes comparing the computed skull sickness with imaging data of the patient's skull such as, for example, CT scans. The calibration process of FIG. 22 further includes setting, or fixing, amplitude and frequency of the active element to maximize an ultrasound transmission rate, or efficiency, through the skull. Additionally, or alternatively, the calibration process of FIG. 22 further includes fixing the amplitude and frequency of the active element to minimize skull heating during a sonodynamic treatment performed by the system 900. In certain embodiments, the calibration process of FIG. 22 is repeated until all, or at least a predetermined subset, of the elements of ultrasonic transducer array 904 are calibrated to maximize skull transmission and/or minimize skull heating.

Maximization of skull transmission and/or minimization of skull heating are assessed based predetermined thresholds according to one embodiment. For example, acceptable values for a maximized skull transmission are values equal to or greater than a predetermined threshold indicative of skull transmission. Likewise, acceptable values for a minimized skull heating are values equal to or less than a predetermined threshold indicative of skull heating.

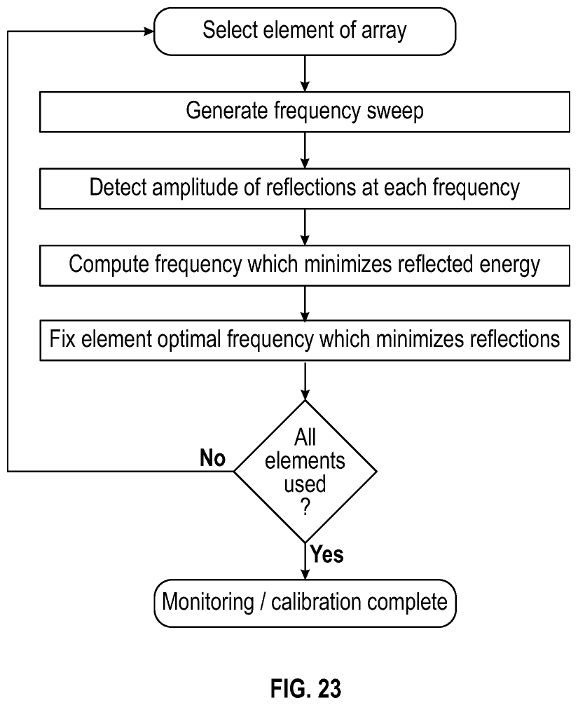

FIG. 23 is a logic flow diagram of a process depicting a control program or a logic configuration for calibrating an ultrasonic transducer array of a sonodynamic treatment system, in accordance with at least one embodiment of the present disclosure. The calibration process of FIG. 23 includes selecting an element of the ultrasonic transducer array 904, generating a frequency sweep with this element, and detecting Amplitude of energy reflected at each frequency of the frequency sweep. The calibration process of FIG. 23 further includes computing an optimal frequency for the element, wherein the optimal frequency is one that minimizes the energy reflected beyond a predetermined threshold. The calibration process of FIG. 23 further includes setting the element to the optimal frequency. In certain embodiments, the calibration process of FIG. 23 is repeated until all, or at least a predetermined subset, of the elements of ultrasonic transducer array 904 are calibrated to optimal frequencies.

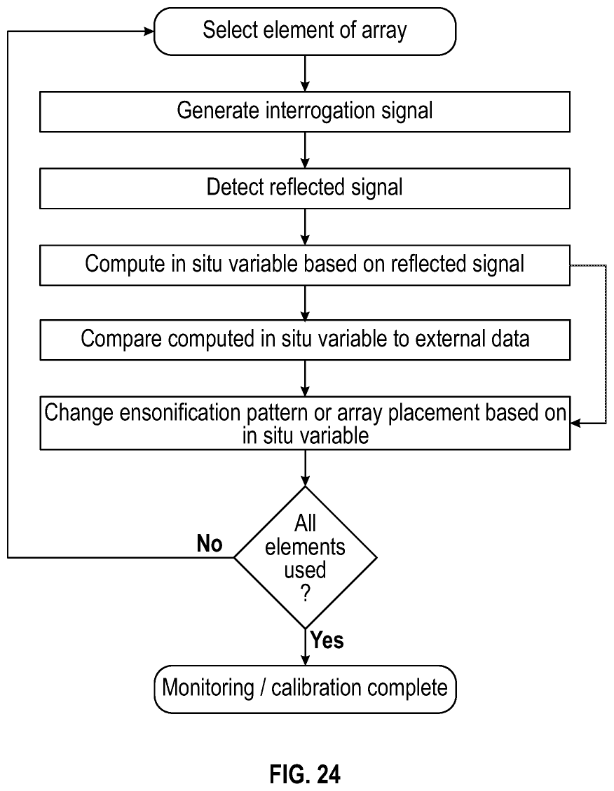

FIG. 24 is a logic flow diagram of a process depicting a control program or a logic configuration for calibrating an ultrasonic transducer array of a sonodynamic treatment system, in accordance with at least one embodiment of the present disclosure. The calibration process of FIG. 24 includes selecting an element of the ultrasonic transducer array 904, generating an interrogation signal with this element, and detecting a reflected signal in response to the interrogation signal, wherein the reflected signal is reflected by a skull of the patient. The calibration process of FIG. 24 further includes computing an in-situ variable based on the reflected signal. In certain instances, the calibration process of FIG. 24 further includes comparing the in-situ variable computed by controller to an external data. The calibration process of FIG. 24 further includes adjusting an ensonification pattern or an array placement of the ultrasonic transducer array 904, or both, based on the in-situ variable. In instances where the in-situ variable is compared to the external data, the calibration process includes adjusting an ensonification pattern or an array placement of the ultrasonic transducer array 904, or both, based on the result of the comparison. In certain embodiments, the calibration process of FIG. 24 is repeated until all, or at least a predetermined subset, of the elements of ultrasonic transducer array 904 are calibrated to optimal frequencies.

One or more of the calibration processes depicted in FIGS. 22-23 can be executed by a control circuit. In another embodiment, one or more of the calibration processes depicted in FIGS. 22-23 are executed by a combinational logic circuit. In yet another embodiment, one or more of the calibration processes depicted in FIGS. 22-23 are executed by a sequential logic circuit. These examples are, however, not limiting. The calibration processes depicted in FIGS. 22-23 can be executed by circuitry that can include a variety of hardware and/or software components and may be located in or associated with various suitable systems described by the present disclosure.

Upon administration of the sonosensitizer, the controller 902 may utilize a combination of different ultrasound treatments at different time points according to one embodiment. For example, shortly after administering the sonosensitizer, it may be beneficial to apply an initial ultrasound treatment that helps enhance further uptake of the sonosensitizer. Once uptake of the sonosensitizer is considered within an optimal window based on time duration since the initial ultrasound treatment or other means, additional ultrasound treatment(s) can then subsequently be initiated. Applying different ultrasound treatments at different time points post administration of the sonosensitizer both encourages further uptake of the sonosensitizer, as well as enhances overall therapeutic effects. In another embodiment, instead of initiating ultrasound treatment based on time duration since the sonosensitizer and/or ultrasound treatment was administered, an apparatus could be used to directly monitor patient specific uptake of the sonosensitizer, and subsequently apply ultrasound treatment(s) when uptake is considered to be in an optimal range.

Research indicates that sonodynamic therapy depends on the creating of reactive oxygen species. These reactive oxygen species react with other molecules and damage organelles in the cancer cell. To enhance the oxidative damage within the cancer cells, the patient could be monitored for the amount of dissolved oxygen within cancer the cells, and/or alternatively monitored for peripheral capillary oxygen saturation levels according to one embodiment. The oxygen monitoring is then used as an additional patient specific input to guide application of ultrasound treatment(s) when parameters are considered to be in an optimal range.

Having described various embodiments of a sonodynamic therapy system 900, 920, 950, 1000, 1100 and components of the sonodynamic therapy system 900, 920, 950, 1000, 1100, the disclosure now turns to a description of the present disclosure that is directed to various embodiments of enhancing a sonodynamic therapeutic treatment that can be attenuated and enhanced to further produce complementary adjuvant effects which enhance the destruction of targeted cells and/or tissues according to various embodiments.

According to some non-limiting embodiments of the present disclosure, the aforementioned apparatuses, systems, and methods for enhancing a sonodynamic therapeutic treatment can be attenuated and enhanced to further produce complementary adjuvant effects which enhance the destruction of targeted cells and/or tissues. For example, the treatments disclosed herein can reduce the level of ultrasonic energy required to destroy a targeted cell and/or tissue and therefore, can limit the ensuing damage to healthy cells of surrounding organs. Thus, the apparatuses, systems, and methods disclosed herein provide numerous technical improvements, including the efficient use of resources (e.g. ultrasonic energy) and an advantageous ability to preserve the patient's overall health (e.g. eliminating destructive cells and preserving healthy cells). According to some non-limiting embodiments of the present disclosure, the therapies disclosed herein can produce such improvements, because they utilize complementary therapies (e.g. supplementary oxygenation, immunotherapy, anti-inflammatory therapy, microbubble enhanced cavitation, electromagnetic energy, magnetic energy, magnetic stimulation, one or more mono-pole or bi-pole electrodes, an array of electrodes, hyperthermia, hypothermia, alternative sonosensitizers and/or sonosensitizers with nano-particle additives) to enhance the efficacy of the sonodynamic therapy, itself. In one embodiment, electromagnetic energy (e.g., light) complements the sonodynamic therapeutic treatment. In one embodiment, magnetic energy (e.g., use of oscillating magnetic fields and/or magnetic stimulation at frequencies and durations proximate the treatment site) complements the sonodynamic therapeutic treatment. In some embodiments, magnetism may be used for treatment such as to stimulate and/or alter electrical activity in the brain and nervous system, such as through application of magnetic fields to the nervous system to induce electric currents within the nervous system via electromagnetic induction through one or more magnets (e.g., permanent, temporary), electromagnetic coils, rotating magnets, or other magnetic devices. In various embodiments, ultrasound therapy and/or a magnetic alignment device is used with magnetism (e.g., magnets, magnetic stimulation, magnetic field, static magnetic field, oscillating magnetic fields) with various degrees of magnetic flux density, magnetic induction, magnetic flux, magnetic potential, magnetomotive force, magnetic flied strength, magnetizing force, magnetization, magnetic polarization, magnetic moment, magnetic dipole moment, susceptibility, permeability, relative permeability, demagnetization factor and the respective units (e.g., in tesla, gauss, maxwell, gilbert, ampere, oestead, emu, at values including 0.1, 0.5, 1, 2, 3, 4, 5, 6, 7, 8, 9, 10, 50, 100, 200, 300, 400, 500, 1000 and ranging from 0.1-1, 0.1-10, 1-10, 1-5, 5-10, 10-100, 100-200, 100-500, 400-1000, and other ranges therein). However, it shall be appreciated that the apparatuses, systems, and methods disclosed herein can further enhance the efficacy of the complementary therapy modalities, as will be discussed.

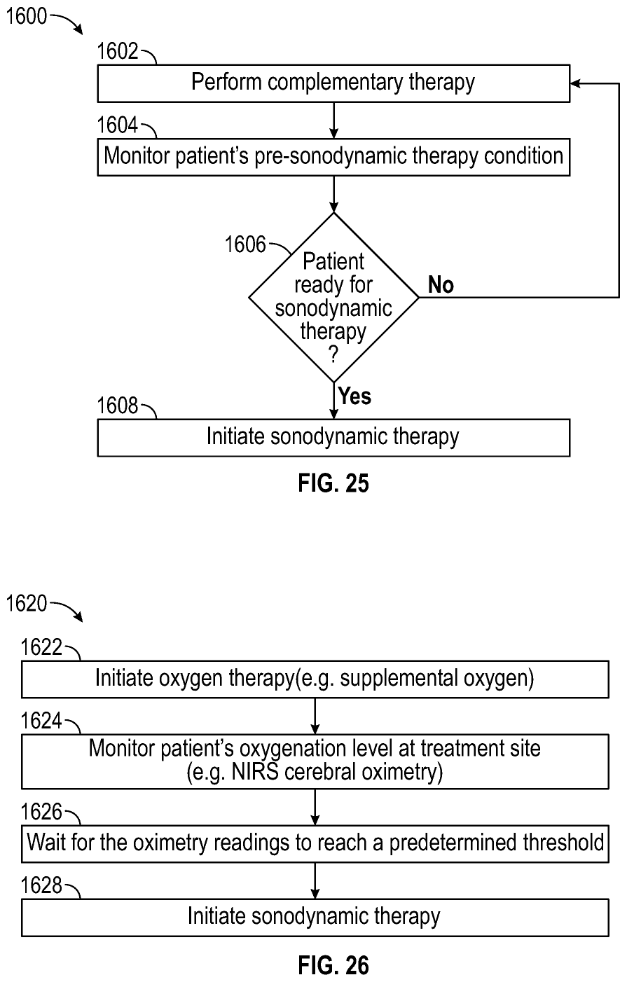

Referring now to FIG. 25, a flow diagram of a method 1600 of using a complementary adjuvant therapy to enhance the efficacy of a sonodynamic therapy is depicted in accordance with at least one non-limiting embodiment of the present disclosure. According to the non-limiting embodiment of FIG. 25, a clinician can perform a complementary therapy 1602 prior to initiating the sonodynamic therapy 1608. For example, after performing the complementary therapy 1602 on a patient, the clinician can monitor the patient's pre-sonodynamic therapeutic condition. In time, the clinician can assess the patient's pre-sonodynamic therapeutic condition 1606 to determine if the patient is properly prepared for the commencement of the sonodynamic therapy 1608. For example, the assessment 1606 can include a comparison of a biologic metric against a predetermined threshold, to asses if the complementary therapy has had the desired effect on the patient.

In further reference to FIG. 25, the threshold can be predetermined to correspond to the complementary therapy's ability to enhance the efficacy of the sonodynamic therapy according to one embodiment. Accordingly, the clinician can assess whether or not the patient requires additional therapy before the effects of the sonodynamic therapy can be optimized. If the patient is not exhibiting sufficient effects of the complementary therapy, the clinician can determine can decide to continue to perform the complementary therapy 1602 on the patient. For example, after monitoring the patient's progress 1604, the clinician may determine that a particular biologic metric does not sufficiently meet or exceed the predetermined threshold and the complementary therapy 1602 will continue. However, if the patient is exhibiting sufficient effects of the complementary therapy, the clinician can determine it is time to initiate the sonodynamic therapy on the patient 1608. For example, after monitoring the patient's progress 1604, the clinician may determine that a particular biologic metric sufficiently meets or exceeds the predetermined threshold, thereby concluding that the complementary therapy has had its effect and that ensuing sonodynamic therapy will result in enhanced efficacy.

Research indicates that sonodynamic therapies utilizes the creation and subsequent effects of ROS on organic molecules to target and destroy the organelles of an undesirable cell and/or tissue according to one embodiment. Accordingly, the method 1600 of FIG. 25 can include the use of a complementary therapy 1602 configured to enhance the oxidative damage within a targeted cell and/or tissue. The complementary therapy 1602 can include any number of means to increase the oxidative damage caused by ROS within the targeted cell and/or tissue. For example, the complementary treatment 1602 can be specifically configured to promote apoptosis, which accelerates the death of a targeted cell and/or tissue and inhibits reparative mechanisms within the targeted cell and/or tissue by enhancing oxidative damage.

Still referring to FIG. 25, the complementary therapy 1602 can be specifically configured to increase oxidative stress by inhibition and/or removal of anti-oxidants from the targeted cells and/or tissues according to one embodiment. For example, the oxidative stress can be enhanced by a complementary therapy's 1602 ability to reduce the amount of damage necessary to induce a cascade within a targeted cell and/or tissue, wherein the ensuing cascade accelerates cellular death. According to one non-limiting embodiment of the present disclosure, the oxidative stress can be enhanced by inhibiting cellular repair mechanisms that restore the cell after an oxidative stress is incurred. In some embodiments, a therapy that inhibits the ability of deoxyribonucleic acid (DNA) within the targeted cell and/or tissue to repair itself when the subsequent sonodynamic testing commences. The present disclosure contemplates an optimal use of complementary treatments that will maximize the degree of oxygenating sensitization incurred in targeted cells and/or tissues, while minimizing the targeted cell and/or tissue's sensitization to the sonodynamic therapy, itself.

According to another non-limiting embodiment of the present disclosure, the complementary therapy 1602 of FIG. 25 can be specifically tailored to enhance the amount of cavitation in one or more tissues. In one embodiment, sonodynamic therapies depend on the creation of reactive oxygen species, which can be produced by ultrasonically-induced cavitation. For example, the complementary therapy 1602 can include injecting the patient with microbubbles (e.g. micro-bubble ultrasound contrast agents). The injected microbubbles can be modified to accumulate on the targeted cells and/or tissues (e.g. tumors) upon injection, and further configured to cavitate upon exposure to ultrasound. According to yet another non-limiting embodiment of the present disclosure, the complementary therapy 1602 of FIG. 25 can further include injecting a patient with a drug that accumulates in a targeted cell and/or tissue such that the drug creates a nucleation site for enhanced cavitation. Nucleation sites can lower the pressure threshold required for cavitation. Accordingly, if the complementary therapy 1602 can be configured to create such nucleation sites, the conditions required or beneficial for cavitation—and subsequently, oxidative stress—can be preferentially developed within target tissues to occur at lower pressures in the targeted cells and/or tissue. This can enable the sonodynamic destruction of targeted cells and/or tissue 1608 to occur at a lower level of ultrasonic radiation, thereby preserving the unaffected surrounding cells, tissues, and/or organs.

According to other non-limiting embodiments of the present disclosure, the complementary therapy 1602 of FIG. 25 can further include ultrasonic imaging used to detect cavitation and/or the presence of bubbles occurring within various tissues. For example, an ultrasonic imaging transducer can apply pulses of increasing pressure to the anatomical subject to monitor for signals produced by and indicative of cavitation. As such, the ultrasonic transducer can focus ultrasonic pulses to various locations of the anatomical subject to assess the degree and effect of cavitation at multiple locations within a region of the anatomical subject. Acoustic radiation force impulse imaging (ARFI) is another feedback modality that can be utilized to characterize the target tissue and inform therapeutic ultrasound parameters. A clinician can utilize these pulses to assess the ultrasonic threshold for cavitation at the targeted cells and/or tissues, as well as throughout the rest of the anatomical subject. Accordingly, the sonodynamic therapy 1608 can be timed or modified based on the results of such cavitation monitoring, thereby enhancing its efficacy. In other non-limiting embodiments of the present disclosure, cavitation monitoring is performed with a cavitation monitoring device 830 (also referred to as a cavitation monitor 830). In various embodiments, a cavitation monitor is a probe, sensor, or device such as a hydrophone, microphone, transducer that measures activity in bubbles in a medium. In one embodiment, Passive cavitation detection (PCD) is a method to monitor microbubble activity during ultrasound exposure. In one embodiment, microbubble cavitation can be controlled by tuning ultrasound parameters (e.g., pressure amplitude, frequency, pulse repetition frequency, burst length, etc.). In one embodiment, low acoustic pressures induce stable cavitation (e.g., via alternation of expansion and shrinkage of microbubbles). In one embodiment, higher ultrasound intensities may induce inertial cavitation that leads to violent collapse and fragmentation of microbubbles accompanied by micro-jets and/or shock waves. In one embodiment, inertial cavitation quickly creates tissue damage, and thus real time cavitation monitoring can be implemented to alter or stop ultrasound treatment to avoid unwanted cavitational tissue damage. In some embodiments the cavitation monitor is configured to capture subharmonic, harmonic, and/or ultraharmonic frequencies of the fundamental ultrasound frequency (F) being delivered, for example in half harmonic increments 0.5 F, 1.5 F, 2F, 2.5 F, 3F, 3.5 F, 4F, 4.5 F, 5 F.

In one embodiment of a cavitation monitor, the time for microbubble distribution and the intensity of microbubble oscillation induced by ultrasound are directly related to their therapeutic efficacy and safety. In one embodiment, ultrasound waves set circulating microbubbles into nonlinear oscillations that generate specific harmonic components (e.g., subharmonic, harmonic, and/or ultra-harmonic). In one embodiment, at higher pressures, inertial cavitation collapses microbubbles, generating broadband emissions. The occurrence of subharmonic and ultra-harmonic frequencies is typically interpreted as a threshold for inertial cavitation that is associated with a risky oscillation regime of microbubbles. These unique acoustic signatures provide monitoring information about microbubble activity through passive cavitation detection. In various embodiments, feedback-control algorithms based on the detection of inertial cavitation, as well as other approaches based on the monitoring of specific frequency components such as harmonic, subharmonic and/or ultra-harmonic are indicators of efficient and or safe application of the ultrasound regime. One embodiment of a cavitation feedback control algorithm monitors the power spectral density (PSD) calculated using Welch's overlapped segment averaging spectral estimation. The PSD is monitored over time to produce a PSD Spectrogram. From the PSD spectrogram, line spectral content (e.g., a real time vertical cut through the spectrogram) can be monitored at the various subharmonic, harmonic, and/or ultra-harmonic increments. Furthermore, the broadband spectrum can be monitored from the PSD by masking the frequency bins corresponding to the fundamental frequency (F) and subharmonic, harmonic, and ultra-harmonic—leaving only broadband frequencies to evaluate for signs of inertial cavitation.

In one embodiment, a cavitation monitoring device 830 is configured to modulate the average acoustic pressure across a treatment target volumetric field, whereby upon detection of a degree of cavitation, the average acoustic pressure is increased to meet a minimum cavitation threshold. In one embodiment, a cavitation monitoring device 830 is configured to modulate the peak acoustic pressure across the volumetric field upon detection of a degree of cavitation, wherein the peak acoustic pressure is decreased to stay below a maximum cavitation threshold. In one embodiment, a passive cavitation monitoring device 830 is configured to modulate the average acoustic pressure across the volumetric field upon detection of a degree of cavitation. In one embodiment, a closed loop cavitation monitoring device 830 is a cavitation monitoring device configured to modulate the average acoustic pressure across the volumetric field upon detection of a degree of cavitation. Referring now to FIG. 26, a flow diagram of a method 1620 of using a supplemental oxygenating therapy to enhance the efficacy of a sonodynamic therapy is depicted in accordance with at least one non-limiting embodiment of the present disclosure. In one embodiment, the reactive oxygen species can be particularly configured to produce an intended reaction with predetermined molecules. Reactive oxygen species (ROS) as defined herein shall be given its ordinary meaning and shall also include free radicals, oxygen radicals, and/or unstable molecules that contain oxygen that react easily with other molecules. In some embodiments, ROS cause damage to proteins, DNA, RNA, and can cause cell death. For example, the reaction can include intentional damage (e.g. oxidative damage) to a targeted cell and organelles within the targeted cell. Accordingly, the method 1620 of performing a supplemental oxygenating therapy 1622 prior to initiating a sonodynamic therapy 1628 can increase the amount of dissolved oxygen within a targeted cell, thereby increasing the oxidative damage within the cancer cells and enhancing the efficacy of the sonodynamic therapy.

In further reference to the method 1620 of FIG. 26, a clinician can perform a supplemental oxygenating therapy 1602 prior to initiating the sonodynamic therapy 1608 according to one embodiment. The present disclosure contemplates various means of providing the patient with the supplemental oxygen 1622. For example, initiating the supplemental oxygenating therapy 1622 can include delivering a supplemental supply of oxygen into the respiratory system of a patient. According to other non-limiting embodiments of the present disclosure, initiating the supplemental oxygenating therapy 1622 can include introducing supplemental oxygen intravenously into the patient's bloodstream.

Still referring to FIG. 26, initiating the supplemental oxygenating therapy 1622 can include injecting the patient with gas-filled microparticles according to one embodiment. According to this non-limiting embodiment, the gas-filled microparticles include the supplemental oxygen, which are injected into the patient's bloodstream. The microparticles can be modified and specifically configured such that the supplemental oxygen, upon entering the patient's bloodstream, collect in at, or in the general area of, a targeted cell, tissue, and/or organ. Accordingly, the microparticles can be specifically configured to target the location of targeted tissue, thereby enabling a clinician to use lower radiation to destroy the targeted cell and/or tissue and thereby, minimizing the collateral damage of the sonodynamic procedure.

In further reference to FIG. 26, initiating the supplemental oxygenating therapy 1622 can include removing a portion of the patient's blood, oxygenating the removed portion of blood with the supplemental oxygen, and reinjecting the oxygenated sample of blood back into the patient (e.g. extracorporeal membrane oxygenation (EMCO)) according to one embodiment. According to other non-limiting embodiments of the present disclosure, initiating the supplemental oxygenating therapy 1622 includes administering (e.g., injecting) the supplemental oxygen directly into a targeted tissue, thereby enhancing the efficacy of the sonodynamic therapy in a manner similar to the use of the microparticles. According to still other non-limiting embodiments of the present disclosure, initiating the supplemental oxygenating therapy 1622 includes the delivery of oxygen at pressures above atmospheric pressure (hyperbaric oxygen therapy), thereby increasing increase in blood oxygen level in a targeted tissue function to promote healing and fight infection.

Still referring to FIG. 26, initiating the supplemental oxygenating therapy 1622 can include removing the use of a drug to enhance the oxygen concentration in a targeted cell and/or tissue according to one embodiment. For example, the present disclosure contemplates the use of an antihypoxic drug, such as trans sodium crocetinate, vinpocetine, 1-eburnamonine, vinconate, and/or vincamine, amongst others, which can be modified and/or packaged to specifically increase the levels of oxygen in a particular cell and/or tissue. According to still other non-limiting embodiments of the present disclosure, initiating the supplemental oxygenating therapy 1622 can include reducing the rate at which oxygen is used by the cell. For example, the clinician can reduce the patient's metabolism, thereby indirectly increasing the oxygenation of a targeted cell and/or tissue.

It shall be appreciated that the aforementioned embodiments describe specific means of initiating a supplemental oxygenating therapy 1622 for illustrative purposes only according to various embodiments. As such, it shall be further appreciated that the present disclosure contemplates the use of any supplemental oxygenating therapy 1622 that can assist in the oxygenation of a targeted cell and/or tissue. Accordingly, a clinician can use any such supplemental oxygenating therapy in the method 1620 of FIG. 26 to improve ROS destruction of targeted cells and/or tissue and therefore, enhance the efficacy of the subsequent sonodynamic therapy 1628.

In further reference to the method 1620 of FIG. 26, the clinician can monitor the patient's level of oxygenation 1624 according to one embodiment. According to the non-limiting embodiment of FIG. 26, blood oxygen levels can be monitored with an oximeter. For example, in the non-limiting embodiment where the targeted tissue and/or cells are located in the patient's brain, oxygenation of the brain could be monitored with cerebral oximetry. Accordingly, near-infrared spectroscopy (NIRS) cerebral oximetry can be used to monitor the brain oxygenation. According to other non-limiting embodiments of the present disclosure, various other light sources and/or detectors can be utilized in varying configurations to detect oxygen levels at different depths of the anatomical subject. According to still other non-limiting embodiments of the present disclosure, magnetic resonance imaging (MRI) and magnetic resonance guided focused ultrasound [MRgFUS] can be employed to asses and oxygen concentration within a targeted cell, tissue and/or organ, such as the brain. For example, blood oxygen level dependent (BOLD) contrast imaging can be used to visualize an anatomical structure (e.g. the brain) via functional magnetic resonance imaging (fMRI) to observe and/or treat with and magnetic resonance guided focused ultrasound [MRgFUS] targeted cells, tissues, and/or organs and detect oxygen levels therein.

However, it shall be further appreciated that the aforementioned embodiments describe specific means of monitoring a patient's oxygenation level at a treatment site 1624 for illustrative purposes only. As such, several embodiments provide the use of other means of monitoring a patient's oxygenation level at a treatment site 1624 to assess whether the patient is ready for a subsequent sonodynamic therapy 1628, thereby ensuring the sonodynamic therapy 1628 is enhanced according to one embodiment.

Still referring to FIG. 26, a clinician can continually monitor the oximetry readings and wait for them to meet or exceed a predetermined threshold 1626 prior to initiating sonodynamic therapy 1628 according to one embodiment. Similar to the embodiment depicted in FIG. 25, the clinician can assess the patient's pre-sonodynamic therapeutic condition 1606 to determine if the patient is properly prepared for the commencement of the sonodynamic therapy 1608. However, in the non-limiting embodiment of FIG. 26, the patient's pre-sonodynamic therapeutic condition corresponds to the oxygenation level at a treatment site. As such, the assessment 1626 can include a comparison of the oxygenation level at the treatment site against a predetermined oxygenation threshold, to assess if the complementary therapy has properly oxygenated the patient. Accordingly, the assessment of the patient's oxygen levels 1626 can provide the clinician with confidence in the efficacy of the subsequent sonodynamic therapy.

In further reference to FIG. 26, a clinician can decide to alter and/or time the initiation of sonodynamic therapy 1628 based, at least in part, on the assessment of the patient's level of oxygenation 1626 according to one embodiment. For example, if the clinician determines that the patient—or a target cell, tissue, and/or organ of the patient—is oxygenated below the predetermined threshold 1626, the clinician might decide to extend the oxygen therapy 1622 and delay the subsequent sonodynamic therapy 1628. According to some non-limiting embodiments, the determination 1626 can be specific to the targeted cell, tissue, and/or organ of the patient. For example, if the target tissue is a tumor located within the brain, an NIRS cerebral oximeter is placed on the head (perhaps a shaven head) as close to the tumor as possible, and the clinician makes the determination 1626 based on the specific oxygenation levels produced by the NIRS cerebral oximeter. Alternatively and/or additionally, the NIRS cerebral oximeter can be configured to continuously monitor the oxygenation of the targeted region and/or tissue until the predetermined threshold is met and/or exceeded. Accordingly, a system can be configured to autonomously notify the clinician and/or initiate the sonodynamic therapy 1628 when it is determined that the threshold has been met or is exceeded 1626.

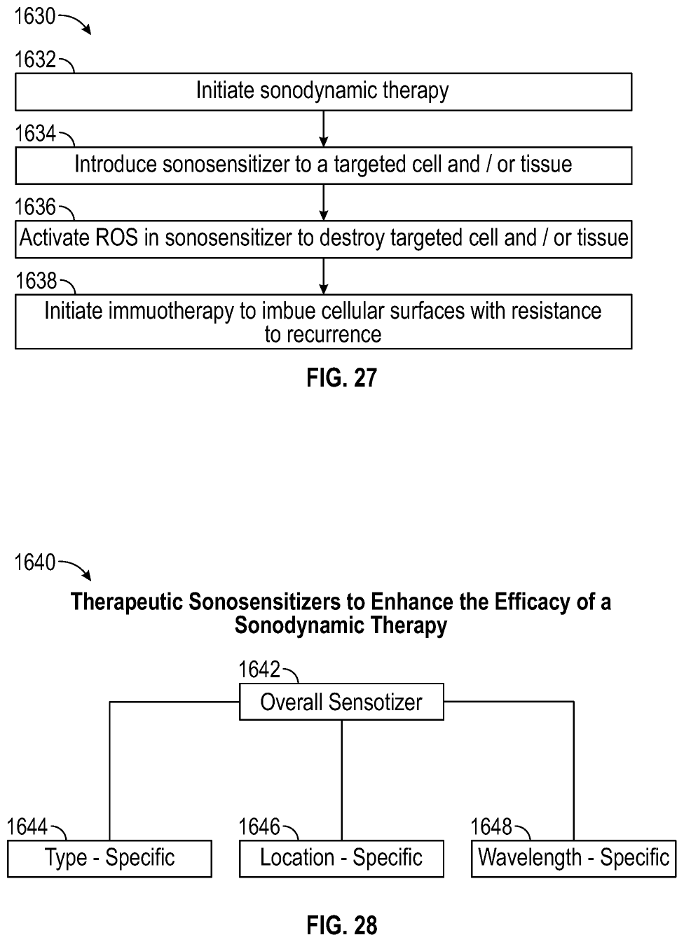

Referring now to FIG. 27, a flow diagram of a method 1630 of using immunotherapy to enhance the efficacy of a sonodynamic therapy is depicted in accordance with at least one non-limiting embodiment of the present disclosure. The method includes initiating a sonodynamic treatment 1632 to imbue an immunotherapeutic effect on targeted cells, tissues, and/or organs 1638. For example, the sonodynamic therapy can be used to damage the cancerous cells while mitigating damage to and enhancing the effectiveness of the cellular immunity.

For example, the method 1630 of FIG. 27 can employ a sonodynamic treatment 1632 that utilizes a specific sonosensitizer 1634 configured to inhibit the recurrence of a targeted cell and/or tissue that was destroyed via the sonodynamic therapy 1636 according to one embodiment. According to some non-limiting embodiments, the sonosensitizer utilized to destroy the targeted cells and/or tissue can indirectly produce an immunotherapeutic effect upon activation 1636, thereby resulting in a desired immunity (e.g. resistivity to the recurrence of the destroyed cell) in response to the sonodynamic therapy 1638. For example, damage-associated molecular patterns (DAMP) can result from the killing of targeted cells and/or tissues via sonodynamic therapy, leading to the creation of molecular patterns that elicit an immunotherapeutic response. Accordingly, the method 1630 of employing an enhanced sonodynamic therapy depicted in FIG. 27 can fortify surrounding cells, tissues, and/or organs by altering and training them to resist a recurrence of the targeted cells and/or tissues.

Referring now to FIG. 28, a block diagram 1640 depicting various therapeutic sonosensitizers 1642, 1644, 1646 configured to enhance the efficacy of a sonodynamic therapy is depicted in accordance with at least one non-limiting embodiment of the present disclosure. In one embodiment, sonodynamic therapeutic drugs—or sonosensitizers—can be specifically configured to improve accumulation on targeted cells and/or tissues and to produce an enhanced cytotoxic effect. For example, sonosensitizers can be specifically configured to improve the sonosensitizer's acoustic cavitation as well as the associated thermal, chemical or luminescent phenomena, all of which can improve enhance accumulation and acoustic reactivity during sonodynamic therapies.

According to the non-limiting embodiment of FIG. 28, an overall sonosensitizers configuration 1642 can include a type-specific sonosensitizer 1644, a location-specific sonosensitizer 1646, a wavelength-specific sonosensitizer 1648, and/or any combination thereof according to one embodiment. The sonosensitizers 1642, 1644 can be either experimental or approved by the FDA or other regulatory agency. Although the non-limiting embodiment of FIG. 28 depicts an overall sonosensitizer 1642 including a combination of sonosensitizers 1644, 1646, 1648, the present disclosure further contemplates embodiments in which the overall sonosensitizer 1642 includes a single specifically tailored sonosensitizer 1644, 1646, 1648 to achieve the desired effect. For example, the overall sonosensitizer 1642 can be specifically configured to target a specific type of cell, in a specific location of an anatomical subject, and react to ultrasonic stimulation from the transducers including a specific wavelength to improve the accumulation of overall sonosensitizers 1642 on a targeted cell and/or tissue and improve the acoustic reactivity of the overall sonosensitizer 1642.

For example, the overall sensitizer 1642 can be specifically configured to target a wound, ulcer, abscess, tumor, or any combination thereof according to one embodiment. The overall sonosensitizer 1642 can be further configured to target any of the aforementioned types of cells regardless of their relative position in the patient's body, and to react to a particular wavelength based on the location, therefore improving the destruction of targeted cells and/or tissues while leaving surrounding cells and/or tissues unharmed. Accordingly, an operating clinician can use the overall sonosensitizer 1642 to tailor the sonodynamic therapy based on the specific implementation and/or intended use. As such, the design of an overall sonosensitizer 1642, itself can enhance the efficacy of the sonodynamic therapy.

In further reference to FIG. 28, the overall sonosensitizer 1642 or any of the specifically tailored sonosensitizers 1644, 1646, 1648 can include a nanoparticle sonosensitizer according to one embodiment. Nanoparticle sonosensitizers can be used for their beneficial photocatalytic or sonocatalytic properties, which catalyze a reaction that produces reactive oxygen species. For example, titanium dioxide (TiO2), can be used to attenuate and/or regulate a desired cytotoxic effect. As such, the overall sonosensitizer 1642 can be specifically tailored to reduce toxicity, increase biodegradability, and improve cell and/or tissue targeting.

According to one non-limiting embodiment of the present disclosure, the sonodynamic therapy can be further enhanced to increase the concentration of protoporphyrin IX (PpIX) by limiting how much of it gets converted into Heme. For example, at least one of the overall sonosensitizer 1642, or any of the specifically tailored sonosensitizers 1644, 1646, 1648 of FIG. 28 can include 5-aminolevulinic acid (5-ALA). Amongst other things, 5-ALA can be utilized as a prodrug to induce the accumulation of PpIX in targeted cells and/or tissues. PpIX can induce cellular damage when exposed to ultrasonic wavelengths. 5-ALA is used in the endogenic production of a Heme group. For example, the final process of the Heme biosynthesis pathway includes inserting an iron ion into PpIX to form Heme, which is accomplished with ferrochelatase. Glioblastoma multiforme (GBM) has lower expression of the gene that produces ferrochelatase, which is why PpIX accumulates in GBM. ALA sonodynamic therapy (SDT) might be enhanced by further inhibiting the action of ferrochelatase. In some embodiments, iron ions are removed from some target cells to reduce the production of Heme and thus increase the concentration of PpIX. In other embodiments, the enzyme ferrochelatase removed from the cell. In other embodiments, a drug is delivered to reduce or eliminate the activity of ferrochelatase. In some embodiments, a sonosensitizer and/or product thereof accumulates in a tumor cell. In some embodiments, a sonosensitizer and/or product thereof accumulates in a mitochondria of a tumor cell. Gliomas, glial cells and/or astrocytomas are targeted and treated (e.g., selectively or preferentially) in several embodiments.

In various embodiments, the sonodynamic therapy techniques described in this disclosure may be adapted to other parts of the body. These other parts of the body may be accessed through a natural orifice (mouth, nasal cavity, ear, anus, vagina) or minimally invasive processes such as intravascular access. Implantable ultrasound devices that are at least partially implantable may also be used. The sonodynamic therapy device may be specifically adapted to have a flexible, navigable catheter shaft to reach tumors in specific organs such as liver, stomach, breast, or lungs, for example. The sonodynamic therapy device may be adapted to wrap around the torso or limb and may be employed to treat osteosarcoma into the bone.

In various embodiments, the sonodynamic therapy techniques described in this disclosure may be adapted for use with adjuvant therapies. The disclosed sonodynamic therapy techniques may be employed in other cancer therapies including chemotherapy, immunotherapy, radiotherapy, HIFU/hyperthermia. Further, the disclosed sonodynamic therapy techniques employ additional drugs which increase oxygen in the brain or increase oxygen in a brain tumor to a preferential oxygen concentration to provide an effective sonodynamic therapy. The disclosed sonodynamic therapy techniques may employ a sensitizer which is modified or encapsulated to effectively target a tumor. The disclosed sonodynamic therapy techniques may deliver O₂ systematically with nose tubes. The disclosed sonodynamic therapy techniques may employ multiple sensitizers in conjunction and may include the introduction of gas bubbles into the tumor to oxygenate the tumor, create more cavitation, and provide a possible contrast mechanism for imaging.

In various embodiments, the sonodynamic therapy techniques described in this disclosure may be adapted for use with ultrasound imaging according to one embodiment. The process may include the addition of a contrast agent for ultrasound which goes to the tumor. In various embodiments, CT, X-Ray, MRI, or other imaging may be used.

As used herein a processor or processing unit is an electronic circuit which performs operations on some external data source, usually memory or some other data stream. The term is used herein to refer to the central processor (central processing unit) in a system or computer systems (e.g., systems on a chip (SoCs)) that combine a number of specialized “processors.”

As used herein, a system on a chip or system on chip (SoC or SOC) is an integrated circuit (also known as an “IC” or “chip”) that integrates all components of a computer or other electronic systems. It may contain digital, analog, mixed-signal, and often radio-frequency functions-all on a single substrate. A SoC integrates a microcontroller (or microprocessor) with advanced peripherals like graphics processing unit (GPU), Wi-Fi module, or coprocessor. A SoC may or may not contain built-in memory.

As used herein, a microcontroller or controller is a system that integrates a microprocessor with peripheral circuits and memory. A microcontroller (or MCU for microcontroller unit) may be implemented as a small computer on a single integrated circuit. It may be similar to a SoC; an SoC may include a microcontroller as one of its components. A microcontroller may contain one or more core processing units (CPUs) along with memory and programmable input/output peripherals. Program memory in the form of Ferroelectric RAM, NOR flash or OTP ROM is also often included on chip, as well as a small amount of RAM. Microcontrollers may be employed for embedded applications, in contrast to the microprocessors used in personal computers or other general purpose applications consisting of various discrete chips.

As used herein, the term controller or microcontroller may be a stand-alone IC or chip device that interfaces with a peripheral device. This may be a link between two parts of a computer or a controller on an external device that manages the operation of (and connection with) that device.

Any of the processors or microcontrollers described herein, may be implemented by any single core or multicore processor such as those known under the trade name ARM Cortex by Texas Instruments. In one embodiment, the processor may be a LM4F230H5QR ARM Cortex-M4F Processor Core, available from Texas Instruments, for example, comprising on-chip memory of 256 KB single-cycle flash memory, or other non-volatile memory, up to 40 MHz, a prefetch buffer to improve performance above 40 MHz, a 32 KB single-cycle serial random access memory (SRAM), internal read-only memory (ROM) loaded with StellarisWare® software, 2 KB electrically erasable programmable read-only memory (EEPROM), one or more pulse width modulation (PWM) modules, one or more quadrature encoder inputs (QEI) analog, one or more 12-bit Analog-to-Digital Converters (ADC) with 12 analog input channels, details of which are available for the product datasheet.

In one embodiment, the processor may comprise a safety controller comprising two controller-based families such as TMS570 and RM4x known under the trade name Hercules ARM Cortex R4, also by Texas Instruments according to one embodiment. The safety controller may be configured specifically for IEC 61508 and ISO 26262 safety critical applications, among others, to provide advanced integrated safety features while delivering scalable performance, connectivity, and memory options.

As used herein, the terms “component,” “system,” “module” and the like can refer to a computer-related entity, either hardware, a combination of hardware and software, software, or software in execution, in addition to electro-mechanical devices. For example, a component may be, but is not limited to being, a process running on a processor, a processor, an object, an executable, a thread of execution, a program, and/or a computer. By way of illustration, both an application running on computer and the computer can be a component. One or more components may reside within a process and/or thread of execution and a component may be localized on one computer and/or distributed between two or more computers.

As used herein, the term control circuit may be any stand alone or combination electronic circuit such as, for example, a processing unit, processor, microcontroller, microcontroller unit, controller, digital signal processor (DSP), programmable gate array (PGA), field PGA (FPGA), programmable logic device (PLD), system on chip (SoC), application specific integrated circuit (ASIC), graphics processing unit (GPU), and the like. According to various embodiments, process flow diagrams described herein may be implemented by a digital device such as a control circuit.

Although the various embodiments of the present disclosure describe instruction handling and distribution in the context of execution units and logic circuits, other embodiments of the present disclosure can be accomplished by way of data and/or instructions stored on a machine-readable, tangible medium, which when performed by a machine cause the machine to perform functions consistent with at least one embodiment. In one embodiment, associated functions of the present disclosure are embodied in machine-executable instructions. The instructions can be used to cause a general-purpose or special-purpose processor that is programmed with the instructions to perform the steps of the functions described in the present disclosure. Embodiments of the present disclosure may be provided as a computer program product or software which may include a machine or non-transitory computer-readable medium having stored thereon instructions which may be used to program a computer (or other electronic devices) to perform one or more operations according to embodiments of the present disclosure. Alternatively, functions according to the present disclosure might be performed by specific hardware components that contain fixed-function logic for performing the functions, or by any combination of programmed computer components and fixed-function hardware components.

Instructions used to program logic to perform various disclosed embodiments can be stored within a memory in the system, such as DRAM, cache, flash memory, or other storage according to one embodiment. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, Compact Disc, Read-Only Memory (CD-ROMs), and magneto-optical disks, Read-Only Memory (ROMs), Random Access Memory (RAM), Erasable Programmable Read-Only Memory (EPROM), Electrically Erasable Programmable Read-Only Memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

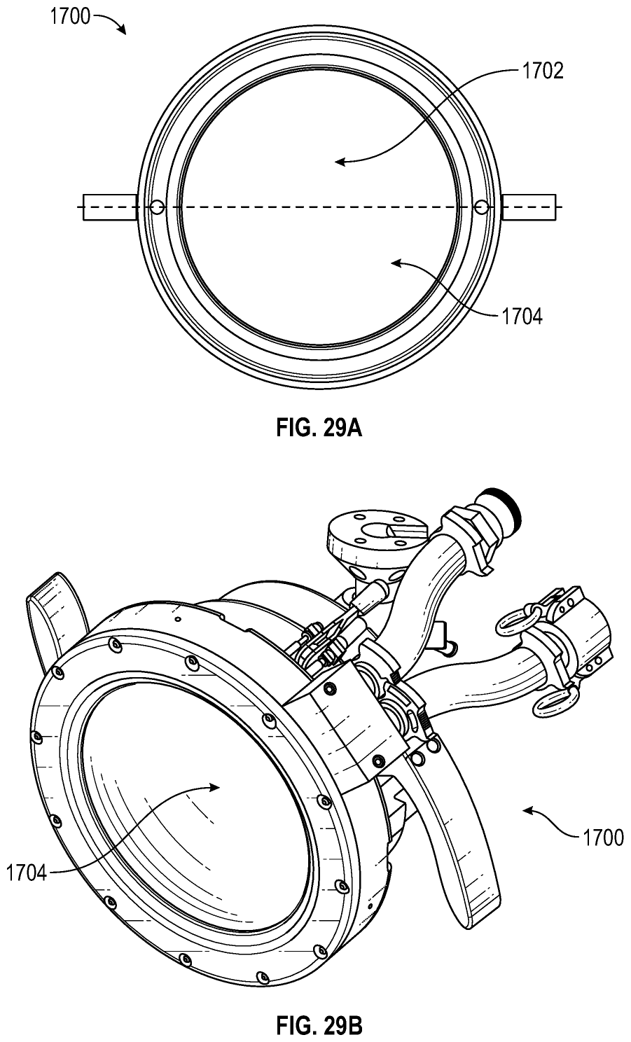

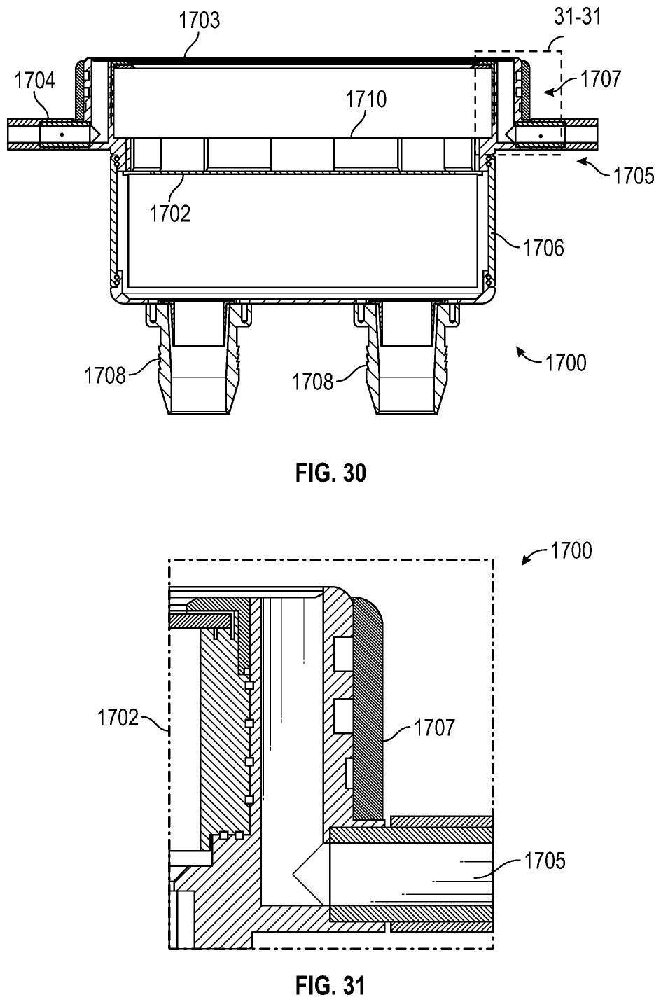

In various embodiments, an ultrasound transducer system 1700 comprises an array 1702 of transducer elements configured to emit an acoustic wave through an elastic, flexible membrane 1703 configured to conform to a shape of a portion of a body (e.g., head, skull, hips, abdomen, arm, leg, torso, back, waist, neck, etc.) for treatment. Referring now to FIGS. 29A, 29B, and 30-58 illustrate schematic views of an ultrasound transducer system 1700 according to at least one embodiment of the present disclosure. In one embodiment, the ultrasound transducer system is a transcranial sonodynamic therapy device 1700 configured for placement over the head of a patient.

In one embodiment, an acoustic wave 200, 312, 314, 1202, 1204, 1206, 1208, 1210, 1222, 1224, 1226, 1228 can be planar or defocused to minimize the spatial variation of the acoustic wave intensity in the brain. In various embodiments, the array 1702 comprises multiple transducer elements 150a-150h, 410, 420, 452, 454 that can be individually energized to produce a variety of acoustic waves, according to at least one embodiment of the present disclosure. In one embodiment, the array may be implemented as a single transducer comprising multiple piezoelectric elements with acoustically/electrically-independent sections arranged in the array 1702. The transducer elements 150a-150h can be arranged in an array 1702 to produce converging, diverging, or planar, acoustic waves. In one embodiment, one or more of the individual elements 150a-150h includes a flat, planar emitting surface that produces a planar acoustic wave. In one embodiment, the transducer element is flat, which may help reduce manufacturing costs.

In one embodiment, the element is made of a material with higher acoustic impedance than the target medium (water/tissue). Accordingly, an acoustic wave originates in the high-impedance material and transitions to the low-acoustic impedance target medium causing the acoustic wave to “defocus” or diverge to the target tissue.

In various embodiments, the transducer elements 150a-150h, 410, 420, 452, 454 can be activated in a predetermined sequence to selectively generate convergent/divergent/planar acoustic waves, such as, for example, a divergent acoustic wave. The transducer elements 150a-150h, 410, 420, 452, 454 may be energized in an order to produce a diverging acoustic wave using equal or different time delays. The transducer elements 150a-150h can be interchangeably configured to transmit or receive acoustic waves.

In one embodiment, the transducer elements 150a-150h, 410, 420, 452, 454 are arranged in a 2-dimensional (2D) grid array. In one embodiment, one or more elements in the 2-dimensional (2D) grid array includes at least one flat, planar emitting surface that produces a planar acoustic wave. Each transducer element of the 2D grid transducer array can be driven with a same signal or a different signal. In one embodiment of producing a defocused diverging acoustic wave from the dimensional (2D) grid array, the acoustic wave produced by the outer elements may be progressively more delayed relative to the inner element.

In one embodiment, the transducer element may be made of piezoelectric or single crystal material which converts electrical energy to ultrasonic energy. The transducer element also can receive back ultrasonic energy and convert it to electrical energy. Each transducer element may be selectively energized and actuated to produce convergent, divergent, or planar acoustic waves. In one embodiment, the transducer element has a zero vergence to produce a planar acoustic wavefront that does not converge or diverge. In one embodiment, a single converging/diverging acoustic wavefront may be produced by multiple elements of a transducer. In various embodiments, ultrasonic transducer element shapes include circular or disc shapes and concentric arrangements of elements. In various embodiments, ultrasonic transducer element shapes include square, rectangular, circular or disc shapes and various clustered grid arrangements of elements. In various embodiments, the arrangement of transducer elements may be any of the embodiments disclosed herein, including any of the embodiments and equivalents described in FIGS. 29-41.

In various embodiments, ultrasound transducer system 1700 includes a cooling system 1710. In one embodiment, the cooling system 1710 includes a fluid input tube 1704 and a fluid output tube 1705. In one embodiment, the fluid is water. In one embodiment, a membrane 1703 extends along an entry plane of the fluid input tube 1704 and along an exit plane of the fluid output tube 1705, forming a fluid port relief gap to cool the membrane 1703 with circulating fluid. The membrane 1703 is configured as any of the membrane embodiments herein. In one embodiment, the ultrasound transducer system includes a transducer housing 1706 attached to the array 1702 of transducer elements. In one embodiment, the transducer housing comprises one or more electrical connection ports 1708. In one embodiment, the cooling system 1710 comprises a bezel 1707 attached to the membrane 1703, fluid input tube 1704, and fluid output tube 1705. In one embodiment, the cooling system 1710 bezel 1707 is removably attachable to and detachable from the transducer housing 1706. In one embodiment, the membrane 1708 is detachable and replaceable. In one embodiment, the bezel 1707 is detachable and replaceable. In one embodiment, the cooling system 1710 is detachable and replaceable. In one embodiment, the cooling system 1710 bezel 1707 is permanently attached to the transducer housing 1706.

In one embodiment, the ultrasound transducer system 1700 comprises one or more handles for manually gripping and manually positioning the transducer system with respect to one or more treatment sites on a patient.

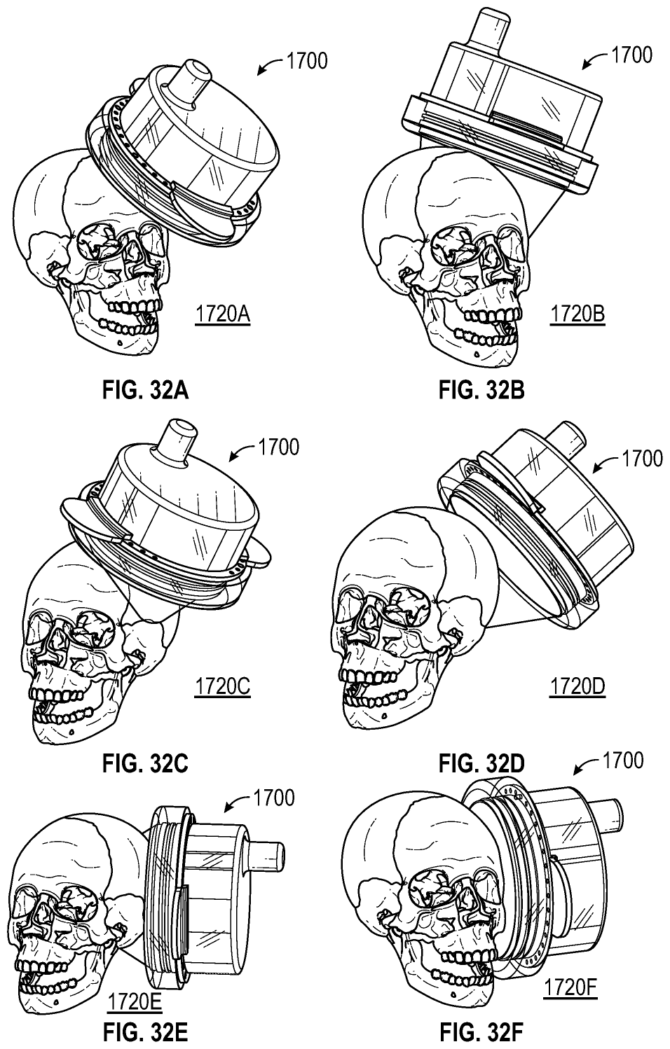

FIGS. 32A-32F are schematic images of placements of an ultrasound transducer system at multiple locations around a head for treatment of tissue in the head according to at least one embodiment of the present disclosure. At any one placement, one or multiple treatments may be performed. In one embodiment, the placements overlap. In one embodiment, the placements do not overlap. Moving the ultrasonic transducer array relative to the patient interface between a plurality of treatment positions or placements are configured to cause the ultrasonic transducer array to activate a sonosensitizer in a treatment region in the anatomical structure. In one embodiment, alignment of positions or placements is manual (e.g., by the system operator). In one embodiment, alignment of positions or placements is automated via robot arm or other mechanism (e.g., tracks, wheels, bearings, gears, rails, motors, actuators, hydraulics, pneumatics, magnetism, etc.) for linear, rotational, arcuate, curved, or other actuation.

Placement A 1720A illustrated at FIG. 32A comprises placement at the frontal bone of the skull for treatment embodiments including the frontal lobe.

Placement B 1720B illustrated at FIG. 32B comprises placement at a lateral embodiment of the frontal bone of the skull for treatment embodiments including the frontal and temporal lobes and thalamus.

Placement C 1720C illustrated at FIG. 32C comprises placement at the parietal bone of the skull for treatment embodiments including the parietal lobe.

Placement D 1720D illustrated at FIG. 32D comprises placement at an anterior embodiment of the parietal bone of the skull for treatment embodiments including the parietal and occipital lobes.

Placement E 1720E illustrated at FIG. 32E comprises placement at a lateral embodiment of the parietal bone of the skull for treatment embodiments including the occipital lobe, midbrain, and cerebellum.

Placement F 1720F illustrated at FIG. 32F comprises placement at the temporal bone of the skull for treatment embodiments including the temporal, parietal, and frontal lobes.

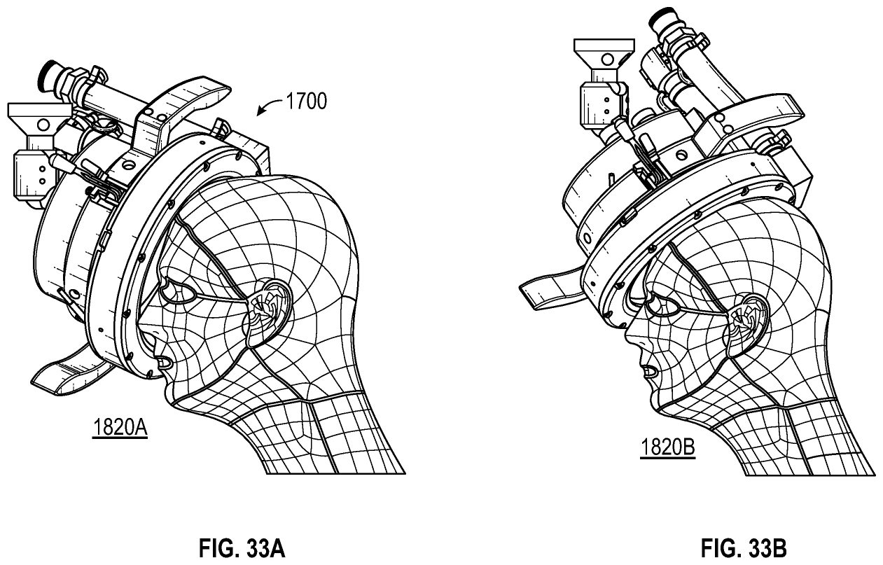

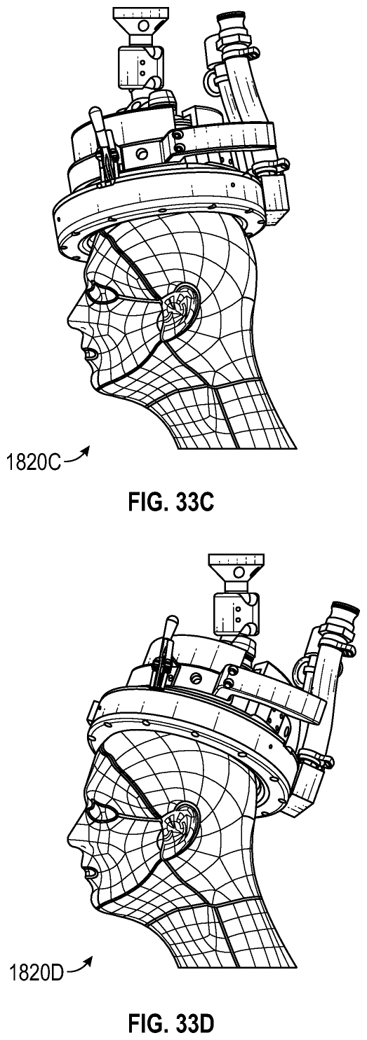

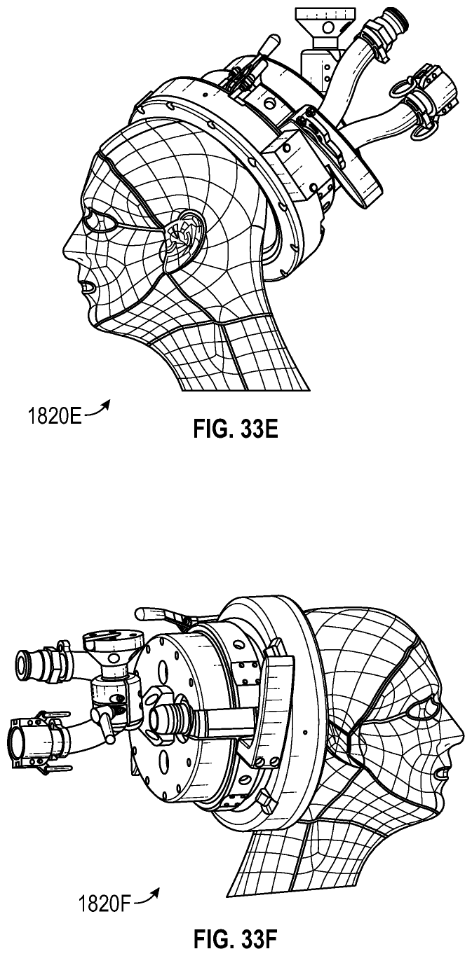

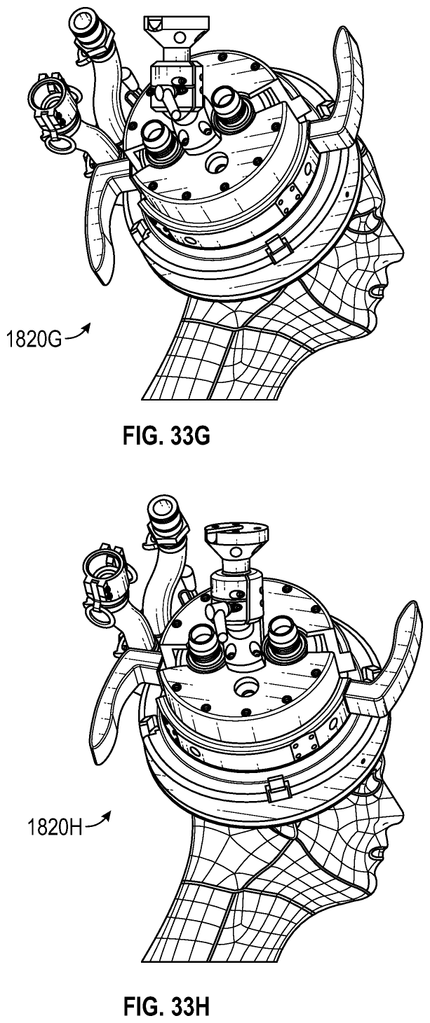

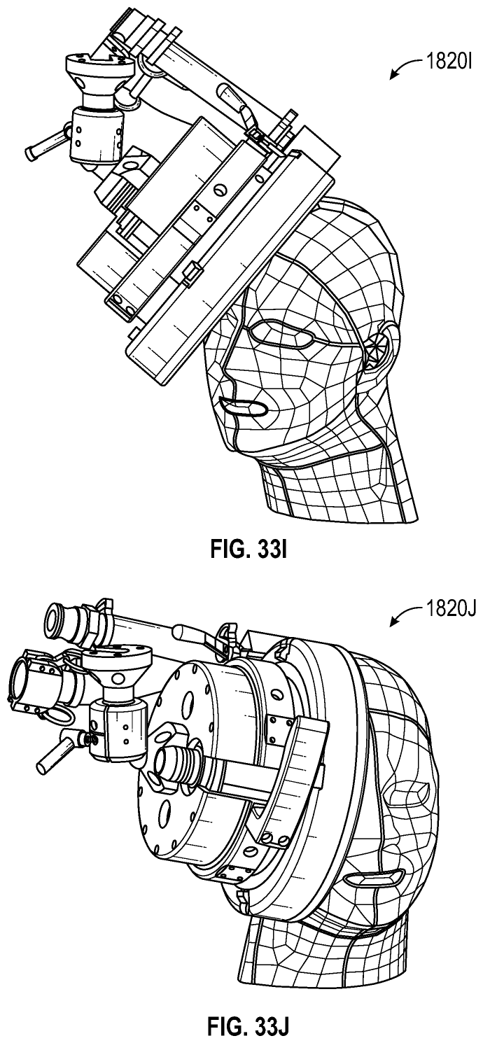

FIGS. 33A-33J are schematic images of placements of an ultrasound transducer system at multiple locations around a head for treatment of tissue in the head according to at least one embodiment of the present disclosure. At any one placement, one or multiple treatments may be performed. In one embodiment, the placements overlap. In one embodiment, the placements do not overlap. Moving the ultrasonic transducer array relative to the patient interface between a plurality of treatment positions or placements are configured to cause the ultrasonic transducer array to activate a sonosensitizer in a treatment region in the anatomical structure.

Placement A 1820A illustrated at FIG. 33A comprises placement at the frontal bone of the skull for treatment embodiments including the frontal lobe.

Placement B 1820B illustrated at FIG. 33B comprises placement at a lateral embodiment of the frontal bone of the skull for treatment embodiments including the frontal and temporal lobes and thalamus.

Placement C 1820C illustrated at FIG. 33C comprises placement at the parietal bone of the skull for treatment embodiments including the parietal lobe.

Placement D 1820D illustrated at FIG. 33D comprises placement at an anterior embodiment of the parietal bone of the skull for treatment embodiments including the parietal and occipital lobes.

Placement E 1820E illustrated at FIG. 33E comprises placement at an anterior embodiment of the parietal bone of the skull for treatment embodiments including the parietal and occipital lobes.

Placement F 1820F illustrated at FIG. 33F comprises placement at the temporal bone of the skull for treatment embodiments including the temporal, parietal, and frontal lobes.

Placement G 1820G illustrated at FIG. 33G comprises placement at a lateral embodiment of the parietal bone of the skull for treatment embodiments including the occipital lobe, midbrain, and cerebellum.

Placement H 1820B illustrated at FIG. 33H comprises placement at a lateral embodiment of the frontal bone of the skull for treatment embodiments including the frontal and temporal lobes and thalamus.

Placement 118201 illustrated at FIG. 33I comprises placement at the frontal bone of the skull for treatment embodiments including the frontal lobe.

Placement J 1820J illustrated at FIG. 33J comprises placement at the temporal bone of the skull for treatment embodiments including the temporal, parietal, and frontal lobes.

In various embodiments, an alignment device 850, 1900 is used in conjunction with the ultrasound transducer system 100 to help align the treatment with a treatment site of the patient. In various embodiments, alignment device 850 is an alignment device 1900. The alignment device 1900 comprises multiple marks or markings 1910 (e.g., fiducial markers, magnetic markers, etc.) to facilitate treatment, such as by demarking a grid, positions based on one or more anatomical features, and/or marking of the skin with indicators. In various embodiments, the alignment device 1900 is a pen, a marker, a ruler, a targeting template, a laser alignment device, a magnetic alignment device, an imaging system (e.g., camera, CT, MRI), and/or a motorized alignment system comprises a track and a gimbal for controlled and lor automated mechanical alignment of the ultrasound transducer system with a treatment site.

In one embodiment, an alignment device 1900 is drawn on a portion of the body of the patient using a pen, marker, or other writing device.

In one embodiment, an alignment device 1900 is a surgical ruler with marks/markers to assist in the measuring and marking of treatment sites on the patient. In one embodiment, treatment site locations are pre-operatively planned to accomplish full therapeutic coverage of the diseased organ.

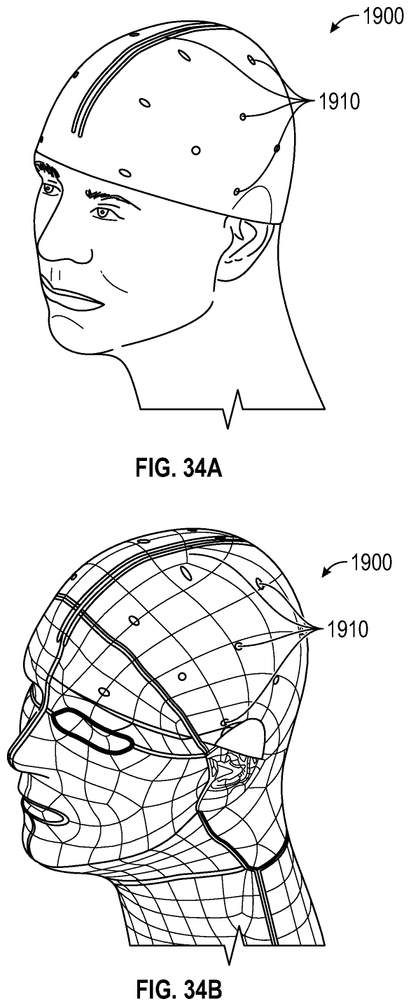

In one embodiment, alignment device 1900 is a targeting template placed on the patient to facilitate alignment of the transducer to the various treatment sites. In various embodiments, the targeting template 1900 is a wearable elastic template with one, two, three, four, five, six, ten, twelve, fifteen or more markers 1910 (e.g., fiducial markers, magnetic markers, etc.) to facilitate treatment, such as by demarking a grid, positions based on one or more anatomical features, and/or marking of the skin with indicators. In one embodiment, the targeting template includes measurement gradients that allow the user to customize treatment locations to patient specific anatomical size. In one embodiment, the targeting template 1900 is customized (e.g., custom 3D printed or formed, sewn, etc.) for customized fit to a particular patient. In some embodiments, the targeting template remains in place during ultrasound treatment. In some embodiments, the targeting template is made to be removable prior to ultrasound treatment. In various embodiments, a targeting template is elastic and configured for removable attachment to a portion of the body of the patient. In one embodiment, modular patient interfaces, arrays, transducers and/or transducer elements are configured for removable and customized placement with respect to the system and/or patient. In one embodiment, alignment device 1900 has marks corresponding to placement of modular patient interfaces, arrays, transducers and/or transducer elements are configured for removable and customized placement with respect to the system and/or patient. In various embodiments, a targeting template is an elastic cap 1900 configured to fit over the head of a patient, as shown in FIGS. 34A-34B. In one embodiment, the targeting template 1900 is a band configured to wrap around a head, neck, chest, torso, back, waist, leg, buttock, genital area or other body part.

In one embodiment, an alignment device 1900 is a motorized alignment system with a track and a gimbal to provide motion within or around a housing for alignment of the system to one or more treatment sites on the patient's body. In one embodiment, the motorized alignment system operates within a helmet (e.g., cap, headset, hat, skull cap, etc.) to automatically align to a plurality of positions and locations for treatment without needing manual manipulation of the system by an operator during a treatment. In one embodiment, alignment device 1900 is a motorized robot arm to provide alignment and placement of the treatment system in a position for one or more treatment sites on the patient's body.

In one embodiment, an alignment device 1900 comprises a laser attached to a housing of the at least one ultrasound array, wherein the laser is attached to a targeting system configured to locate and verify a position of an alignment feature of an anatomical landmark on the patient for alignment of the treatment of tissue of the patient.

In one embodiment, an alignment device 1900 comprises a magnetic alignment device. In one embodiment, the magnetic alignment device works with magnetic markers. In various embodiments, ultrasound therapy and/or a magnetic alignment device is used with magnetism (e.g., magnets, magnetic stimulation, magnetic field, static magnetic field, oscillating magnetic fields) with various degrees of magnetic flux density, magnetic induction, magnetic flux, magnetic potential, magnetomotive force, magnetic flied strength, magnetizing force, magnetization, magnetic polarization, magnetic moment, magnetic dipole moment, susceptibility, permeability, relative permeability, demagnetization factor and the respective units (e.g., in tesla, gauss, maxwell, gilbert, ampere, oestead, emu, at values including 0.1, 0.5, 1, 2, 3, 4, 5, 6, 7, 8, 9, 10 and ranging from 0.1-1, 0.1-10, 1-10, 1-5, 5-10, and other ranges therein).

In various embodiments, an authorization system 860 is configured to identify identification code(s) on a drug, pro drug, sonosensitizer, microbubble additive, and/or one or more components of the ultrasound transducer system 100 that is configured to operate with one or more components of (or the entire) the ultrasound transducer system 100.

In one embodiment, a code on a drug, pro drug, sonosensitizer, and/or microbubble additive is identified in order to authorize subsequent treatment using the ultrasound transducer system 100. In various embodiments, the drug, pro drug, sonosensitizer, and/or microbubble additive, and/or packaging container thereof has a unique identifier (e.g., RFID, identification code, security key, bar code, QR code, hologram, etc.). In one embodiment, the drug, pro drug, sonosensitizer, and/or microbubble additive is administered to a patient. In one embodiment, the ultrasound transducer system 100 identifies and matches the unique identifier code to an authorization code before authorizing a medical treatment using the ultrasound transducer system 100. In one embodiment, the ultrasound transducer system 100 identifies the identifier directly (via scan or machine reading of the identifier)—if the identification code matches, therapy is enabled; if the identification code does not match, therapy is disabled. In one embodiment, the ultrasound transducer system 100 identifies a patient's identification (e.g., patient code, wrist band, etc.), wherein the patient's identification is linked to the administration of the identifier from the coded drug, pro drug, sonosensitizer, and/or microbubble additive.

In one embodiment, a code on one or more components of the ultrasound transducer system 100 is identified in order to authorize subsequent treatment using the ultrasound transducer system 100. In various embodiments, the one or more components of the ultrasound transducer system 100 and/or packaging container thereof has a unique identifier (e.g., RFID, identification code, security key, barcode, QR code, hologram, etc.). In one embodiment, the one or more components of the ultrasound transducer system 100 is scanned or machine read by the ultrasound transducer system 100. In one embodiment, the one or more components of the ultrasound transducer system 100 is attached to the ultrasound transducer system 100. In one embodiment, the ultrasound transducer system 100 identifies and matches the unique identifier before authorizing a medical treatment using the ultrasound transducer system 100. In one embodiment, the ultrasound transducer system 100 identifies the identifier directly (via scan or machine reading of the identifier)—if the identification code matches, therapy is enabled; if the identification code does not match, therapy is disabled.

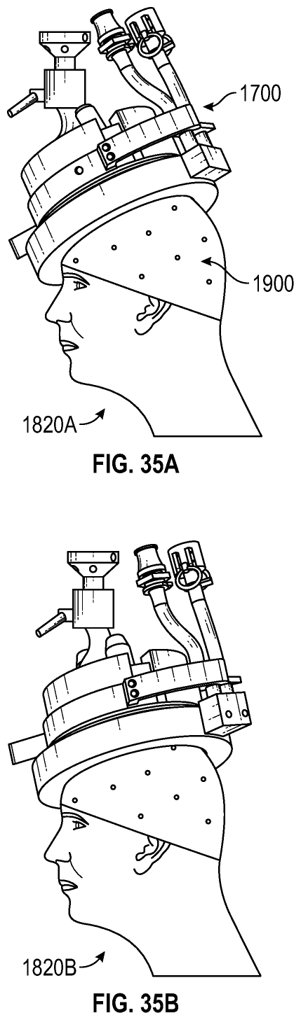

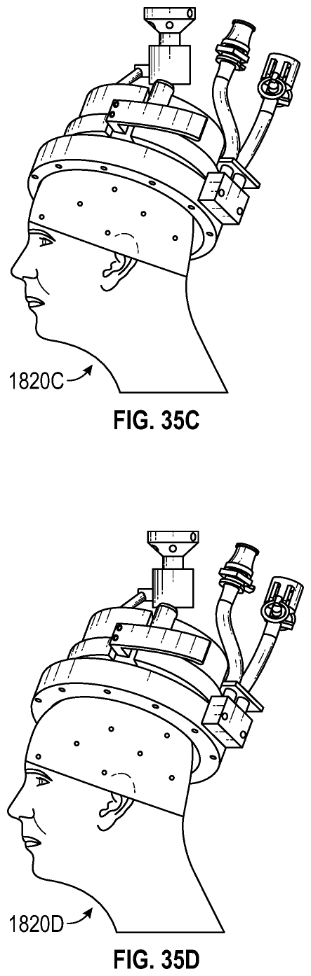

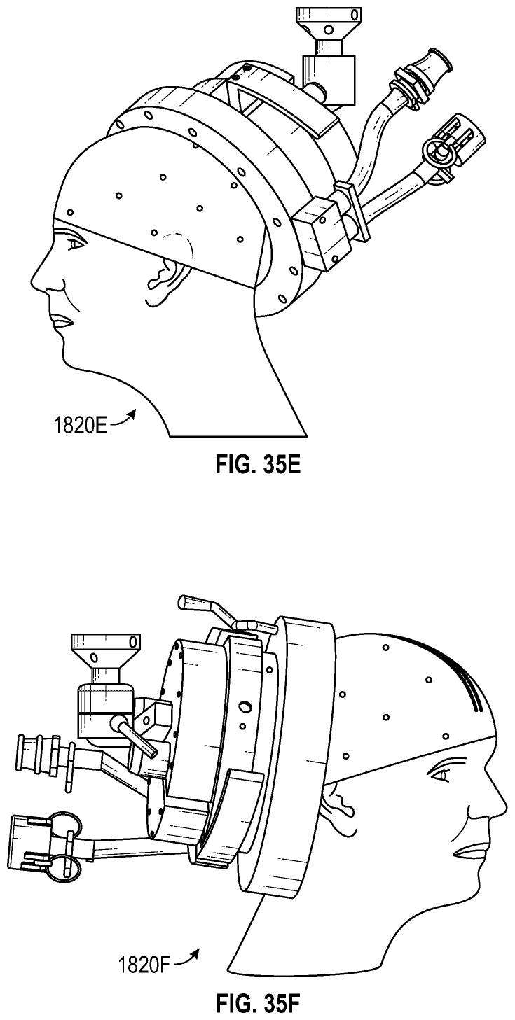

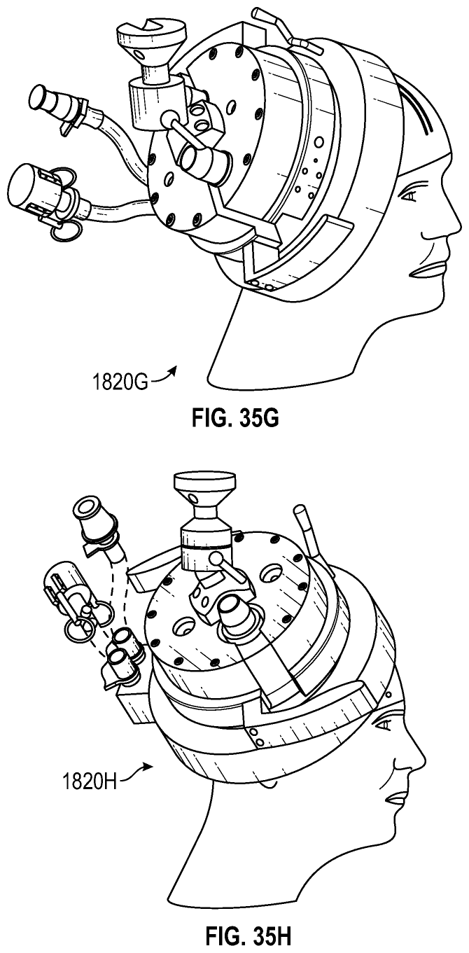

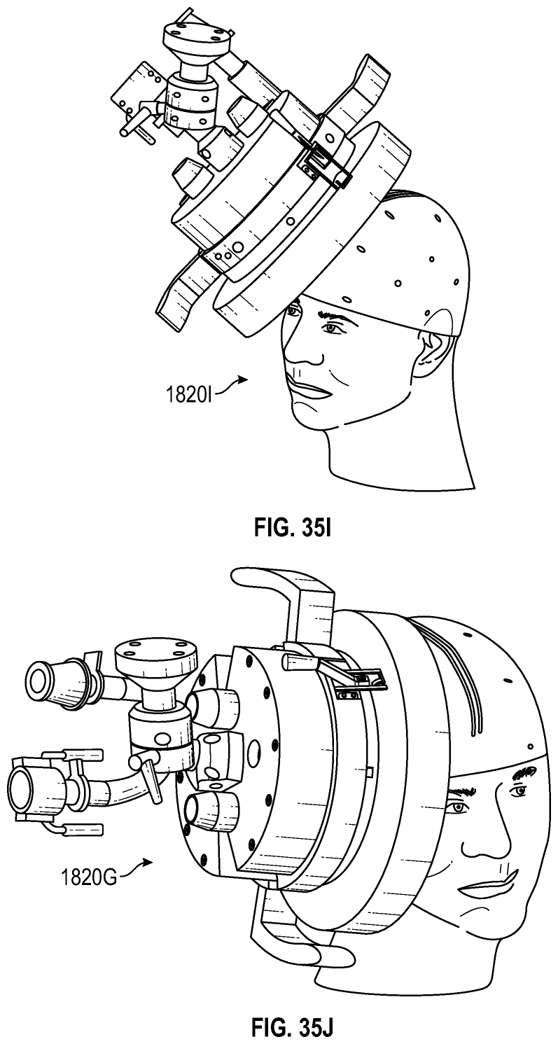

FIGS. 35A-35J are schematic images of placements of an ultrasound transducer system at multiple locations around a targeting template 1900 with a plurality of markers 1910 for treatment of tissue in the head according to at least one embodiment of the present disclosure. At any one placement, one or multiple treatments may be performed. In one embodiment, the placements overlap. In one embodiment, the placements do not overlap. Moving the ultrasonic transducer array relative to the patient interface between a plurality of treatment positions or placements are configured to cause the ultrasonic transducer array to activate a sonosensitizer in a treatment region in the anatomical structure.

Placement A 1820A illustrated at FIG. 35A comprises placement at the frontal bone of the skull for treatment embodiments including the frontal lobe.

Placement B 1820B illustrated at FIG. 35B comprises placement at a lateral embodiment of the frontal bone of the skull for treatment embodiments including the frontal and temporal lobes and thalamus.

Placement C 1820C illustrated at FIG. 35C comprises placement at the parietal bone of the skull for treatment embodiments including the parietal lobe.

Placement D 1820D illustrated at FIG. 35D comprises placement at an anterior embodiment of the parietal bone of the skull for treatment embodiments including the parietal and occipital lobes.

Placement E 1820E illustrated at FIG. 35E comprises placement at an anterior embodiment of the parietal bone of the skull for treatment embodiments including the parietal and occipital lobes.

Placement F 1820F illustrated at FIG. 35F comprises placement at the temporal bone of the skull for treatment embodiments including the temporal, parietal, and frontal lobes.

Placement G 1820G illustrated at FIG. 35G comprises placement at a lateral embodiment of the parietal bone of the skull for treatment embodiments including the occipital lobe, midbrain, and cerebellum.

Placement H 1820B illustrated at FIG. 35H comprises placement at a lateral embodiment of the frontal bone of the skull for treatment embodiments including the frontal and temporal lobes and thalamus.

Placement I 1820I illustrated at FIG. 35I comprises placement at the frontal bone of the skull for treatment embodiments including the frontal lobe.

Placement J 1820J illustrated at FIG. 35J comprises placement at the temporal bone of the skull for treatment embodiments including the temporal, parietal, and frontal lobes.

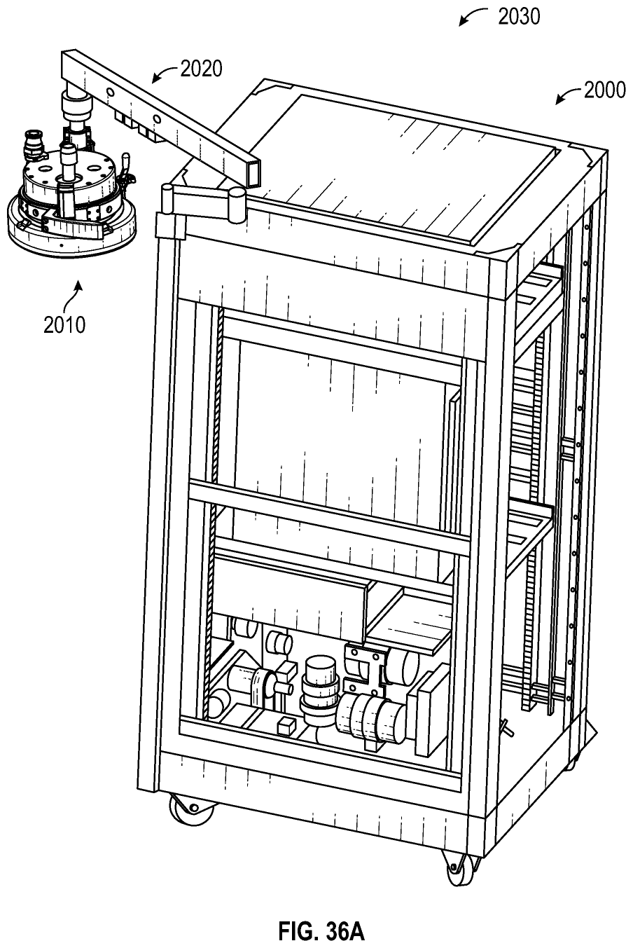

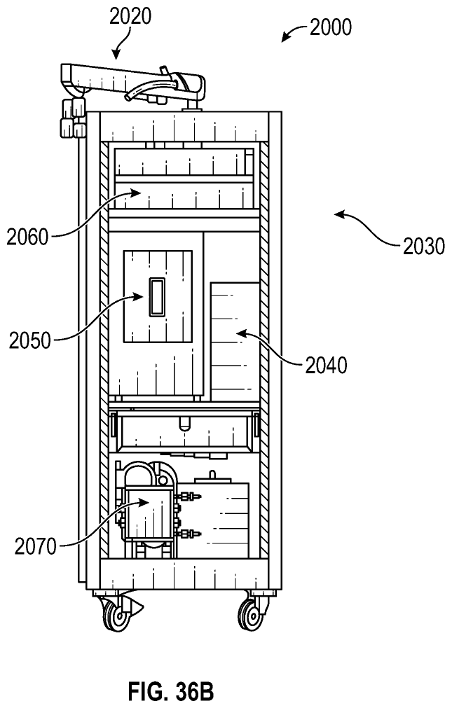

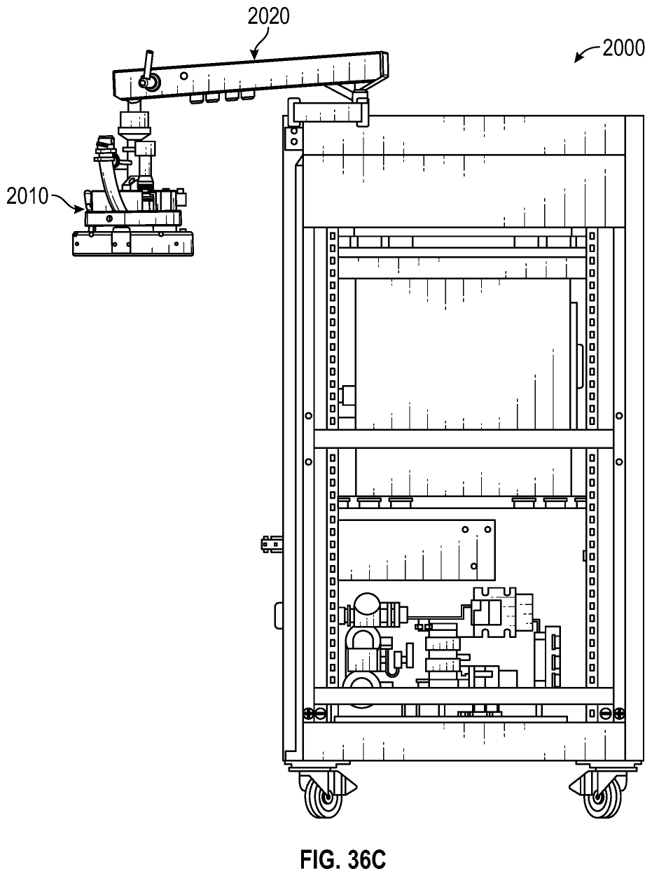

In various embodiments, a sonodynamic treatment system 2000 includes a transducer array 2010, support arm 2020, cart 2030, console/controller 2040, ultrasound generator 2050, user interface 2060, and/or a cooling fluid circulation unit 2070 according to any of the embodiments herein. In one embodiment, the support arm 2020 is counterbalanced to facilitate placement of the transducer array 2010 in one or more positions (e.g., 1, 2, 3, 4, 5, 6, 8, 10, 12, 15, 20 or more positions) during a sonodynamic treatment. FIGS. 36A-C illustrate views of one embodiment of a mobile sonodynamic treatment system 2000 according any of the embodiments of any of the components described herein.

### Example 1. Ultrasound Transducer System Producing a Normalized Acoustic Pressure Profile

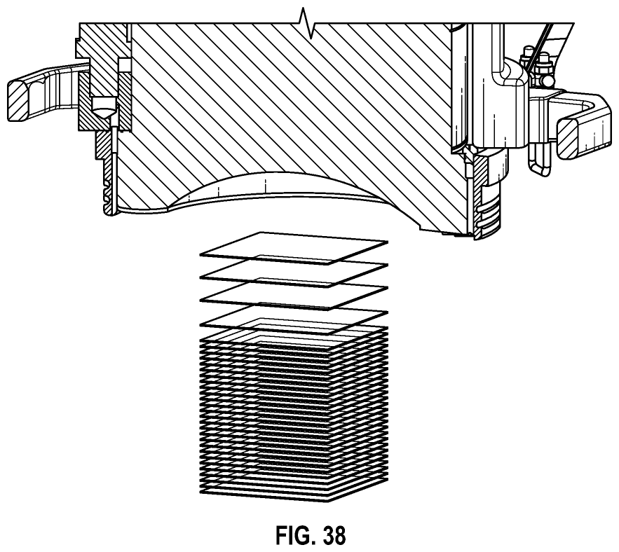

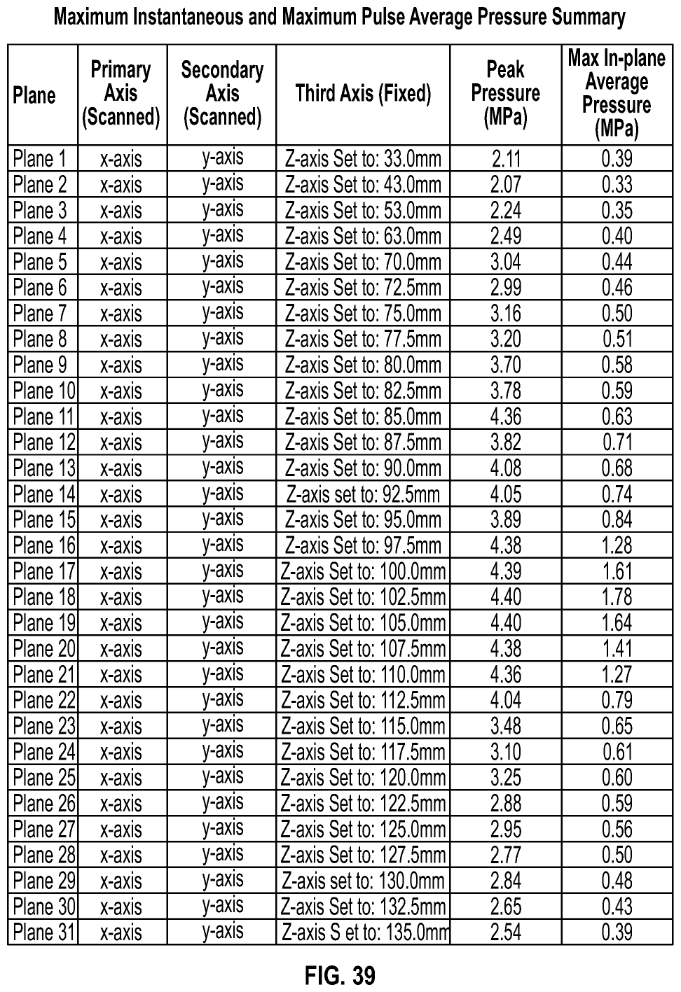

The following example illustrates one embodiment that should not be used to limit other variations and embodiments therein. An ultrasound transducer system 100 was activated and acoustic pressure was measured within via a volumetric scanning method described below. FIG. 38 represents a visual representation of volume scanning planes for measurement of pressure emitted at incremental distances from the ultrasound transducer system 100. To verify the peak pressure present in the field a volume scan consisting of multiple planes as listed in Table 1 (FIG. 39) from the apex of the transducer was performed. The resultant pressure values were based on maximum power settings, which were only applied at the thickest skull locations. The step size was 1.5 mm sampling within plane. In other embodiments, the step size could be 0.5, 1.0, 1.25, 1.75, 2.0, 2.5 mm or other values and ranges therein.

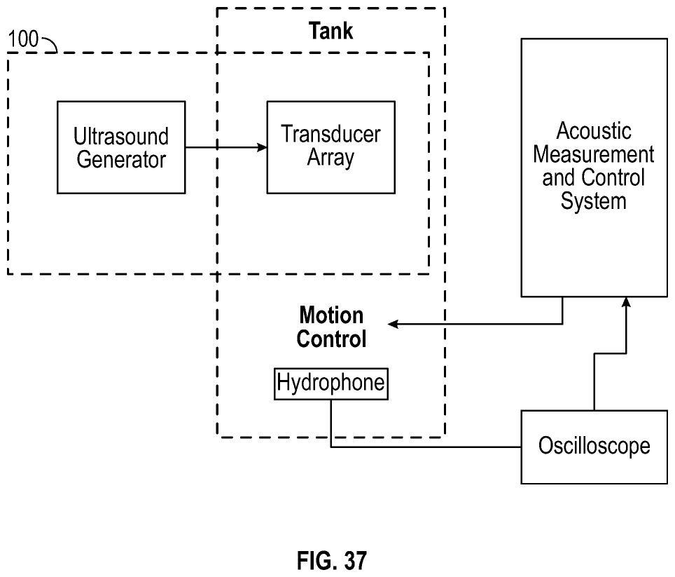

The experiment procedure started under initial conditions (prior to the start of the testing) at a known state, with a system set-up shown at FIG. 37. The transducer array was be positioned in the acoustical measurement tank with the firing face submerged in water, and all air bubbles were removed. Prior to all tests, the hydrophone was positioned in the tank at the focal point of the transducer array. This was defined as location x, y, z=0, 0, 83 mm. In other embodiments, locations can be used at any values for x, y, and/or z in a range of 1-100 mm.

The steps of the performed experiment included:
(1) the hydrophone was aligned to the acoustic axis by jogging the hydrophone positioner to the center of the transducer array with the tip of the hydrophone nominally at a depth of ˜30 mm from the transducer elements in the z-axis.
(2) the ultrasound transducer system 100 was set with the water circulating to output at 5% duty cycle.
(3) performed scan of plane 1 scan as defined in Table 1 (FIG. 39) to capture the field and save the results.
(4) repeated step (3) for all 31 planes in the volumetric characterization.

The test sample was performed on an embodiment of the ultrasound transducer system 100, with the Absolute Peak Pressure representing a measurement of the maximum absolute instantaneous pressure, and the Pulse Average Pressure representing the average absolute instantaneous pressure between the time when the time integral of the pulse intensity integral reaches 10% and 90% of its final value for each burst, then was averaged across all bursts.

The measurement instrumentation included an oscilloscope, a water tank (with degassed water), a stepper motor driven X-Y-Z micromanipulation system and a PC compatible computer for motion control and data collection from a software application. A separate software application was designed to calculate the appropriate acoustic parameters (pressure, intensity, pulse integral, etc.) for the acquired data. The system has undergone both installation qualification and operation qualification as well as software verification and validation. A capsule hydrophone with an in line preamplifier was used during testing. This hydrophone had an active diameter of 0.2 mm in combination with a non-submersible pre-amplifier designed to drive a 500 load. The hydrophone was calibrated with a frequency response that was smooth and flat with measurement uncertainty of 1 dB.

The complete measurement system comprised of the hydrophone with preamplifier, the oscilloscope, water tank and a stepper motor as shown in the embodiments shown in FIG. 37. The positioning system step resolution was 50 μm in all three directions. The positioning systems allowed the hydrophone to be scanned through the field at pre-defined coordinates for single point, line, plane or volume scans. The oscilloscope and stepper motor controller were interfaced with the PC via USB for automated positioning and capture and storage of the acoustical signals from the hydrophone.

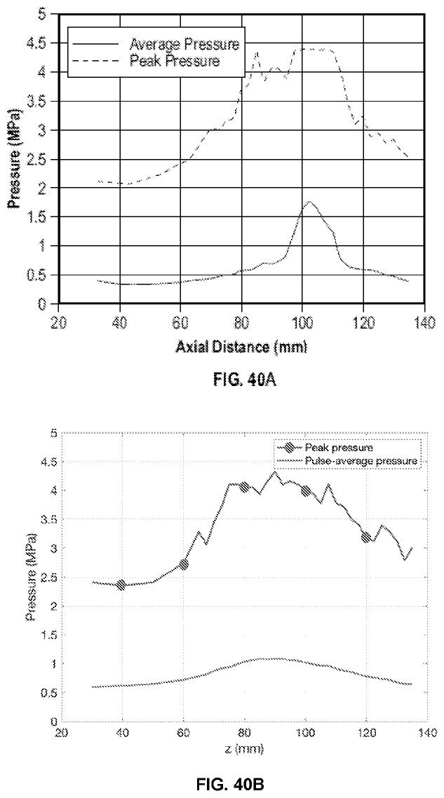

According to several embodiments, experimental data and results are presented at FIGS. 39, 40A and 40B. The planes were numbered according to the protocol. For the purposes of convention of plotting the planes the x-axis is the lateral dimension, the y-axis reflect the elevation direction and the z-axis the axial direction as it related to the transducer array. The array was excited with the therapeutic driving patterns and a long scope acquisition was acquired to capture the set of driving patterns. Hydrophone scanning was completed and the results analyzed for plotting of pressure and intensity fields as well as calculation of spatial peak parameters. FIG. 40A is a graphical representation of average and peak pressures by plane. The pulse average pressure was calculated as the average pressure in all pulses received. An embodiment of the ultrasound transducer system 100 driving patterns had the frequency swept from 700 kHz, 750 kHz, 900 kHz, 950 kHz, 1 MHz and 1.05 MHz and utilized a collection of 256 unique phase randomizations for each element in the array. In other embodiments, the ultrasound transducer system 100 driving patterns could have the frequency swept from 600 kHz, 650 kHz, 750 kHz, 800 kHz, 900 kHz, 950 KHz, 1 MHz, 1.05 MHz, 1.1 MHz, and/or 1.2 MHz and can use a collection of 2, 4, 8, 16, 32, 64, 128, 256, 512, 1024, and/or 2048 unique phase randomizations for each element in the array. Randomizing the phase across each element in the array was done to limit the possibility of standing waves and provide a diffuse acoustic field that continually redistributes the energy within the field. Thus, the peak instantaneous pressure at a location occurred for a fraction of a pulse width, and was therefore at an extremely low duty cycle compared to the overall duty cycle of the therapy. This driving regime also explains why the pulse average power is significantly lower than peak instantaneous pressure due to the unique phase randomized drive patterns.

The volumetric peak instantaneous pressure of 4.40 MPa occurred near the center of the 102.5 mm plane. The maximum pulse average pressure for a single plane was 1.78 MPa occurring at the 102.5 mm plane. The pulse averages across the volumetric field (average of all planes) was 0.73 MPa. In other embodiments, volumetric peak instantaneous pressure could be in a range of 1-8 MPa (e.g., 1, 1.5, 2, 2.5, 3, 3.5, 4, 4.5, 5, 5.5, 6, 6.5, 7, 7.5, and/or 8 MPa and other values therein). In other embodiments, maximum pulse average pressure for a single plane could be 0.1-5 MPa (e.g., 0.1, 0.5, 0.8, 1, 1.2, 1.5, 2, 2.5, 3, 3.5, 4, 4.5, 5 MPa and other values therein). In other embodiments, pulse averages across the volumetric field (average of all planes) could be 0.05-2 MPa (e.g., 0.05, 0.08, 0.10, 0.15, 0.20, 0.25, 0.30, 0.35, 0.40, 0.45, 0.50, 0.55, 0.6, 0.7, 0.8, 0.9, 1.0, 1.1, 1.2, 1.3, 1.4, 1.5, 1.6, 1.7, 1.8, 1.9, 2.0 and other values therein).

FIG. 40B is a graphical representation of average and peak pressures by plane according to one embodiment in which a prescribed set of phase combinations increases attenuation of the peak average pressures across the field compared to the embodiment of FIG. 40A. The embodiment illustrated with FIG. 40B of a prescribed set of phase combinations that increases attenuation of the peak average pressures across the field thereby substantially flattens the average pressure profile across the field, resulting in a more uniform pressure field. In some embodiments, the prescribed set of phase combinations create an incoherent field. In FIG. 40A a peak in the average pressure occurs at z=102 mm, and this peak has a pressure that is several times higher than most of the remainder of the field. By applying the approach described with a non-invasive sonodynamic therapy system with a filtering step to prescribe unique phasing for each element in an array for creating a large volumetric field, such as illustrated in FIG. 8B, alternative sets of waveform phase combinations for each of the transducer elements are filtered and selected to attenuate the peak pressure locations in the field, resulting in the ability to drive a substantially more uniform average pressure and more incoherence across the field, as shown in FIG. 40B. In one embodiment, filtering and prescribing a set of phases produces a broader and more uniform ultrasound field by avoiding combinations that create unwanted peaks in the field, and thereby enable the transducer to be driven to produce a larger and more homogenous therapeutic field. FIG. 8B illustrates an embodiment with this approach of filtering and prescribing a set of phase combinations that substantially flattens the average and peak pressure profiles across the volumetric pressure field, providing a substantially more uniform volumetric pressure field, which can be driven to produce a larger overall volumetric field. In some embodiments, the prescribed set of phase combinations create an incoherent field.

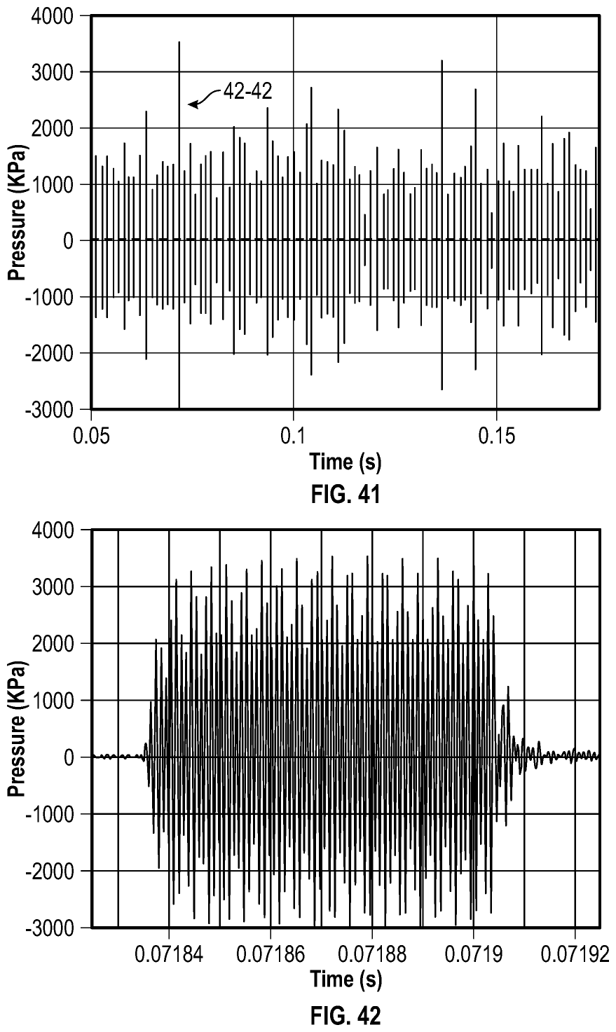

FIG. 41 illustrations a portion of a hydrophone recoding at a fixed point in the volumetric field, showing the variance of the pressure seen at each location over time (˜0.05-0.175 seconds). With each sequential burst providing a unique driving pattern, the magnitude of the pressure varies on a burst by burst basis. The phase patterns are random, and the wavefronts from multiple elements do not form a tight focus. The effect of this can be seen in the time-series waveform in FIG. 41, which is a zoomed in view over time (˜0.0718-0.0719 seconds) of burst 42-42 in FIG. 41, looking at a single 65 usec burst. FIG. 42 demonstrates that peak pressure events occur for only a small fraction of the 65 usec burst.

Conclusions from the Experiment: The peak pressure in the volumetric free field was determined to be 4.40 MPa for max therapy conditions. The maximum pulse average pressure for a single plane was determined to be 1.78 MPa for max therapy conditions. The maximum pulse average pressure across the volumetric field was determined to be 0.73 MPa for max therapy conditions. In other embodiments, volumetric peak instantaneous pressure could be in a range of 1-8 MPa (e.g., 1, 1.5, 2, 2.5, 3, 3.5, 4, 4.5, 5, 5.5, 6, 6.5, 7, 7.5, and/or 8 MPa and other values therein). In other embodiments, maximum pulse average pressure for a single plane could be 0.1-5 MPa (e.g., 0.1, 0.5, 0.8, 1, 1.2, 1.5, 2, 2.5, 3, 3.5, 4, 4.5, 5 MPa and other values therein). In other embodiments, pulse averages across the volumetric field (average of all planes) could be 0.05-2 MPa (e.g., 0.05, 0.08, 0.10, 0.15, 0.20, 0.25, 0.30, 0.35, 0.40, 0.45, 0.50, 0.55, 0.6, 0.7, 0.8, 0.9, 1.0, 1.1, 1.2, 1.3, 1.4, 1.5, 1.6, 1.7, 1.8, 1.9, 2.0 and other values therein). Various examples have been described with reference to certain disclosed embodiments. The various embodiments are presented for purposes of illustration and not limitation. Various changes, adaptations, and modifications can be made without departing from the scope of the disclosure or the scope of the appended claims.

The detailed description provides some practical illustrations and examples. Many of the noted examples, embodiments, and/or embodiments have a variety of suitable alternatives. A number of various examples of sonodynamic therapy devices are disclosed herein using the description provided in addition to the accompanying drawings according to various embodiments. Each of the embodiments disclosed herein can be employed independently or in combination with one or more (e.g., single, two, three, four, five, and any number including all) of the other embodiments disclosed herein. In various embodiments, an embodiment may comprise, consist essentially of, or consist of recited elements. While the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various examples described and the appended claims. Further, the disclosure herein of any particular feature, aspect, method, property, characteristic, quality, attribute, element, or the like in connection with an example can be used in all other examples set forth herein. Depending on the example, one or more acts, events, or functions of any of the algorithms, methods, or processes described herein can be performed in a different sequence, can be added, merged, or left out altogether (e.g., not all described acts or events are necessary for the practice of the algorithm). In some examples, acts or events can be performed concurrently, rather than sequentially. Further, no element, feature, block, or step, or group of elements, features, blocks, or steps, are necessary or indispensable to each example. Additionally, all possible combinations, sub-combinations, and rearrangements of systems, methods, features, elements, modules, blocks, and so forth are within the scope of this disclosure.

Unless otherwise defined, all technical and scientific terms used herein have the same meaning as commonly understood by one of ordinary skill in the art to which this disclosure pertains. As used herein, the term “about” includes values within 10%, 5%, or 1% of the recited values. For example, as used herein, the term “substantial” or “substantially” includes values within 10%, 5%, or 1% of the recited values. All publications, patent applications, patents, and other references mentioned herein are incorporated by reference in their entirety. In addition, the embodiments, aspects, materials, methods, and examples are illustrative only and not intended to be limiting. The term “aspect” includes non-limiting embodiments. When numbers are provided, values and ranges therein should be included where stated or understood from a fair reading. For example, disclosure of “10-50% (e.g., 10, 20, 30, 40, 50% and values and ranges therein)” would include 10-20%, 20-40%, etc. as well as 10%, 15%, 35% etc.). Any methods disclosed herein need not be performed in the order recited. The use of sequential, or time-ordered language, such as “then,” “next,” “after,” “subsequently,” and the like, unless specifically stated otherwise, or otherwise understood within the context as used, is generally intended to facilitate the flow of the text and is not intended to limit the sequence of operations performed. Thus, some examples may be performed using the sequence of operations described herein, while other examples may be performed following a different sequence of operations. The methods disclosed herein include certain actions taken by a practitioner; however, they can also include any third-party instruction of those actions, either expressly or by implication. For example, actions such as “oral administration of a drug” include “instructing the oral administration of a drug.”

Conditional language used herein, such as, among others, “can,” “might,” “may,” “e.g.,” and the like, unless specifically stated otherwise, or otherwise understood within the context as used, is generally intended to convey that some examples include, while other examples do not include, certain features, elements, and/or states. Thus, such conditional language is not generally intended to imply that features, elements, blocks, and/or states are in any way required for one or more examples or that one or more examples necessarily include logic for deciding, with or without author input or prompting, whether these features, elements and/or states are included or are to be performed in any particular example. The phrase “at least one of” is intended to require at least one item from the subsequent listing, not one type of each item from each item in the subsequent listing. For example, “at least one of A, B, and C” can include A; B; C; A and B; A and C; B and C; or A, B, and C.

The details of one or more aspects of various embodiments are set forth in the accompanying drawings and the description herein. Other features and advantages of the present disclosure will be apparent from the description and drawings, and from the claims appended hereto.

1. An ultrasound transducer system configured to produce a normalized acoustic pressure profile for activating a sonosensitizer in conjunction with providing sonodynamic therapy, the ultrasound transducer system comprising:
an alignment device; and
at least one ultrasound array, the at least one ultrasound array comprising a plurality of ultrasonic piezoelectric transducer elements configured to generate a normalized acoustic pressure profile for activating a sonosensitizer located within a tissue of a diseased organ of a patient,
wherein the normalized acoustic pressure profile comprises:
a peak pressure in a volumetric free field; and
an average pressure across the volumetric free field;
wherein the normalized acoustic pressure profile is configured to reduce a difference between the peak pressure and the average pressure in the volumetric free field,
wherein the normalized acoustic pressure profile is configured to produce the peak pressure in the volumetric free field that is in a range of 101%-400% of the average pressure in the volumetric free field;
wherein the plurality of piezoelectric ultrasonic transducer elements is driven by one or more of:
a modulated phase across the plurality of ultrasonic piezoelectric transducer elements, and
a modulated frequency across the plurality of ultrasonic piezoelectric transducer elements,
wherein each ultrasonic piezoelectric transducer element in the at least one ultrasound array of ultrasonic piezoelectric transducer elements comprises a planar emitting surface configured to emit an acoustic wave, wherein the acoustic wave is a defocused acoustic wave or an unfocused acoustic wave,
wherein the alignment device is configured to align the tissue of the diseased organ of the patient with the at least one ultrasound array.

2. The ultrasound transducer system of claim 1, wherein an unfocused acoustic wave comprises a planar acoustic wave.

3. The ultrasound transducer system of claim 1, wherein the normalized acoustic pressure profile further comprises:
a maximum pulse average pressure across the volumetric free field; and
a maximum pulse average pressure for a single plane in the volumetric free field;

4. The ultrasound transducer system of claim 1, wherein the normalized acoustic pressure profile is configured to provide for a consistent average pressure in the volumetric free field, wherein the normalized acoustic pressure profile is configured to produce an average pressure across an incoherent volumetric free field that is in a range of 10%-200%.

5. The ultrasound transducer system of claim 1, wherein a random phase drive pattern is configured to be filtered through a free field measurement to identify a phase drive pattern and a frequency drive pattern that attenuates peak pressure locations and results in a substantially uniform peak and average pressures throughout an incoherent pressure volume.

6. The ultrasound transducer system of claim 1, wherein a random phase drive pattern is configured to be filtered through numerical simulations to identify a phase drive pattern and a frequency drive pattern that results in a substantially uniform peak and average pressures throughout an incoherent pressure volume.

7. The ultrasound transducer system of claim 1, wherein a random phase drive pattern is configured to be filtered to remove patterns that produce an unintended coherence between one or more elements, thereby attenuating peak pressure locations to result in a more uniform field that can be driven to produce a larger therapeutic volume.

8. (canceled)

9. (canceled)

10. The ultrasound transducer system of claim 1, wherein the acoustic wave is amplitude modulated, frequency modulated, phase modulated, continuous, discontinuous, pulsed, randomized, or combinations thereof.

11. The ultrasound transducer system of claim 1, wherein a random phase drive pattern is configured to create an incoherent field.

12. The ultrasound transducer system of claim 1, wherein the normalized acoustic pressure profile is produced by at least one unique phase combination generated by a control algorithm to produce a uniform peak pressure and an average pressure across an incoherent ultrasound field in order to increase a treatment volume of the volumetric free field.

13. The ultrasound transducer system of claim 12, wherein the control algorithm is selected from a list of pre-screened phase sets, hydrophone measurements, simulations, or an analysis of disorder.

14. (canceled)

15. (canceled)

16. An ultrasound transducer system configured to produce a normalized acoustic pressure profile for activating a sonosensitizer in conjunction with providing sonodynamic therapy, the ultrasound transducer system comprising:
a plurality of ultrasound arrays, each ultrasound array of the plurality of ultrasound arrays comprising a housing comprising a plurality of ultrasonic transducer elements configured to generate a normalized acoustic pressure profile for activating a sonosensitizer located within a target tissue of patient,
wherein the normalized acoustic pressure profile comprises:
a peak pressure in a volumetric free field; and
an average pressure across the volumetric free field;
wherein the normalized acoustic pressure profile is configured to provide for a consistent average pressure across the volumetric free field,
wherein the normalized acoustic pressure profile is configured to produce an average pressure across the volumetric free field that is in a range of 1%-200% across each ultrasound array;
wherein the plurality of ultrasonic transducer elements is driven by one or more of:
a modulated phase across the plurality of ultrasonic transducer elements, and
a modulated frequency across the plurality of ultrasonic transducer elements,
wherein each ultrasonic transducer element in the ultrasound array comprises a planar emitting surface configured to emit an acoustic wave, wherein the acoustic wave is a defocused acoustic wave or an unfocused acoustic wave.

17. (canceled)

18. The ultrasound transducer system of claim 16, wherein a random phase drive pattern is configured to be filtered through a free field measurement to identify a phase drive pattern and a frequency drive pattern that attenuates peak pressure locations and results in a substantially uniform peak and average pressures throughout an incoherent pressure volume.

19. The ultrasound transducer system of claim 16, wherein a random phase drive pattern is configured to be filtered through numerical simulations to identify a phase drive pattern and a frequency drive pattern that results in a substantially uniform peak and average pressures throughout an incoherent pressure volume.

20. The ultrasound transducer system of claim 16, wherein a random phase drive pattern is configured to be filtered to remove patterns that produce an unintended coherence between one or more elements, thereby attenuating peak pressure locations to result in a more uniform field that can be driven to produce a larger therapeutic volume.

21. (canceled)

22. (canceled)

23. The ultrasound transducer system of claim 16, wherein a control algorithm is configured to produce a unique phase drive pattern for each element in the ultrasound array.

24. The ultrasound transducer system of claim 16, wherein the acoustic wave is amplitude modulated, frequency modulated, phase modulated, continuous, discontinuous, pulsed, randomized, or combinations thereof.

25. The ultrasound transducer system of claim 16, wherein a random phase drive pattern is configured to create an incoherent field.

26. The ultrasound transducer system of claim 16, wherein the normalized acoustic pressure profile is configured to provide for a consistent average pressure in the volumetric free field, wherein the normalized acoustic pressure profile is configured to produce an average pressure across an incoherent volumetric free field that is in a range of 10%-200%.

27. The ultrasound transducer system of claim 16, wherein the normalized acoustic pressure profile is produced by at least one unique phase combination generated by a control algorithm to produce a uniform peak pressure and an average pressure across an incoherent ultrasound field in order to increase a treatment volume of the volumetric free field.

28.-83. (canceled)
